(19) United States
(12) Patent Application Publication
(10) Pub. No.: US 2021/0025890 A1
(43) Pub. Date: Jan. 28, 2021

(54) METHODS FOR DETECTING AND QUANTIFYING FGF21

(71) Applicant: GENENTECH, INC., South San Francisco, CA (US)

(72) Inventors: John Hok Nin Lowe, Dublin, CA (US); Junichiro Sonoda, Lafayette, CA (US)

(73) Assignee: GENENTECH, INC., South San Francisco, CA (US)

(21) Appl. No.: 17/063,640

(22) Filed: Oct. 5, 2020

Related U.S. Application Data
(63) Continuation of application No. PCT/US2019/025726, filed on Apr. 4, 2019.
(60) Provisional application No. 62/652,701, filed on Apr. 4, 2018.

(51) Int. Cl. G01N 33/577; G01N 33/557; G01N 33/74

(52) U.S. Cl. CPC G01N 33/577; G01N 2333/50; G01N 33/74; G01N 33/557

(57) ABSTRACT

The presently disclosed subject matter provides antibodies that bind FGF21 and methods of using the same. In particular, the present disclosure provides immunoassay methods for detecting and quantifying active and total FGF21 levels in a sample and kits for performing such methods.

### CROSS REFERENCE TO RELATED APPLICATIONS

This application is a continuation of International Patent Application No. PCT/US2019/025726, filed Apr. 4, 2019, which claims the benefit of U.S. Provisional Application No. 62/652,701, filed Apr. 4, 2018, the disclosures of both of which are incorporated herein by reference in their entireties.

### SEQUENCE LISTING

The present application contains a Sequence Listing which has been submitted in ASCII format via EFS-Web and is hereby incorporated by reference in its entirety. Said ASCII copy, created on Apr. 2, 2019, is named 00B206_0968_SL.txt and is 105,640 bytes in size.

### FIELD OF THE INVENTION

The present invention relates to antibodies that bind to FGF21 as well as immunoassay methods and kits using the same.

### BACKGROUND

Fibroblast growth factor 21 (FGF21) is an endocrine member of the FGF superfamily and plays a role in the regulation of glucose and lipid metabolism. FGF21 requires FGF-receptor (FGFR) isoforms and the membrane-bound co-receptor Klotho-beta (KLB) for signaling (Ogawa et al. Proc. Natl. Acad. Sci. USA 104(18):7432-37 (2007); US 2010/0184665). FGF21 is a potent disease-modifying protein that has beneficial effects on glucose homeostasis and insulin sensitivity, and has been shown to reverse obesity and type 2 diabetes in animal disease models (Kharitonenkov et al. J Clin. Invest. 115(6): 1627-35 (2005)). The administration of recombinant FGF21 has been shown to reduce hepatic lipids, improve insulin sensitivity, and normalize glycemic control in leptin-signaling-deficient (ob/ob or db/db) mice or high-fat diet (HFD)-fed mice (Dunshee et al. J Biol. Chem. 291(11):5986-96 (2016); US 2015/0218276). Reduction in blood glucose and improvements in various cardiovascular risk factors have also been observed in obese and diabetic rhesus monkeys treated daily with recombinant FGF21.

FGF21 can be proteolytically cleaved at both the N-terminus and the C-terminus, and such cleavage has been shown to affect the activity of FGF21. At the N-terminus, the first four amino acids, which have the sequence His-Pro-Ile-Pro (HPIP (SEQ ID NO: 76)) in human FGF21 can be cleaved by a dipeptidyl peptidase (Dunshee et al. (2016)). At the C-terminus, the endopeptidase fibroblast activation protein (FAP) cleaves the most terminal 10 amino acids, which have the amino acid sequence Ser-Gln-Gly-Arg-Ser-Pro-Ser-Tyr-Ala-Ser (SQGRSPSYAS (SEQ ID NO: 77)) in human FGF21 (Dunshee et al. (2016)). FGF21 that lacks the four N-terminal amino acids is fully active; whereas, FGF21 that lacks the last ten C-terminal amino acids cannot bind the co-receptor KLB and is inactive (Yie et al. FEBS Letters 583:19-24 (2009)).

Circulating FGF21 has been proposed to be a biomarker for metabolic disorders such as diabetes as increased serum levels of FGF21 were observed in obese subjects, in subjects with nonalcoholic fatty liver disease (NAFLD) and in subjects with type 2 diabetes (Zhang et al. Diabetes 57(5):1246-1253 (2008); Li et al. Diabetes Res. Clin. Pract. 93(1):10⁻¹⁶ (2011)). Given the significant role for FGF21 in the treatment and development of metabolic disorders, there remains a need in the art for assays for determining the amount of FGF21 protein in an individual.

### SUMMARY

The present disclosure provides antibodies that bind Fibroblast growth factor 21 (FGF21) and use of such antibodies in immunoassay methods for the detection and quantification of FGF21 protein, e.g., total and/or active FGF21 protein, in a sample.

In certain embodiments, the present disclosure provides immunoassays for determining the amount of total FGF21 protein in a sample. For example, but not by way of limitation, the method to determine the amount of total FGF21 protein in a sample can include contacting a capture antibody that binds to an epitope present within amino acid residues 5-172 of FGF21 with the sample to generate a sample-capture antibody combination material, (b) contacting the sample-capture antibody combination material with a detector antibody that binds to an epitope present within amino acid residues 5-172 of FGF21, (c) detecting the detector antibody bound to the sample-capture antibody combination material and (d) calculating an amount of total FGF21 protein present in the sample based on the level of the detector antibody bound. In certain embodiments, the capture antibody and the detector antibody bind to different epitopes within amino acid residues 5-172 of FGF21.

In certain embodiments, the present disclosure provides immunoassays for determining the amount of active FGF21 protein in a sample. For example, but not by way of limitation, the method to determine the amount of active FGF21 protein in a sample can include (a) contacting a capture antibody that binds to an epitope present within amino acid residues 5-172 of FGF21 with the sample to generate a sample-capture antibody combination material, (b) contacting the sample-capture antibody combination material with a detector antibody that binds to an epitope present within amino acid residues 173-182 of FGF21, (c) detecting the detector antibody bound to the sample-capture antibody combination material and (d) calculating an amount of active FGF21 protein present in the sample based on the level of the detector antibody bound.

In certain embodiments, the present disclosure provides immunoassays for determining the ratio of active FGF21 protein to total FGF21 protein in a sample. For example, but not by way of limitation, the method can include (i) contacting a first capture antibody that binds to an epitope present within amino acid residues 5-172 of FGF21 with the sample to generate a first sample-capture antibody combination material, (ii) contacting the first sample-capture antibody combination material with a first detector antibody that binds to an epitope present within amino acid residues 5-172 of FGF21, (iii) detecting the first detector antibody bound to the sample-capture antibody combination material and (iv) calculating an amount of total FGF21 protein present in the sample based on the level of the first detector antibody bound. In certain embodiments, the method can further include (i) contacting a second capture antibody that binds to an epitope present within amino acid residues 5-172 of FGF21 with the sample to generate a second sample-capture antibody combination material, (ii) contacting the second sample-capture antibody combination material with a second detector antibody that binds to an epitope present within amino acid residues 173-182 of FGF21, (iii) detecting the second detector antibody bound to the sample-capture antibody combination material and (iv) calculating an amount of active FGF21 protein present in the sample based on the level of the second detector antibody bound. In certain embodiments, the method can include comparing the calculated amount of total FGF21 protein with the calculated amount of active FGF21 protein to determine the ratio of active FGF21 protein to total FGF21 protein in the sample. In certain embodiments, the first capture antibody and second capture antibody are the same antibody. In certain embodiments, the first capture antibody and the first detector antibody bind to different epitopes within amino acid residues 5-172 of FGF21.

In certain embodiments, the immunoassay method is an enzyme-linked immunosorbent assay (ELISA). In certain embodiments, the immunoassay method detects the amount of total or active FGF21 protein in the sample at an in-well sensitivity from about 2 pg/ml to about 20 pg/ml.

In certain embodiments, the immunoassay method is a single molecule detection assay, e.g., that uses the Quanterix Simoa HD-1 Analyzer™. In certain embodiments, the immunoassay method detects the amount of total or active FGF21 protein in the sample at an in-well sensitivity from about 0.2 pg/ml to about 0.5 pg/ml.

The present disclosure further provides kits for performing immunoassay methods for the detection and quantification of FGF21 protein. In certain embodiments, the present disclosure provides kits for determining the amount of total FGF21 protein in a sample. For example, but not by way of limitation, the kit for quantifying the amount of total FGF21 protein includes (a) a capture antibody that binds to an epitope present within amino acid residues 5-172 of FGF21, (b) a detector antibody that binds to an epitope present within amino acid residues 5-172 of FGF21 and (c) a detection agent. In certain embodiments, the capture antibody and the detector antibody bind to different epitopes within amino acid residues 5-172 of FGF21.

In certain embodiments, the present disclosure provides kits for determining the amount of active FGF21 protein in a sample. For example, but not by way of limitation, the kit for quantifying the amount of active FGF21 protein includes (a) a capture antibody that binds to an epitope present within amino acid residues 5-172 of FGF21, (b) a detector antibody that binds to an epitope present within amino acid residues 173-182 of FGF21 and (c) a detection agent.

In certain embodiments, the present disclosure provides kits for determining the amount of active FGF21 protein in a sample. For example, but not by way of limitation, the kit for determining the ratio of active FGF21 protein to total FGF21 protein in a sample can include (a) a first capture antibody that binds to an epitope present within amino acid residues 5-172 of FGF21, (b) a first detector antibody that binds to an epitope present within amino acid residues 5-172 of FGF21, (c) a second capture antibody that binds to an epitope present within amino acid residues 5-172 of FGF21, (d) a second detector antibody that binds to an epitope present within amino acid residues 173-182 of FGF21 and (e) one or more detection agents. In certain embodiments, the first capture antibody and second capture antibody are the same antibody. In certain embodiments, the first capture antibody and the first detector antibody bind to different epitopes within amino acid residues 5-172 of FGF21.

In certain embodiments, the detection agent for detecting the detector antibody, first detector antibody and/or second detector antibody can be selected from a group consisting of a streptavidin-β-D-galactopyranose conjugate, a streptavidin-horseradish peroxidase conjugate and a combination thereof. In certain embodiments, the streptavidin-β-D-galactopyranose conjugate has a concentration from about 100 pM to about 400 pM.

In certain embodiments, a kit of the present disclosure can further include resorufin β-D-galactopyranoside, tetramethylbenzidine, hydrogen peroxide or combinations thereof. For example, but not by way of limitation, a kit of the present disclosure can include a streptavidin-β-D-galactopyranose conjugate as the detection agent and can further include resorufin β-D-galactopyranoside. In certain embodiments, a kit of the present disclosure can include a streptavidin-horseradish peroxidase conjugate as the detection agent and can further include tetramethylbenzidine and hydrogen peroxide.

In certain embodiments, a kit disclosed herein detects the amount of total or active FGF21 protein in the sample at an in-well sensitivity from about 2 pg/ml to about 20 pg/ml. In certain embodiments, a kit disclosed herein detects the amount of total or active FGF21 protein in the sample at an in-well sensitivity from about 0.2 pg/ml to about 0.5 pg/ml.

In certain embodiments, the capture antibody, first capture antibody or second capture antibody is immobilized to a paramagnetic bead. In certain embodiments, the capture antibody, first capture antibody and/or second capture antibody binds to FGF21 with a K_{d} from about 10⁻¹⁰ M to 10⁻¹³ M. In certain embodiments, the detector antibody, first detector antibody and second detector antibody is conjugated to biotin. In certain embodiments, the detector antibody and/or first detector antibody binds to FGF21 with a K_{d} from about 10⁻¹⁰ M to 10⁻¹³ M. In certain embodiments, the detector antibody and/or first detector antibody for use in determining the amount of total FGF21 protein has a concentration from about 0.1 μg/ml to about 1 μg/ml. In certain embodiments, the detector antibody and/or second detector antibody for use in determining the amount of active FGF21 protein has a concentration from about 1 μg/ml to about 3 μg/ml.

In certain embodiments, the capture antibody, first capture antibody and/or second capture antibody includes or competitively binds to an antibody that includes: (a) a heavy chain variable region CDR1 comprising an amino acid sequence selected from the group consisting of SEQ ID NOs: 26 and 27, e.g., 26, and conservative substitutions thereof, (b) a heavy chain variable region CDR2 domain comprising an amino acid sequence selected from the group consisting of SEQ ID NOs: 30 and 31, e.g., 30, and conservative substitutions thereof, (c) a heavy chain variable region CDR3 domain comprising an amino acid sequence selected from the group consisting of SEQ ID NOs: 34 and 35, e.g., 34, and conservative substitutions thereof, (d) a light chain variable region CDR1 domain comprising an amino acid sequence selected from the group consisting of SEQ ID NOs: 38 and 39, e.g., 38, and conservative substitutions thereof, (e) a light chain variable region CDR2 domain comprising an amino acid sequence selected from the group consisting of SEQ ID NOs: 42 and 43, e.g., 42, and conservative substitutions thereof and (f) a light chain variable region CDR3 domain comprising an amino acid sequence selected from the group consisting of SEQ ID NOs: 46 and 47, e.g., 46, and conservative substitutions thereof.

In certain embodiments, the capture antibody, first capture antibody and/or second capture antibody includes or competitively binds to an antibody that includes: (a) a heavy chain variable region comprising an amino acid sequence selected from the group consisting of SEQ ID NOs: 54, 55, 74 and 75, e.g., 54, and conservative substitutions thereof; and (b) a light chain variable region comprising an amino acid sequence selected from the group consisting of SEQ ID NOs: 50, 51, 70 and 71, e.g., 50, and conservative substitutions thereof. In certain embodiments, the capture antibody, first capture antibody and/or second capture antibody includes or competitively binds to an antibody that includes: (a) a heavy chain comprising an amino acid sequence selected from the group consisting of SEQ ID NOs: 22, 23, 66 and 67, e.g., 22, and conservative substitutions thereof; and (b) a light chain comprising an amino acid sequence selected from the group consisting of SEQ ID NOs: 18, 19, 62 and 63, e.g., 18, and conservative substitutions thereof.

In certain embodiments, the detector antibody and/or first detector antibody includes or competitively binds to an antibody that includes: (a) a heavy chain variable region CDR1 comprising an amino acid sequence selected from the group consisting of SEQ ID NOs: 28 and 29, e.g., 29, and conservative substitutions thereof, (b) a heavy chain variable region CDR2 domain comprising an amino acid sequence selected from the group consisting of SEQ ID NOs: 32 and 33, e.g., 33, and conservative substitutions thereof, (c) a heavy chain variable region CDR3 domain comprising an amino acid sequence selected from the group consisting of SEQ ID NOs: 36 and 37, e.g., 37, and conservative substitutions thereof, (d) a light chain variable region CDR1 domain comprising an amino acid sequence selected from the group consisting of SEQ ID NOs: 40 and 41, e.g., 41, and conservative substitutions thereof, (e) a light chain variable region CDR2 domain comprising an amino acid sequence selected from the group consisting of SEQ ID NOs: 44 and 45, e.g., 45, and conservative substitutions thereof and (f) a light chain variable region CDR3 domain comprising an amino acid sequence selected from the group consisting of SEQ ID NOs: 48 and 49, e.g., 49, and conservative substitutions thereof.

In certain embodiments, the detector antibody and/or first detector antibody includes or competitively binds to an antibody that includes: (a) a heavy chain variable region comprising an amino acid sequence selected from the group consisting of SEQ ID NOs: 56, 57, 72 and 73, e.g., 57, and conservative substitutions thereof; and (b) light chain variable region comprising an amino acid sequence selected from the group consisting of SEQ ID NOs: 52, 53, 68 and 69, e.g., 53, and conservative substitutions thereof. In certain embodiments, the detector antibody and/or first detector antibody includes or competitively binds to an antibody that includes: (a) a heavy chain comprising an amino acid sequence selected from the group consisting of SEQ ID NOs: 24, 25, 64 and 65, e.g., 25, and conservative substitutions thereof; and (b) a light chain comprising an amino acid sequence selected from the group consisting of SEQ ID NOs: 20, 21, 60 and 61, e.g., 21, and conservative substitutions thereof.

In certain embodiments, an antibody used in the disclosed immunoassay methods can be a monoclonal antibody, a chimeric antibody, a humanized antibody or a human antibody. In certain embodiments, an antibody used in the disclosed immunoassay methods can be an antibody fragment, e.g., a Fv, Fab, Fab′, scFv, diabody or F(ab′)2 fragment.

In certain embodiments, the sample being analyzed is a blood sample obtained from a subject. In certain embodiments, the sample is a plasma sample obtained from a subject.

The present disclosure further provides isolated anti-FGF21 antibodies. In certain embodiments, an isolated anti-FGF21 antibody, or an antigen-binding portion thereof, comprises: (a) a heavy chain variable region CDR1 comprising an amino acid sequence selected from the group consisting of SEQ ID NOs: 26-29, and conservative substitutions thereof; (b) a heavy chain variable region CDR2 domain comprising an amino acid sequence selected from the group consisting of SEQ ID NOs: 30-33, and conservative substitutions thereof; (c) a heavy chain variable region CDR3 domain comprising an amino acid sequence selected from the group consisting of SEQ ID NOs: 34-37, and conservative substitutions thereof; (d) a light chain variable region CDR1 domain comprising an amino acid sequence selected from the group consisting of SEQ ID NOs: 38-41, and conservative substitutions thereof; (e) a light chain variable region CDR2 domain comprising an amino acid sequence selected from the group consisting of SEQ ID NOs: 42-45, and conservative substitutions thereof; and (f) a light chain variable region CDR3 domain comprising an amino acid sequence selected from the group consisting of SEQ ID NOs: 46-49, and conservative substitutions thereof.

In certain embodiments, an isolated anti-FGF21 antibody, or an antigen-binding portion thereof, comprises: (a) a heavy chain variable domain (VH) sequence comprising an amino acid sequence selected from the group consisting of SEQ ID NOs: 54-57 and 72-75; and (b) a light chain variable domain (VH) sequence comprising an amino acid sequence selected from the group consisting of SEQ ID NOs: 50-53 and 68-71. In certain embodiments, an isolated anti-FGF21 antibody, or an antigen-binding portion thereof, comprises: (a) a heavy chain sequence comprising an amino acid sequence selected from the group consisting of SEQ ID NOs: 22-25 and 64-67; and (b) a light chain sequence comprising an amino acid sequence selected from the group consisting of SEQ ID NOs: 18-21 and 60-63.

### BRIEF DESCRIPTION OF THE FIGURES

FIG. 1. Depicts the results of an ELISA screen of 80 hybridoma supernatants expressing anti-FGF21 antibodies.

FIG. 2: Depicts the dose response of intact versus cleaved FGF21 detection by sandwich ELISA using mAb4 or mAb9 capture antibodies and the mAb11 detector antibody.

FIG. 3: Depicts the BIACORE® surface plasmon resonance analysis of anti-FGF21 antibodies mAb4, mAb9, mAb11 and mAb15.

FIG. 4: Depicts a schematic diagram showing anti-FGF21 antibody binding to FGF21 (FGF19 is used as negative control).

FIG. 5: Depicts a schematic diagram of a non-limiting embodiment of colorimetric ELISA methods for detecting total FGF21 and active FGF21.

FIG. 6: Depicts a non-limiting embodiment of a protocol for performing total and active FGF21 ELISA assays.

FIG. 7: Depicts the results of ELISA assays using either mAb4 or mAb1 capture antibodies and various detection antibodies.

FIG. 8: Depicts a comparison of the sensitivity of detecting wild-type and cleaved human FGF21 using exemplary total and active FGF21 ELISA assays.

FIG. 9: Depicts detection of human FGF21 using an exemplary total FGF21 ELISA assay.

FIG. 10: Depicts an ELISA assay indicating that exemplary anti-FGF21 antibodies do not cross-react with mouse FGF21.

FIG. 11: Depicts a comparison of the sensitivities of capture antibodies mAb4 and mAb9 in exemplary total and active FGF21 ELISA assays.

FIG. 12: Depicts the effect of coat buffer and concentration on the sensitivity of an exemplary total FGF21 ELISA assay using mAb4 as the capture antibody and mAb15 as the detector antibody.

FIG. 13: Depicts the effect of coat buffer and concentration on the sensitivity of an exemplary active FGF21 ELISA assay using mAb4 as the capture antibody and the sheep C-terminal pAb as the detector antibody.

FIG. 14: Depicts the effect of biotin-conjugated detector antibody and HRP concentration on the sensitivity of an exemplary total FGF21 ELISA assay using mAb4 as the capture antibody and mAb15 as the detector antibody.

FIG. 15: Depicts a schematic diagram of a non-limiting embodiment of single molecule detection methods for detecting total FGF21 and active FGF21 using the Quanterix Simoa HD-1 Analyzer™ (“Quanterix Simoa”).

FIG. 16: Depicts a non-limiting embodiment of a two-step assay protocol for exemplary total FGF21 and active FGF21 assays using the Quanterix Simoa.

FIG. 17: Depicts the dose response of intact versus cleaved FGF21 detection by exemplary total FGF21 and active FGF21 assays using the Quanterix Simoa.

FIG. 18: Depicts a non-limiting embodiment of a protocol for performing exemplary total and active FGF21 assays using the Quanterix Simoa.

FIG. 19: Depicts the standard curves in exemplary total and active FGF21 assays using the Quanterix Simoa.

FIG. 20: Depicts the standard curve performance in exemplary total and active FGF21 assays using the Quanterix Simoa.

FIG. 21: Depicts a comparison of the sensitivity of detecting total and active FGF21 in the presence of BA010 and IL-12 buffers in exemplary total and active FGF21 assays using the Quanterix Simoa.

FIG. 22: Depicts the effect of high bead (HB) and low bead (LB) concentrations on the sensitivity of exemplary total and active FGF21 assays using the Quanterix Simoa.

FIG. 23: Depicts a comparison of the sensitivity of detecting total and active FGF21 using three capture paramagnetic bead lots in exemplary total and active FGF21 assays using the Quanterix Simoa.

FIG. 24: Depicts a comparison of the sensitivity of detecting total and active FGF21 using various detection antibodies in an exemplary total FGF21 assay using the Quanterix Simoa.

FIG. 25: Depicts an analysis of the hook effect in an exemplary total FGF21 assay using mAb4 as the capture antibody and mAb15 as the detector antibody using the Quanterix Simoa.

FIG. 26: Depicts the detection of total FGF21 and active FGF21 in plasma and serum samples from a healthy donor using exemplary total and active FGF21 ELISA assays.

FIG. 27: Depicts the detection of total FGF21 and active FGF21 in plasma samples or plasma samples treated with MS-SAFE from donors that are hypertensive and donors that are not on medication using exemplary total and active FGF21 ELISA assays.

FIG. 28A: Depicts the detection of total FGF21 and active FGF21 in plasma samples from healthy and type 2 diabetic patients using exemplary total and active FGF21 assays (Day 1) using the Quanterix Simoa.

FIG. 28B: Depicts the detection of total FGF21 and active FGF21 in plasma samples from healthy and type 2 diabetic patients using exemplary total and active FGF21 assays (Day 2) using the Quanterix Simoa.

FIG. 29: Depicts the reproducibility of exemplary total and active FGF21 assays used for the detection of total FGF21 and active FGF21 in plasma samples from healthy and type 2 diabetic patients using the Quanterix Simoa.

FIG. 30: Depicts the linearity of dilution of exemplary total and active FGF21 assays used for the detection of total FGF21 and active FGF21 in plasma samples from type 2 diabetic patients using the Quanterix Simoa.

FIG. 31: Depicts the determination of lower limit of quantification (LLOQ) in exemplary total and active FGF21 assays used for the detection of total FGF21 and active FGF21 in plasma samples from type 2 diabetic patients using the Quanterix Simoa.

FIG. 32: Depicts the specificity of exemplary total and active FGF21 assays used for the detection of total FGF21 and active FGF21 in plasma samples from type 2 diabetic patients using the Quanterix Simoa.

FIG. 33: Depicts the detection of total FGF21 and active FGF21 in plasma samples prepared using P800 or K₂-EDTA using exemplary total and active FGF21 assays using the Quanterix Simoa.

FIG. 34: Depicts an analysis of total FGF21 and active FGF21 detected in P800 and K₂-EDTA plasma samples from the GC29819 study in exemplary total and active FGF21 assays using the Quanterix Simoa.

FIG. 35: Depicts a correlation between the amount of total FGF21 and active FGF21 detected in P800 and K₂-EDTA plasma samples (GC29819 clinical Study) quantitated using an exemplary total FGF21 assay using the Quanterix Simoa.

FIG. 36: Depicts a correlation between the amount of total FGF21 and active FGF21 detected in P800 and K₂-EDTA plasma samples (GC29819 Study) quantitated using an exemplary active FGF21 assay using the Quanterix Simoa.

FIG. 37: Depicts an evaluation of the stability of the P800 plasma samples from the GC29819 study using exemplary total and active FGF21 assays using the Quanterix Simoa.

FIG. 38: Depicts the effect of assay diluent containing 10 μg/ml of mouse or sheep IgG on the total and active assays using the Quanterix Simoa.

FIG. 39: Depicts the effect of assay diluent containing 10 μg/ml of mouse and sheep IgG on the total and active assays using the Quanterix Simoa.

FIG. 40: Depicts the effect of assay diluent containing 10 μg/ml of mouse or sheep IgG on the standard curves on the total and active assays using the Quanterix Simoa.

FIG. 41A: Depicts the sequences of the light chain variable regions of exemplary anti-FGF21 antibodies. Light chain variable region sequences are disclosed as SEQ ID NOs: 50, 51, 52, 53, 71, 70, 69 and 68, respectively, in order of appearance. CDR-L1 sequences are disclosed as SEQ ID NOs: 38, 39, 40, 41, 38, 39, 40 and 41, respectively, in order of appearance; CDR-L2 sequences are disclosed as SEQ ID NOs: 42, 43, 44, 45, 42, 43, 44 and 45, respectively, in order of appearance; and CDR-L3 sequences are disclosed as SEQ ID NOs: 46, 47, 48, 49, 46, 47, 48 and 49, respectively, in order of appearance.

FIG. 41B: Depicts the sequences of the heavy chain variable regions of exemplary anti-FGF21 antibodies. Heavy chain variable region sequences are disclosed as SEQ ID NOs: 54, 55, 56, 57, 75, 74, 73 and 72, respectively, in order of appearance. CDR-H1 sequences are disclosed as SEQ ID NOs: 26, 27, 28, 29, 26, 27, 28 and 29, respectively, in order of appearance; CDR-H2 sequences are disclosed as SEQ ID NOs: 30, 31, 32, 33, 30, 31, 32 and 33, respectively, in order of appearance; and CDR-H3 sequences are disclosed as SEQ ID NOs: 34, 35, 36, 37, 34, 35, 36 and 37, respectively, in order of appearance.

### DETAILED DESCRIPTION

For clarity, but not by way of limitation, the detailed description of the presently disclosed subject matter is divided into the following subsections:

I. Definitions;

II. Immunoassays;

III. Antibodies;

IV. Kits; and

V. Exemplary Embodiments.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.”

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

The term “Fibroblast growth factor 21” or “FGF21,” as used herein, refers to any native FGF21 from any vertebrate source, including mammals such as primates (e.g., humans) and rodents (e.g., mice and rats), unless otherwise indicated. The term encompasses “full-length,” unprocessed FGF21 as well as any form of FGF21 that results from processing in the cell. The term also encompasses naturally occurring variants of FGF21, e.g., splice variants or allelic variants, unless otherwise indicated. A non-limiting example of a full-length human FGF21 amino acid is shown below:

(SEQ ID NO: 1)
HPIPDSSPLLQFGGQVRQRYLYTDDAQQTEAHLEIREDGTVGGAADQSPE
SLLQLKALKPGVIQILGVKTSRFLCQRPDGALYGSLHFDPEACSFRELLL
EDGYNVYQSEAHGLPLHLPGNKSPHRDPAPRGPARFLPLPGLPPALPEPP
GILAPQPPDVGSSDPLSMVGPSQGRSPSYAS.

The term “total FGF21,” as used herein, includes unprocessed forms of FGF21 as well as all forms of FGF21 that result from cellular processing, e.g., N-terminally-cleaved FGF21 and C-terminally-cleaved FGF21. A non-limiting example of a human FGF21 amino acid that lacks the ten C-terminal amino acids is:

(SEQ ID NO: 58)
HPIPDSSPLLQFGGQVRQRYLYTDDAQQTEAHLEIREDGTVGGAADQSPE
SLLQLKALKPGVIQILGVKTSRFLCQRPDGALYGSLHFDPEACSFRELLL
EDGYNVYQSEAHGLPLHLPGNKSPHRDPAPRGPARFLPLPGLPPALPEPP
GILAPQPPDVGSSDPLSMVGP.

A non-limiting example of a human FGF21 amino acid that lacks the 4 N-terminal amino acids is:

(SEQ ID NO: 59)
DSSPLLQFGGQVRQRYLYTDDAQQTEAHLEIREDGTVGGAADQSPESLLQ
LKALKPGVIQILGVKTSRFLCQRPDGALYGSLHFDPEACSFRELLLEDGY
NVYQSEAHGLPLHLPGNKSPHRDPAPRGPARFLPLPGLPPALPEPPGILA
PQPPDVGSSDPLSMVGPSQGRSPSYAS.

For example, but not by way of limitation, the term “total FGF21” includes FGF21 proteins that have the amino acid sequence set forth in SEQ ID NO: 1, SEQ ID NO: 58 or SEQ ID NO: 59.

The term “active FGF21,” as used herein, refers to an FGF21 protein that retains its C-terminal fragment. In certain embodiments, the term includes processed forms of FGF21, such as those where the N-terminal fragment of FGF21, e.g., amino acid residues 1-4 of SEQ ID NO: 1, has been cleaved. For example, but not by way of limitation, the term “active FGF21” includes FGF21 proteins that have the amino acid sequence set forth in SEQ ID NO: 1 or the amino acid sequence set forth in SEQ ID NO: 59.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv); and multispecific antibodies formed from antibody fragments.

An antibody “which binds” an antigen of interest, e.g., a FGF21 protein, is one that binds the antigen with sufficient affinity such that the antibody is useful as an assay reagent, e.g., as a capture antibody or as a detection antibody. Typically, such an antibody does not significantly cross-react with other polypeptides. With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a target molecule compared to binding of a control molecule, which generally is a molecule of similar structure that does not have binding activity.

The term “anti-FGF21 antibody” refers to an antibody that is capable of binding FGF21 with sufficient affinity such that the antibody is useful as an agent in targeting FGF21, e.g., as an agent in the assays described herein. In certain embodiments, the extent of binding of an anti-FGF21 antibody to an unrelated, non-FGF21 protein is less than about 10% of the binding of the antibody to FGF21 as measured, e.g., by a radioimmunoassay (RIA). In certain embodiments, an antibody that binds to FGF21 has a dissociation constant (K_{d}) of ≤1 M, ≤100 mM, ≤10 mM, ≤1 mM, ≤100 μM, ≤10 μM, ≤1 μM, ≤100 nM, ≤10 nM, ≤1 nM, ≤0.1 nM, ≤0.01 nM or ≤0.001 nM. In certain embodiments, the K_{d} of an antibody that binds to FGF21, disclosed herein, can be 10⁻³ M or less or 10⁻⁸ M or less, e.g., from 10⁻⁸ M to 10⁻¹³ M, e.g., from 10⁻⁹ M to 10⁻¹³ M. In certain embodiments, the K_{d} of an antibody that binds to FGF21, disclosed herein, can be 10⁻¹⁰ M to 10⁻¹³ M. In certain embodiments, an anti-FGF21 antibody binds to an epitope of FGF21 that is conserved among FGF21 from different species.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In certain embodiments, the number of amino acid changes are 10 or less, 9 or less, 8 or less, 7 or less, 6 or less, 5 or less, 4 or less, 3 or less, or 2 or less. In certain embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (CDRs), compared to a parent antibody which does not possess such alterations, such alterations resulting in an improvement in the affinity of the antibody for antigen.

An “antibody that competes for binding” with a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is described in “Antibodies,” Harlow and Lane (Cold Spring Harbor Press, Cold Spring Harbor, N.Y.).

A “capture antibody,” as used herein, refers to an antibody that specifically binds a target molecule, e.g., a form of FGF21, in a sample. Under certain conditions, the capture antibody forms a complex with the target molecule such that the antibody-target molecule complex can be separated from the rest of the sample. In certain embodiments, such separation may include washing away substances or material in the sample that did not bind the capture antibody. In certain embodiments, a capture antibody may be attached to a solid support surface, such as, for example but not limited to, a plate or a bead, e.g., a paramagnetic bead.

A “detection antibody,” as used herein, refers to an antibody that specifically binds a target molecule in a sample or in a sample-capture antibody combination material. Under certain conditions, the detection antibody forms a complex with the target molecule or with a target molecule-capture antibody complex. A detection antibody is capable of being detected either directly through a label, which may be amplified, or indirectly, e.g., through use of another antibody that is labeled and that binds the detection antibody. For direct labeling, the detection antibody is typically conjugated to a moiety that is detectable by some means, for example, including but not limited to, biotin or ruthenium.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1 q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (CDR) residues. The FR of a variable domain generally consists of four FR domains: FR1, FR2, FR3, and FR4. Accordingly, the CDR and FR sequences generally appear in the following sequence in VH (or VL): FR1-H1(L1)-FR2-H2(L2)-FR3-H3(L3)-FR4.

The terms “full-length antibody,” “intact antibody” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In certain embodiments, for the VL, the subgroup is subgroup kappa I as in Kabat et al., supra. In certain embodiments, for the VH, the subgroup is subgroup III as in Kabat et al., supra.

A “humanized” antibody refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “CDR,” as used herein, refers to each of the regions of an antibody variable domain which are hypervariable in sequence (also referred to herein as “complementarity determining regions” or “CDRs”) and/or form structurally defined loops (“hypervariable loops”) and/or contain the antigen-contacting residues (“antigen contacts”). Unless otherwise indicated, CDR residues and other residues in the variable domain (e.g., FR residues) are numbered herein according to Kabat et al., supra. Generally, antibodies comprise six CDRs: three in the VH (H1, H2, H3), and three in the VL (L1, L2, L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including CDR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

An “immunoconjugate” refers to an antibody conjugated to one or more heterologous molecule(s), including but not limited to a cytotoxic agent.

An “isolated” antibody is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” (including references to a specific antibody, e.g., an anti-FGF21 antibody) refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Purified” polypeptide (e.g., antibody), as used herein, refers to a polypeptide that has been increased in purity, such that it exists in a form that is more pure than it exists in its natural environment and/or when initially synthesized and/or amplified under laboratory conditions. Purity is a relative term and does not necessarily mean absolute purity.

The term “package insert,” as used herein, refers to instructions customarily included in commercial packages that contain information concerning the use of the components of the package.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J Immunol. 150:880-887 (1993); Clarkson et al., Nature 352:624-628 (1991).

The terms “host cell,” “host cell line,” and “host cell culture” as used interchangeably herein, refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “label” or “detectable label,” as used herein, refers to any chemical group or moiety that can be linked to a substance that is to be detected or quantitated, e.g., an antibody. A label is a detectable label that is suitable for the sensitive detection or quantification of a substance. Non-limiting examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label may signal its presence by participating in specific binding reactions. Non-limiting examples of such labels include haptens, antibodies, biotin, streptavidin, his-tag, nitrilotriacetic acid, glutathione S-transferase, glutathione and the like.

The term “detection means,” as used herein, refers to a moiety or technique used to detect the presence of the detectable antibody through signal reporting that is then read out in an assay. Typically, a detection means employ reagents, e.g., a detection agent, that amplify an immobilized label such as the label captured onto a microtiter plate, e.g., avidin, streptavidin-HRP or streptavidin-β-D-galactopyranose.

The term “detecting,” is used herein, to include both qualitative and quantitative measurements of a target molecule, e.g., FGF21 or processed forms thereof. In certain embodiments, detecting includes identifying the mere presence of the target molecule in a sample as well as determining whether the target molecule is present in the sample at detectable levels.

An “individual” or “subject,” as used interchangeably herein, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

A “sample,” as used herein, refers to a small portion of a larger quantity of material. In certain embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the sample may be solid tissue (e.g., from a fresh, frozen, and/or preserved organ, tissue sample, biopsy or aspirate), blood or any blood constituents, bodily fluids (such as, e.g., urine, lymph, cerebral spinal fluid, amniotic fluid, peritoneal fluid or interstitial fluid), or cells from the individual, including circulating cells.

### II. Immunoassays

The presently disclosed subject matter provides methods for the detection and quantification of FGF21 protein. In certain embodiments, the present disclosure provides immunoassays for determining the amount of total FGF21 and/or active FGF21 protein in a sample. The present disclosure further provides immunoassay methods for determining the ratio of active FGF21 protein to total FGF21 protein in a sample. In certain embodiments, the immunoassay methods of the present disclosure use the anti-FGF21 antibodies disclosed herein. Non-limiting examples of anti-FGF21 antibodies for use in the presently disclosed methods are provided in Tables 8-13 and 16-19.

In certain embodiments, the present disclosure provides immunoassay methods for the detection and quantification of human FGF21 protein. For example, the immunoassay methods can be used for the detection and quantification of FGF21, e.g., total human FGF21 and/or active human FGF21 protein, in a sample. The immunoassay methods of the present disclosure can incorporate strategies known in the art, including but not limited to, sandwich assay, enzyme-linked immunosorbent assay (ELISA) assay, a digital form of ELISA, electrochemical assay (ECL) assay and magnetic immunoassay. In certain embodiments, the immunoassay method is a single molecule immunoassay, e.g., using a single molecule array. For example, but not by way of limitation, the immunoassay method can be performed using a Quanterix instrument, e.g., a Simoa HD-1 Analyzer™.

In certain embodiments, the methods of the present disclosure comprise contacting a sample obtained from a subject with a capture anti-FGF21 antibody, such as those described herein, under conditions permissive for the binding of the capture anti-FGF21 antibody to FGF21 protein in the sample. For example, but not by way of limitation, the sample can be incubated with a capture antibody that binds to an epitope present on FGF21 to generate a sample-capture antibody combination material. The conditions for the incubation of the sample and the capture antibody can be selected to maximize the sensitivity of the assay and/or to minimize dissociation, as well as to ensure that the FGF21 protein present in the sample binds to the capture antibody.

In certain embodiments, the capture antibodies used in the immunoassay methods disclosed herein can be used at a concentration from about 0.1 μg/ml to about 5.0 μg/ml. For example, but not by way of limitation, the capture antibodies can be used at a concentration from about 0.1 μg/ml to about 0.5 μg/ml, from about 0.1 μg/ml to about 1.0 μg/ml, from about 0.1 μg/ml to about 1.5 μg/ml, from about 0.1 μg/ml to about 2.0 μg/ml, from about 0.1 μg/ml to about 2.5 μg/ml, from about 0.1 μg/ml to about 3.0 μg/ml, from about 0.1 μg/ml to about 3.5 μg/ml, from about 0.1 μg/ml to about 4.0 μg/ml, from about 0.1 μg/ml to about 4.5 μg/ml, from about 0.5 μg/ml to about 5.0 μg/ml, from about 1.0 μg/ml to about 5.0 μg/ml, from about 1.5 μg/ml to about 5.0 μg/ml, from about 2.0 μg/ml to about 5.0 μg/ml, from about 2.5 μg/ml to about 5.0 μg/ml, from about 3.0 μg/ml to about 5.0 μg/ml, from about 3.5 μg/ml to about 5.0 μg/ml, from about 4.0 μg/ml to about 5.0 μg/ml, from about 4.5 μg/ml to about 5.0 μg/ml, from about 0.5 μg/ml to about 2.0 μg/ml or from about 0.5 μg/ml to about 1.0 μg/ml, e.g., about 0.5 μg/ml.

In certain embodiments, the capture antibody can be diluted in a coating buffer. Non-limiting examples of coating buffers include PBS, a carbonate buffer, a bicarbonate buffer or combinations thereof. In certain embodiments, the coating buffer is sodium bicarbonate. In certain embodiments, the coating buffer is PBS. In certain embodiments, the coating buffer can be used at a concentration from about 10 mM to about 1 M. For example, but not by way of limitation, the coating buffer can be used at a concentration from about 10 mM to about 100 mM, from about 10 mM to about 200 mM, from about 10 mM to about 300 mM, from about 10 mM to about 400 mM, from about 10 mM to about 500 mM, from about 10 mM to about 600 mM, from about 10 mM to about 700 mM, from about 10 mM to about 800 mM, from about 10 mM to about 900 mM, from about 100 mM to about 1 M, from about 200 mM to about 1 M, from about 300 mM to about 1 M, from about 400 mM to about 1 M, from about 500 mM to about 1 M, from about 600 mM to about 1 M, from about 700 mM to about 1 M, from about 800 mM to about 1 M or from about 900 mM to about 1 M.

Capture antibodies, as used herein, can be immobilized on a solid phase. For example, but not by way of limitation, the solid phase can be any inert support or carrier that is useful in immunometric assays, including supports in the form of, e.g., surfaces, particles, porous matrices, beads and the like. Non-limiting examples of commonly used supports include small sheets, SEPHADEX®, gels, polyvinyl chloride, plastic beads and assay plates or test tubes manufactured from polyethylene, polypropylene, polystyrene, and the like, including 96-well microtiter plates, as well as particulate materials such as filter paper, agarose, cross-linked dextran, and other polysaccharides. In certain embodiments, the solid phase used for immobilization can be beads. For example, but not by way of limitation, a capture antibody disclosed herein is immobilized to paramagnetic beads. In certain embodiments, the immobilized capture antibodies are coated on a microtiter plate that can be used to analyze several samples at one time.

In certain embodiments, the paramagnetic beads coupled to the capture antibody can be used at a concentration from about 0.1×10⁷ beads/ml to about 10.0×10⁷ beads/ml, e.g., from about 0.1×10⁷ beads/ml to about 0.5×10⁷ beads/ml, from about 0.1×10⁷ beads/ml to about 1.0×10⁷ beads/ml, from about 0.1×10⁷ beads/ml to about 2.0×10⁷ beads/ml, from about 0.1×10⁷ beads/ml to about 3.0×10⁷ beads/ml, from about 0.1×10⁷ beads/ml to about 4.0×10⁷ beads/ml, from about 0.1×10⁷ beads/ml to about 5.0×10⁷ beads/ml, from about 0.1×10⁷ beads/ml to about 6.0×10⁷ beads/ml, from about 0.1×10⁷ beads/ml to about 7.0×10⁷ beads/ml, from about 0.1×10⁷ beads/ml to about 8.0×10⁷ beads/ml, from about 0.1×10⁷ beads/ml to about 9.0×10⁷ beads/ml, from about 0.5×10⁷ beads/ml to about 10.0×10⁷ beads/ml, from about 1.0×10⁷ beads/ml to about 10.0×10⁷ beads/ml, from about 2.0×10⁷ beads/ml to about 10.0×10⁷ beads/ml, from about 3.0×10⁷ beads/ml to about 10.0×10⁷ beads/ml, from about 4.0×10⁷ beads/ml to about 10.0×10⁷ beads/ml, from about 5.0×10⁷ beads/ml to about 10.0×10⁷ beads/ml, from about 6.0×10⁷ beads/ml to about 10.0×10⁷ beads/ml, from about 7.0×10⁷ beads/ml to about 10.0×10⁷ beads/ml, from about 8.0×10⁷ beads/ml to about 10.0×10⁷ beads/ml, from about 9.0×10⁷ beads/ml to about 10.0×10² beads/ml, from about 0.5×10⁷ beads/ml to about 1.0×10⁷ beads/ml, from about 0.5×10⁷ beads/ml to about 2.0×10⁷ beads/ml or from about 0.5×10⁷ beads/ml to about 3.0×10⁷ beads/ml. In certain embodiments, the paramagnetic beds can be used at a concentration from about 0.5×10⁷ beads/ml to about 2.0×10⁷ beads/ml. In certain embodiments, the paramagnetic beds can be used at a concentration of about 1.0×10⁷ beads/ml, e.g., about 1.22×10⁷ beads/ml, or at a concentration of about 0.5×10⁷ beads/ml, e.g., about 0.59×10⁷ beads/ml.

The immunoassay methods disclosed herein can further include contacting a sample-capture antibody combination material with a detector antibody. In certain embodiments, the detector antibody binds to an epitope present on FGF21. In certain embodiments, the detector antibody binds to an epitope present on the sample-capture antibody combination material, but not on the capture antibody in the absence of FGF21. In certain embodiments, the detector antibody bound to the sample-capture antibody combination is subsequently measured or quantified using a detection means, e.g., one or more detection agents, for the detection antibody to determine the amount of FGF21 protein, e.g., total FGF21 or active FGF21 protein, bound by the detector antibody.

In certain embodiments, the detector antibody can be used in a concentration from about 0.1 μg/ml to about 5.0 μg/ml. For example, but not by way of limitation, the detector antibody can be used at a concentration from about 0.1 μg/ml to about 0.5 μg/ml, from about 0.1 μg/ml to about 1.0 μg/ml, from about 0.1 μg/ml to about 1.5 μg/ml, from about 0.1 μg/ml to about 2.0 μg/ml, from about 0.1 μg/ml to about 2.5 μg/ml, from about 0.1 μg/ml to about 3.0 μg/ml, from about 0.1 μg/ml to about 3.5 μg/ml, from about 0.1 μg/ml to about 4.0 μg/ml, from about 0.1 μg/ml to about 4.5 μg/ml, from about 0.5 μg/ml to about 5.0 μg/ml, from about 1.0 μg/ml to about 5.0 μg/ml, from about 1.5 μg/ml to about 5.0 μg/ml, from about 2.0 μg/ml to about 5.0 μg/ml, from about 2.5 μg/ml to about 5.0 μg/ml, from about 3.0 μg/ml to about 5.0 μg/ml, from about 3.5 μg/ml to about 5.0 μg/ml, from about 4.0 μg/ml to about 5.0 μg/ml, from about 4.5 μg/ml to about 5.0 μg/ml, from about 1.0 μg/ml to about 3.0 μg/ml, from about 0.5 μg/ml to about 3.0 μg/ml or from about 0.5 μg/ml to about 2.0 μg/ml. In certain embodiments, an immunoassay for detecting total FGF21 protein can use a detector antibody at a concentration between about 0.1 μg/ml to about 1.0 μg/ml, e.g., about 0.4 μg/ml or about 0.8 μg/ml. In certain embodiments, an immunoassay for detecting active FGF21 protein can use a detector antibody at a concentration between about 1.0 μg/ml to about 3.0 μg/ml, e.g., about 1.1 μg/ml or about 2.1 μg/ml.

In certain embodiments, the anti-FGF21 antibodies for use in the disclosed methods can be labeled. Labels include, but are not limited to, labels or moieties that are detected directly, such as fluorescent, chromophoric, electron-dense, chemiluminescent, and radioactive labels, as well as moieties, such as enzymes or ligands, that are detected indirectly, e.g., through an enzymatic reaction or molecular interaction. Non-limiting examples of labels include the radioisotopes ³²P, ¹⁴C, ¹²⁵I, ³H and ¹³¹I, fluorophores such as rare earth chelates or fluorescein and its derivatives, rhodamine and its derivatives, dansyl, umbelliferone, luciferases, e.g., firefly luciferase and bacterial luciferase (see U.S. Pat. No. 4,737,456), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase or microperoxidase, biotin/avidin, spin labels, bacteriophage labels, stable free radicals and the like. In certain embodiments, the detector antibody is labeled with biotin, e.g., the detector antibody is conjugated to biotin.

In certain embodiments, the detection agent for the biotinylated detector antibody is avidin, streptavidin-HRP or streptavidin-β-D-galactopyranose (SBG). In certain embodiments, the readout of the detection agent is fluorimetric or colorimetric. For example, but not by way of limitation, tetramethylbenzidine and hydrogen peroxide can be used as the readout. In certain embodiments, if the detection agent is streptavidin-HRP, the readout can be colorimetric by using tetramethylbenzidine and hydrogen peroxide. Alternatively, in certain embodiments, resorufin β-D-galactopyranoside can be used as the readout. For example, but not by way of limitation, if the detection agent is SBG, the readout can be fluorimetric by using resorufin β-D-galactopyranoside.

In certain embodiments, the detection agent, e.g., SBG, can be used at a concentration from about 50 to about 500 pM. For example, but not by way of limitation, the detection agent can be used at a concentration from about 50 to about 100 pM, from about 50 to about 150 pM, from about 50 to about 200 pM, from about 50 to about 250 pM, from about 50 to about 300 pM, from about 50 to about 350 pM, from about 50 to about 400 pM, from about 50 to about 450 pM, from about 100 to about 500 pM, from about 150 to about 500 pM, from about 200 to about 500 pM, from about 250 to about 500 pM, from about 300 to about 500 pM, from about 350 to about 500 pM, from about 400 to about 500 pM, from about 450 to about 500 pM, from about 100 to about 400 pM or from about 200 to about 400 pM. In certain embodiments, the detection agent can be used at a concentration from about 100 pM to about 400 pM, e.g., SBG can be used at a concentration of about 110 pM, about 155 pM or about 310 pM. In certain embodiments, SBG is used at a concentration of about 310 pM. In certain embodiments, the detection agent, e.g., HRP, can be used at a dilution from about 1/10 to about 1/1000. For example, but not by way of limitation, the detection agent can be used at a dilution from about 1/10 to about 1/100, from about 1/10 to about 1/500, from about 1/100 to about 1/1000 or from about 1/500 to about 1/1000. In certain embodiments, the detection agent can be used at a dilution from about 1/100 to about 1/1000, e.g., HRP can be used at a dilution of about 1/100 or about 1/500.

In certain embodiments, the methods of the present disclosure can include blocking the capture antibody with a blocking buffer. In certain embodiments, the blocking buffer can include PBS, bovine serum albumin (BSA) and/or a biocide, e.g., ProClin™ (Sigma-Aldrich, Saint Louis, Mo.). In certain embodiments, the method can include multiple washing steps. In certain embodiments, the solution used for washing is generally a buffer (e.g., a “washing buffer”) such as, but not limited to, a PBS buffer that includes a detergent, e.g., Tween 20. For example, but not by way of limitation, the capture antibody can be washed after blocking and/or the sample can be separated from the capture antibody to remove uncaptured material, e.g., by washing.

The immunoassay methods of the present disclosure can be used, in certain embodiments, to detect the amount of total FGF21 protein in a sample, e.g., by detecting full-length and processed forms of FGF21. For example, but not by way of limitation, an immunoassay method for the detection of total FGF21 protein can use one or more antibodies that bind to an epitope present within amino acid residues 5-172 of FGF21, e.g., amino acid residues 5-172 of SEQ ID NO: 1. In certain embodiments, the capture antibody is an antibody that binds to an epitope present within amino acid residues 5-172 of FGF21 and the detector antibody is an antibody that binds to an epitope present within amino acid residues 5-172 of FGF21. In certain embodiments, the capture antibody and the detector antibody are the same antibody, while in other embodiments, the capture antibody and the detector antibody are different antibodies but both bind to an epitope present within amino acid residues 5-172 of FGF21. In certain embodiments, the capture antibody and the detector antibody bind to different epitopes within amino acid residues 5-172 of FGF21. For example, but not by way of limitation, the capture antibody and the detector antibody bind to epitopes within amino acid residues 5-172 of FGF21 that do not overlap. In certain embodiments, the capture antibody and the detector antibody bind to epitopes within amino acid residues 5-172 of FGF21 that partially overlap.

In certain embodiments, an immunoassay for determining the amount of total FGF21 protein in a sample can include (a) contacting a capture antibody that binds to an epitope present within amino acid residues 5-172 of FGF21 with the sample to generate a sample-capture antibody combination material; (b) contacting the sample-capture antibody combination material with a detector antibody that binds to an epitope present within amino acid residues 5-172 of FGF21; (c) detecting the detector antibody bound to the sample-capture antibody combination material; and (d) calculating an amount of total FGF21 protein present in the sample based on the level of the detector antibody bound.

In certain embodiments, an immunoassay method of the present disclosure can be used to detect the amount of active FGF21 protein in a sample, e.g., by detecting FGF21 protein that retains its C-terminal fragment. In certain embodiments, an immunoassay method for the detection of total FGF21 protein can use one or more antibodies that bind to an epitope present within amino acid residues 173-182 of FGF21, e.g., amino acid residues 173-182 of SEQ ID NO: 1, and one or more antibodies that bind to an epitope present within amino acid residues 5-172 of FGF21. For example, but not by way of limitation, an immunoassay method to detect the amount of active FGF21 protein can use a capture antibody that binds to an epitope present within amino acid residues 5-172 of FGF21 and a detector antibody that binds to an epitope present within amino acid residues 173-182 of FGF21. In certain embodiments, the detector antibody that binds to amino acid residues 173-182 of FGF21 can be the anti-FGF21 antibody from Epitope Diagnostics, Inc., San Diego, Calif., sold under catalog number 31002. In certain embodiments, the detector antibody that binds to amino acid residues 173-182 of FGF21 can be the anti-FGF21 antibody from Epitope Diagnostics, Inc., San Diego, Calif., sold under catalog number 30661. In certain embodiments, an immunoassay method for determining the amount of active FGF21 protein in a sample can include (a) contacting a capture antibody that binds to an epitope present within amino acid residues 5-172 of FGF21 with the sample to generate a sample-capture antibody combination material; (b) contacting the sample-capture antibody combination material with a detector antibody that binds to an epitope present within amino acid residues 173-182 of FGF21; (c) detecting the detector antibody bound to the sample-capture antibody combination material; and (d) calculating an amount of active FGF21 protein present in the sample based on the level of the detector antibody bound.

The present disclosure further provides immunoassay methods for determining the ratio of active FGF21 protein to total FGF21 protein in a sample. For example, but not by way of limitation, such methods can involve combining an immunoassay for detecting total FGF21 protein with an immunoassay for detecting active FGF21 protein. In certain embodiments, the immunoassay methods for determining the ratio of active FGF21 protein to total FGF21 protein in a sample can include (a)(i) contacting a first capture antibody that binds to an epitope present within amino acid residues 5-172 of FGF21 with the sample to generate a first sample-capture antibody combination material; (ii) contacting the first sample-capture antibody combination material with a first detector antibody that binds to an epitope present within amino acid residues 5-172 of FGF21; (iii) detecting the first detector antibody bound to the sample-capture antibody combination material; and (iv) calculating an amount of total FGF21 protein present in the sample based on the level of the first detector antibody bound; and (b)(i) contacting a second capture antibody that binds to an epitope present within amino acid residues 5-172 of FGF21 with the sample to generate a second sample-capture antibody combination material; (ii) contacting the second sample-capture antibody combination material with a second detector antibody that binds to an epitope present within amino acid residues 173-182 of FGF21; (iii) detecting the second detector antibody bound to the sample-capture antibody combination material; and (iv) calculating an amount of active FGF21 protein present in the sample based on the level of the second detector antibody bound. The methods can further include comparing the amount of total FGF21 protein as determined by step (a) with the amount of active FGF21 protein as determined by step (b) to determine the ratio of active FGF21 protein to total FGF21 protein in the sample. In certain embodiments, the first capture antibody and second capture antibody are the same antibody. Alternatively, in certain embodiments, the first capture antibody and second capture antibody are different antibodies but both bind to an epitope present within amino acid residues 5-172 of FGF21. In certain embodiments, the first capture antibody and the first detector antibody bind to different epitopes within amino acid residues 5-172 of FGF21. For example, but not by way of limitation, the first capture antibody and the first detector antibody bind to epitopes within amino acid residues 5-172 of FGF21 that do not overlap. In certain embodiments, the first capture antibody and the first detector antibody bind to epitopes within amino acid residues 5-172 of FGF21 that partially overlap.

In certain embodiments, the immunoassay methods disclosed herein have a detection sensitivity, e.g., an in-well sensitivity, from about 2 pg/ml to about 20 pg/ml. For example, but not by way of limitation, an immunoassay disclosed herein has a sensitivity from about 2 pg/ml to about 3 pg/ml, from about 2 pg/ml to about 4 pg/ml, from about 2 pg/ml to about 5 pg/ml, from about 2 pg/ml to about 6 pg/ml, from about 2 pg/ml to about 7 pg/ml, from about 2 pg/ml to about 8 pg/ml, from about 2 pg/ml to about 10 pg/ml, from about 2 pg/ml to about 11 pg/ml, from about 2 pg/ml to about 12 pg/ml, from about 2 pg/ml to about 13 pg/ml, from about 2 pg/ml to about 14 pg/ml, from about 2 pg/ml to about 15 pg/ml, from about 2 pg/ml to about 16 pg/ml, from about 2 pg/ml to about 17 pg/ml, from about 2 pg/ml to about 18 pg/ml, from about 2 pg/ml to about 19 pg/ml, from about 3 pg/ml to about 15 pg/ml, from about 3 pg/ml to about 10 pg/ml or from about 3 pg/ml to about 5 pg/ml. In certain embodiments, an immunoassay disclosed herein has a sensitivity of about 2 pg/ml or greater, 1 pg/ml or greater or 0.5 pg/ml or greater. In certain embodiments, an immunoassay disclosed herein has a detection sensitivity, e.g., an in-well sensitivity, from about 0.2 pg/ml to about 2.0 pg/ml, e.g., from about 0.2 pg/ml to about 0.5 pg/ml, from about 0.2 pg/ml to about 1.0 pg/ml or from about 0.2 pg/ml to about 1.5 pg/ml. For example, but not by way of limitation, an immunoassay disclosed herein, e.g., a single molecule immunoassay using the Simoa HD-1 Analyzer™, has a sensitivity, e.g., an in-well sensitivity, from about 0.2 pg/ml to about 0.5 pg/ml.

The samples analyzed by the immunoassay methods of the present disclosure can be clinical samples, cells in culture, cell supernatants, cell lysates, serum samples, blood plasma samples, other biological fluid (e.g., lymphatic fluid) samples or tissue samples. In certain embodiments, the source of the sample may be solid tissue (e.g., from a fresh, frozen and/or preserved organ, tissue sample, serum, blood plasma, biopsy or aspirate) or cells from a subject. In certain embodiments, the sample is a blood sample. In certain embodiments, the sample is a plasma sample. In certain embodiments, the sample, e.g., blood or plasma sample, can be obtained from a subject and treated with one or more protease, esterase, DDP-IV and/or phosphatase inhibitors. For example, but not by way of limitation, a sample can be treated with a cocktail of protease and phosphatase inhibitors, e.g., MS-SAFE (Sigma-Aldrich, Saint Louis, Mo.). In certain embodiments, the sample is treated with an anti-coagulant or collected in tube that contains an anti-coagulant, e.g., K₂-EDTA. In certain embodiments, the sample can be collected using the P800 Blood Collection System (BD Biosciences, San Jose, Calif.).

### III. Antibodies

The present disclosure further provides antibodies that bind to FGF21, e.g., human FGF21. Antibodies of the present disclosure are useful for detecting and quantifying FGF21 protein levels in a sample. In certain embodiments, the antibodies of the present disclosure can be used in immunoassay methods for the detection and quantification of FGF21 protein, disclosed herein. For example, but not by way of limitation, antibodies of the present disclosure can be used to detect the levels of total FGF21 protein and/or active FGF21 protein in a sample.

In certain embodiments, an antibody of the present disclosure can be humanized. In certain embodiments, an antibody of the present disclosure comprises an acceptor human framework, e.g., a human immunoglobulin framework or a human consensus framework. In certain embodiments, an antibody of the present disclosure can be a monoclonal antibody, including a chimeric, humanized or human antibody. For example, but not by way of limitation, an antibody of the present disclosure can be chimeric. In certain embodiments, an antibody of the present disclosure can be an antibody fragment, e.g., a Fv, Fab, Fab′, scFv, diabody or F(ab′)₂ fragment. In certain embodiments, the antibody is an IgG. In certain embodiments, the antibody is selected from IgG1, IgG2, IgG3 and IgG4. In certain embodiments, the antibody is a full-length antibody, e.g., an intact IgG1 antibody, or other antibody class or isotype as defined herein. In certain embodiments, the anti-FGF21 antibodies disclosed herein can be labeled, e.g., conjugated to biotin. In certain embodiments, an antibody of the present disclosure can incorporate any of the features, singly or in combination, as described in Sections 1-7, detailed below.

A. Exemplary Anti-FGF21 Antibodies

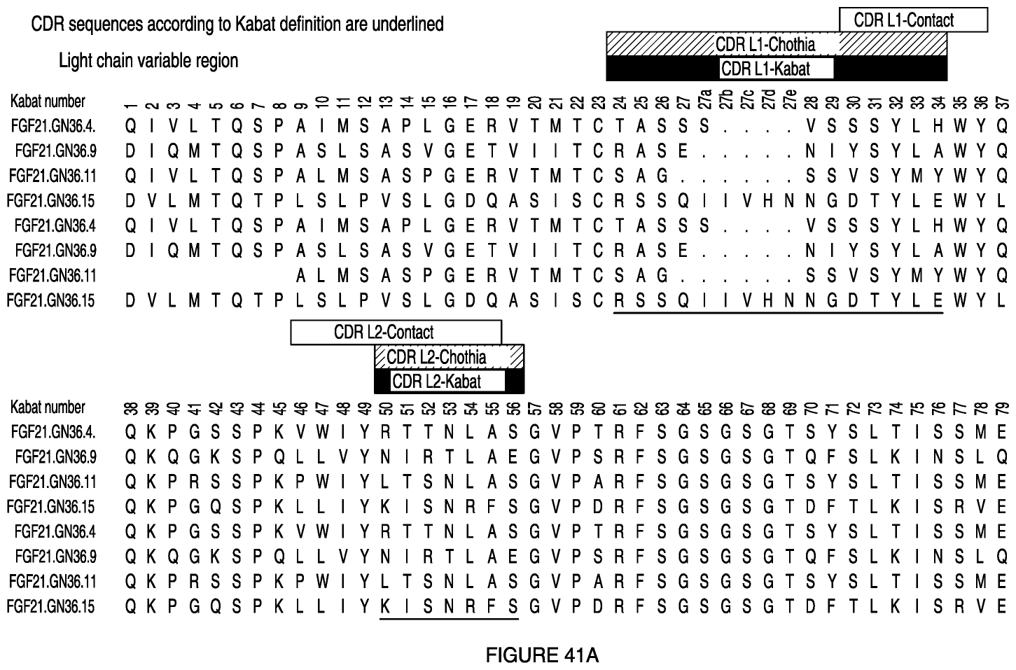

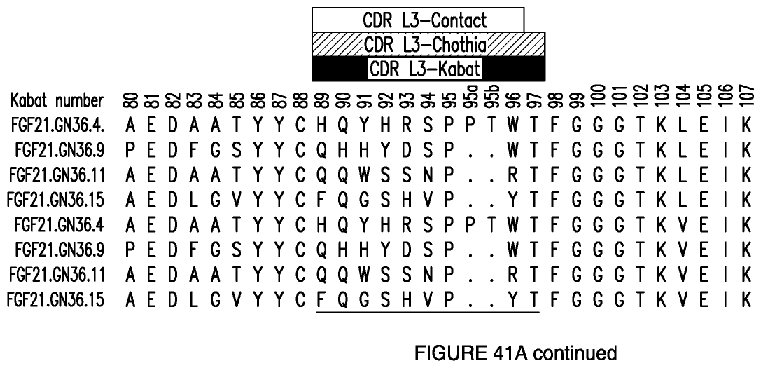

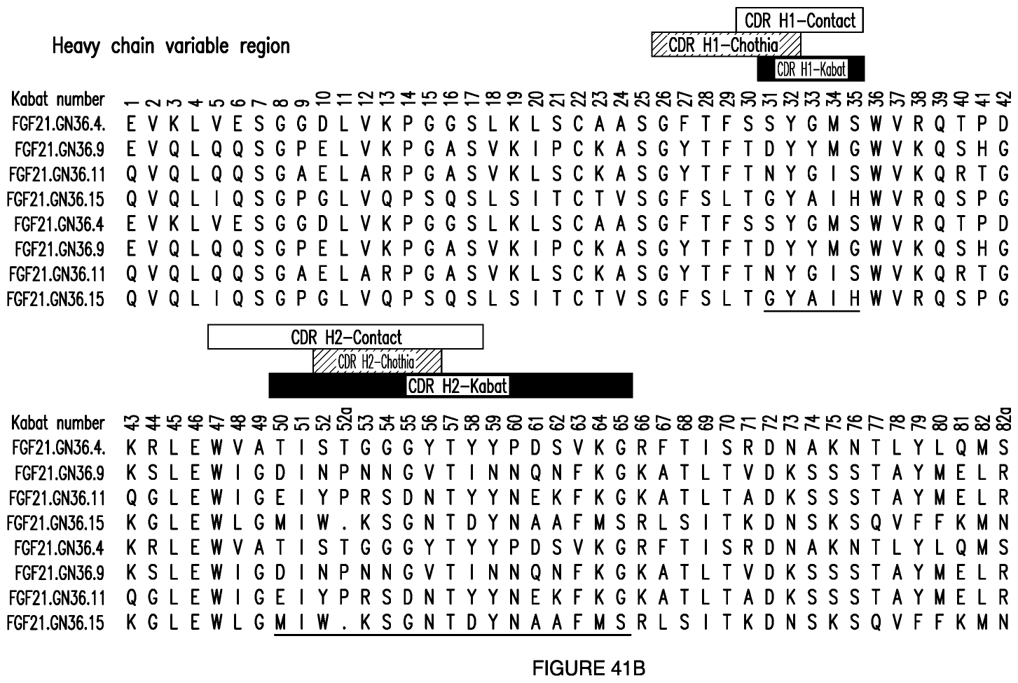

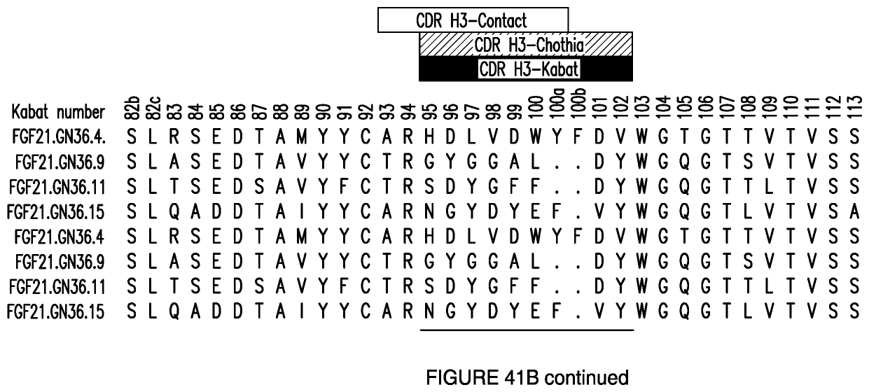

The present disclosure provides isolated antibodies that bind to a FGF21 protein. In certain embodiments, an antibody of the present disclosure can bind to an epitope present within amino acid residues 5-172 of FGF21, e.g., amino acid residues 5-172 of SEQ ID NO: 1. In certain embodiments, an antibody of the present disclosure can bind to an epitope present within amino acid residues 173-182 of FGF21, e.g., amino acid residues 173-182 of SEQ ID NO: 1. In certain embodiments, an antibody of the present disclosure does not bind to an epitope present within amino acid residues 1-4 of FGF21, e.g., amino acid residues 1-4 of SEQ ID NO: 1. Non-limiting examples of anti-FGF21 antibodies are disclosed in Tables 8-13 and 16-19 and FIG. 41A-B.

The present disclosure provides anti-FGF21 antibodies comprising, in certain embodiments, at least one, two, three, four, five or six CDRs selected from (a) CDR-H1 comprising an amino acid sequence of any one of SEQ ID NOs: 26-29 and conservative substitutions thereof; (b) CDR-H2 comprising an amino acid sequence of any one of SEQ ID NOs: 30-33 and conservative substitutions thereof; (c) CDR-H3 comprising an amino acid sequence of any one of SEQ ID NOs: 34-37 and conservative substitutions thereof; (d) CDR-L1 comprising an amino acid sequence of any one of SEQ ID NOs: 38-41 and conservative substitutions thereof; (e) CDR-L2 comprising SEQ ID NOs: 42-45 and conservative substitutions thereof; and (f) CDR-L3 comprising an amino acid sequence of any one of SEQ ID NOs: 46-49 and conservative substitutions thereof.

The present disclosure provides anti-FGF21 antibodies that, in certain embodiments, comprise: (a) CDR-H1 comprising the amino acid sequence of SEQ ID NO: 26 and conservative substitutions thereof; (b) CDR-H2 comprising the amino acid sequence of SEQ ID NO: 30 and conservative substitutions thereof; (c) CDR-H3 comprising the amino acid sequence of SEQ ID NO: 34 and conservative substitutions thereof; (d) CDR-L1 comprising the amino acid sequence of SEQ ID NO: 38 and conservative substitutions thereof; (e) CDR-L2 comprising the amino acid sequence of SEQ ID NO: 42 and conservative substitutions thereof; and (f) CDR-L3 comprising the amino acid sequence of SEQ ID NO: 46 and conservative substitutions thereof.

The present disclosure provides anti-FGF21 antibodies that, in certain embodiments, comprise: (a) CDR-H1 comprising the amino acid sequence of SEQ ID NO: 27 and conservative substitutions thereof; (b) CDR-H2 comprising the amino acid sequence of SEQ ID NO: 31 and conservative substitutions thereof; (c) CDR-H3 comprising the amino acid sequence of SEQ ID NO: 35; (d) CDR-L1 comprising the amino acid sequence of SEQ ID NO: 39 and conservative substitutions thereof; (e) CDR-L2 comprising the amino acid sequence of SEQ ID NO: 43 and conservative substitutions thereof; and (f) CDR-L3 comprising the amino acid sequence of SEQ ID NO: 47 and conservative substitutions thereof.

The present disclosure provides anti-FGF21 antibodies that, in certain embodiments, comprise: (a) CDR-H1 comprising the amino acid sequence of SEQ ID NO: 28 and conservative substitutions thereof; (b) CDR-H2 comprising the amino acid sequence of SEQ ID NO: 32 and conservative substitutions thereof; (c) CDR-H3 comprising the amino acid sequence of SEQ ID NO: 36 and conservative substitutions thereof; (d) CDR-L1 comprising the amino acid sequence of SEQ ID NO: 40 and conservative substitutions thereof; (e) CDR-L2 comprising the amino acid sequence of SEQ ID NO: 44 and conservative substitutions thereof; and (f) CDR-L3 comprising the amino acid sequence of SEQ ID NO: 48 and conservative substitutions thereof.

The present disclosure provides anti-FGF21 antibodies that, in certain embodiments, comprise: (a) CDR-H1 comprising the amino acid sequence of SEQ ID NO: 29 and conservative substitutions thereof; (b) CDR-H2 comprising the amino acid sequence of SEQ ID NO: 33 and conservative substitutions thereof; (c) CDR-H3 comprising the amino acid sequence of SEQ ID NO: 37 and conservative substitutions thereof; (d) CDR-L1 comprising the amino acid sequence of SEQ ID NO: 41 and conservative substitutions thereof; (e) CDR-L2 comprising the amino acid sequence of SEQ ID NO: 45 and conservative substitutions thereof; and (f) CDR-L3 comprising the amino acid sequence of SEQ ID NO: 49 and conservative substitutions thereof.

In certain embodiments, an anti-FGF21 antibody of the present disclosure comprises a heavy chain variable domain (VH) sequence having at least 90%, 91%, 92%, 93%, 94%, 95%, 96%, 97%, 98%, 99% or 100% sequence identity to an amino acid sequence of any one of SEQ ID NOs: 54-57 and 72-75. In certain embodiments, a VH sequence having at least 90%, 91%, 92%, 93%, 94%, 95%, 96%, 97%, 98% or 99% identity contains substitutions (e.g., conservative substitutions), insertions or deletions relative to the reference sequence, but an anti-FGF21 antibody comprising that sequence retains the ability to bind to FGF21. In certain embodiments, a total of 1 to 10 amino acids have been substituted, inserted and/or deleted. In certain embodiments, substitutions, insertions or deletions occur in regions outside the CDRs (i.e., in the FRs). In certain embodiments, an anti-FGF21 antibody of the present disclosure comprises a VH sequence comprising an amino acid sequence of any one of SEQ ID NOs: 54-57 and 72-75.

In certain embodiments, an anti-FGF21 antibody of the present disclosure comprises a light chain variable domain (VL) sequence having at least 90%, 91%, 92%, 93%, 94%, 95%, 96%, 97%, 98%, 99% or 100% sequence identity to an amino acid sequence of anyone of SEQ ID NOs: 50-53 and 68-71. In certain embodiments, a VL sequence having at least 90%, 91%, 92%, 93%, 94%, 95%, 96%, 97%, 98% or 99% identity contains substitutions (e.g., conservative substitutions), insertions or deletions relative to the reference sequence, but an anti-FGF21 antibody comprising that sequence retains the ability to bind to FGF21. In certain embodiments, a total of 1 to 10 amino acids have been substituted, inserted and/or deleted. In certain embodiments, substitutions, insertions or deletions occur in regions outside the CDRs (i.e., in the FRs). In certain embodiments, an anti-FGF21 antibody of the present disclosure comprises a VL sequence comprising an amino acid sequence of any one of SEQ ID NOs: 50-53 and 68-71.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 54. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 50. In certain embodiments, the VH comprises one, two or three CDRs selected from: (a) CDR-H1 comprising the amino acid sequence of SEQ ID NO: 26, (b) CDR-H2 comprising the amino acid sequence of SEQ ID NO: 30, and (c) CDR-H3 comprising the amino acid sequence of SEQ ID NO: 34. In certain embodiments, the VL comprises one, two or three CDRs selected from: (a) CDR-L1 comprising the amino acid sequence of SEQ ID NO: 38, (b) CDR-L2 comprising the amino acid sequence of SEQ ID NO: 42, and (c) CDR-L3 comprising the amino acid sequence of SEQ ID NO: 46.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 55. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 51. In certain embodiments, the VH comprises one, two or three CDRs selected from: (a) CDR-H1 comprising the amino acid sequence of SEQ ID NO: 27, (b) CDR-H2 comprising the amino acid sequence of SEQ ID NO: 31, and (c) CDR-H3 comprising the amino acid sequence of SEQ ID NO: 35. In certain embodiments, the VL comprises one, two or three CDRs selected from: (a) CDR-L1 comprising the amino acid sequence of SEQ ID NO: 39, (b) CDR-L2 comprising the amino acid sequence of SEQ ID NO: 43, and (c) CDR-L3 comprising the amino acid sequence of SEQ ID NO: 47.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 56. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 52. In certain embodiments, the VH comprises one, two or three CDRs selected from: (a) CDR-H1 comprising the amino acid sequence of SEQ ID NO: 28, (b) CDR-H2 comprising the amino acid sequence of SEQ ID NO: 32, and (c) CDR-H3 comprising the amino acid sequence of SEQ ID NO: 36. In certain embodiments, the VL comprises one, two or three CDRs selected from: (a) CDR-L1 comprising the amino acid sequence of SEQ ID NO: 40, (b) CDR-L2 comprising the amino acid sequence of SEQ ID NO: 44, and (c) CDR-L3 comprising the amino acid sequence of SEQ ID NO: 48.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 57. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 53. In certain embodiments, the VH comprises one, two or three CDRs selected from: (a) CDR-H1 comprising the amino acid sequence of SEQ ID NO: 29, (b) CDR-H2 comprising the amino acid sequence of SEQ ID NO: 33, and (c) CDR-H3 comprising the amino acid sequence of SEQ ID NO: 37. In certain embodiments, the VL comprises one, two or three CDRs selected from: (a) CDR-L1 comprising the amino acid sequence of SEQ ID NO: 41, (b) CDR-L2 comprising the amino acid sequence of SEQ ID NO: 45, and (c) CDR-L3 comprising the amino acid sequence of SEQ ID NO: 49.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 75. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 71. In certain embodiments, the VH comprises one, two or three CDRs selected from: (a) CDR-H1 comprising the amino acid sequence of SEQ ID NO: 26, (b) CDR-H2 comprising the amino acid sequence of SEQ ID NO: 30, and (c) CDR-H3 comprising the amino acid sequence of SEQ ID NO: 34. In certain embodiments, the VL comprises one, two or three CDRs selected from: (a) CDR-L1 comprising the amino acid sequence of SEQ ID NO: 38, (b) CDR-L2 comprising the amino acid sequence of SEQ ID NO: 42, and (c) CDR-L3 comprising the amino acid sequence of SEQ ID NO: 46.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 74. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 70. In certain embodiments, the VH comprises one, two or three CDRs selected from: (a) CDR-H1 comprising the amino acid sequence of SEQ ID NO: 27, (b) CDR-H2 comprising the amino acid sequence of SEQ ID NO: 31, and (c) CDR-H3 comprising the amino acid sequence of SEQ ID NO: 35. In certain embodiments, the VL comprises one, two or three CDRs selected from: (a) CDR-L1 comprising the amino acid sequence of SEQ ID NO: 39, (b) CDR-L2 comprising the amino acid sequence of SEQ ID NO: 43, and (c) CDR-L3 comprising the amino acid sequence of SEQ ID NO: 47.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 73. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 69. In certain embodiments, the VH comprises one, two or three CDRs selected from: (a) CDR-H1 comprising the amino acid sequence of SEQ ID NO: 28, (b) CDR-H2 comprising the amino acid sequence of SEQ ID NO: 32, and (c) CDR-H3 comprising the amino acid sequence of SEQ ID NO: 36. In certain embodiments, the VL comprises one, two or three CDRs selected from: (a) CDR-L1 comprising the amino acid sequence of SEQ ID NO: 40, (b) CDR-L2 comprising the amino acid sequence of SEQ ID NO: 44, and (c) CDR-L3 comprising the amino acid sequence of SEQ ID NO: 48.

In certain embodiments, an anti-FGF21 antibody of the present disclosure comprises a VH sequence having at least 90%, 91%, 92%, 93%, 94%, 95%, 96%, 97%, 98%, 99% or 100% sequence identity to the amino acid sequence of SEQ ID NO: 72. In certain embodiments, an anti-FGF21 antibody of the present disclosure comprises a VL sequence having at least 90%, 91%, 92%, 93%, 94%, 95%, 96%, 97%, 98%, 99% or 100% sequence identity to the amino acid sequence of SEQ ID NO: 68. In certain embodiments, the VH comprises one, two or three CDRs selected from: (a) CDR-H1 comprising the amino acid sequence of SEQ ID NO: 29, (b) CDR-H2 comprising the amino acid sequence of SEQ ID NO: 33, and (c) CDR-H3 comprising the amino acid sequence of SEQ ID NO: 37. In certain embodiments, the VL comprises one, two or three CDRs selected from: (a) CDR-L1 comprising the amino acid sequence of SEQ ID NO: 41, (b) CDR-L2 comprising the amino acid sequence of SEQ ID NO: 45, and (c) CDR-L3 comprising the amino acid sequence of SEQ ID NO: 49.

In certain embodiments, an anti-FGF21 antibody of the present disclosure comprises a full-length heavy chain (HC) sequence having at least 90%, 91%, 92%, 93%, 94%, 95%, 96%, 97%, 98%, 99% or 100% sequence identity to an amino acid sequence of any one of SEQ ID NOs: 22-25 and 64-67. In certain embodiments, a HC sequence having at least 90%, 91%, 92%, 93%, 94%, 95%, 96%, 97%, 98% or 99% identity contains substitutions (e.g., conservative substitutions), insertions or deletions relative to the reference sequence, but an anti-FGF21 antibody comprising that sequence retains the ability to bind to FGF21. In certain embodiments, a total of 1 to 10 amino acids have been substituted, inserted and/or deleted. In certain embodiments, substitutions, insertions or deletions occur in regions outside the CDRs (i.e., in the FRs). In certain embodiments, an anti-FGF21 antibody of the present disclosure comprises a HC sequence comprising an amino acid sequence of any one of SEQ ID NOs: 22-25 and 64-67.

In certain embodiments, an anti-FGF21 antibody of the present disclosure comprises a full-length light chain (LC) sequence having at least 90%, 91%, 92%, 93%, 94%, 95%, 96%, 97%, 98%, 99% or 100% sequence identity to an amino acid sequence of any one of SEQ ID NOs: 18-21 and 60-63. In certain embodiments, a LC sequence having at least 90%, 91%, 92%, 93%, 94%, 95%, 96%, 97%, 98% or 99% identity contains substitutions (e.g., conservative substitutions), insertions or deletions relative to the reference sequence, but an anti-FGF21 antibody comprising that sequence retains the ability to bind to FGF21. In certain embodiments, a total of 1 to 10 amino acids have been substituted, inserted and/or deleted. In certain embodiments, substitutions, insertions or deletions occur in regions outside the CDRs (i.e., in the FRs). In certain embodiments, an anti-FGF21 antibody of the present disclosure comprises a LC sequence comprising an amino acid sequence of any one of SEQ ID NOs: 18-21 and 60-63.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 22. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 18.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 23. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 19.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 24. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 20.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 25. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 21.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 67. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 63.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 66. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 62.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 65. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 61.

In certain embodiments, an anti-FGF21 antibody of the present disclosure comprises a HC sequence having at least 90%, 91%, 92%, 93%, 94%, 95%, 96%, 97%, 98%, 99% or 100% sequence identity to the amino acid sequence of SEQ ID NO: 64. In certain embodiments, an anti-FGF21 antibody of the present disclosure comprises a LC sequence having at least 90%, 91%, 92%, 93%, 94%, 95%, 96%, 97%, 98%, 99% or 100% sequence identity to the amino acid sequence of SEQ ID NO: 60.

In certain embodiments, an anti-FGF21 antibody is provided, wherein the antibody comprises a VH as in any of the embodiments provided above, and a VL as in any of the embodiments provided above. In certain embodiments, an anti-FGF21 antibody is provided, wherein the antibody comprises a full-length HC as in any of the embodiments provided above, and a full-length LC as in any of the embodiments provided above.

1. Antibody Affinity

In certain embodiments, an anti-FGF21 antibody of the present disclosure can have a dissociation constant (K_{d}) of ≤1 M, ≤100 mM, ≤10 mM, ≤1 mM, ≤100 μM, ≤10 μM, ≤1 μM, ≤100 nM, ≤10 nM, ≤1 nM, ≤0.1 nM, ≤0.01 nM or ≤0.001 nM. In certain embodiments, an antibody of the present disclosure can have a K_{d} of about 10⁻³ or less or 10⁻⁸ M or less, e.g., from 10⁻⁸ M to 10⁻¹³ M, e.g., from 10⁻⁹ M to 10⁻¹³ M. In certain embodiments, an anti-FGF21 antibody, disclosed herein, can have a K_{d} of about 10⁻¹⁰ M to 10⁻¹³ M. For example, but not by way of limitation, a capture antibody or a detector antibody of the present disclosure binds to FGF21 with a K_{d} from about 10⁻¹⁰ M to 10⁻¹³ M.

In certain embodiments, K_{d} can be measured by a radiolabeled antigen binding assay (RIA). In certain embodiments, an RIA can be performed with a Fab version of an antibody of interest and its antigen. For example, but not by way of limitation, a solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol. Biol. 293:865-881(1999)). To establish conditions for the assay, MICROTITER multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

In certain embodiments, K_{d} can be measured using a BIACORE® surface plasmon resonance assay. For example, but not by way of limitation, an assay using a BIACORE©-2000, a BIACORE©-3000, a BIACORE X100 or a BIACORE T200 processing unit (Biacore, Inc., Piscataway, N.J.) is performed at 25° C. with immobilized antigen CM5 chips at ˜10 response units (RU). In certain embodiments, carboxymethylated dextran biosensor chips (CM5, Biacore, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE© Evaluation Software version 3.2) by simultaneously fitting the association and dissociation sensorgrams. The equilibrium dissociation constant (K_{d}) can be calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody of the present disclosure is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For a discussion of Fab and F(ab′)₂ fragments comprising salvage receptor binding epitope residues and having increased in vivo half-life, see U.S. Pat. No. 5,869,046.

In certain embodiments, an antibody of the present disclosure can be a diabody. Diabodies are antibody fragments comprising two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies, which are additional antibody fragments within the scope of the antibodies of the present disclosure, are also described in Hudson et al., Nat. Med. 9:129-134 (2003).

In certain embodiments, an antibody of the present disclosure can be a single-domain antibody. Single-domain antibodies are antibody fragments that comprise all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques including, but not limited to, proteolytic digestion of an intact antibody as well as production by recombinant host cells (e.g., E. coli or phage), as described herein.

3. Chimeric and Humanized Antibodies

In certain embodiments, an antibody of the present disclosure is a chimeric antibody. Certain chimeric antibodies are described in the art, e.g., in U.S. Pat. No. 4,816,567; and Morrison et al., Proc. Natl. Acad. Sci. USA, 81:6851-6855 (1984)). In certain embodiments, a chimeric antibody of the present disclosure comprises a non-human variable region (e.g., a variable region derived from a mouse, rat, hamster, rabbit or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In certain embodiments, a chimeric antibody of the present disclosure can be a humanized antibody. Typically, a non-human antibody is humanized to reduce immunogenicity to humans, while retaining the specificity and affinity of the parental non-human antibody. Generally, a humanized antibody comprises one or more variable domains in which CDRs, e.g., CDRs, (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J Biol. Chem. 271:22611-22618 (1996)).

4. Human Antibodies

In certain embodiments, an antibody of the present disclosure can be a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boemer et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the present disclosure can be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (e.g., from human) to provide a single source of antibodies to a wide range of non-self and also self antigens without any immunization as described by Griffiths et al., EMBO J, 12: 725-734 (1993). In certain embodiments, naïve libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody of the present disclosure can be a multispecific antibody, e.g., a bispecific antibody. Multispecific antibodies are monoclonal antibodies that have binding specificities for at least two different epitopes. In certain embodiments, one of the binding specificities is for an epitope present on FGF21 and the other is for any other antigen. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J Immunol., 152:5368 (1994)); and preparing trispecific antibodies as described, e.g., in Tutt et al. J Immunol. 147: 60 (1991).

Engineered antibodies with three or more functional antigen binding sites, including “Octopus antibodies,” are also included herein (see, e.g., US 2006/0025576A1).

7. Antibody Variants

The presently disclosed subject matter further provides amino acid sequence variants of the disclosed antibodies. For example, it may be desirable to improve the binding affinity and/or other biological properties of the antibody. Amino acid sequence variants of an antibody can be prepared by introducing appropriate modifications into the nucleotide sequence encoding the antibody or by peptide synthesis. Such modifications include, but are not limited to, deletions from, and/or insertions into and/or substitutions of residues within the amino acid sequences of the antibody. Any combination of deletion, insertion, and substitution can be made to arrive at the final construct, provided that the final antibody, i.e., modified, possesses the desired characteristics, e.g., antigen-binding.

a) Substitution, Insertion, and Deletion Variants

Antibody variants can have one or more amino acid substitutions, insertions and/or deletions. Sites of interest for such variation include, but are not limited to, the CDRs, and FRs. Non-limiting examples of conservative substitutions are shown in Table 1 under the heading of “preferred substitutions.” Non-limiting examples of more substantial changes are provided in Table 1 under the heading of “exemplary substitutions,” and as further described below in reference to amino acid side chain classes. Amino acid substitutions can be introduced into an antibody of interest and the products screened for a desired activity, e.g., retained/improved antigen binding, decreased immunogenicity or improved complement dependent cytotoxicity (CDC) or antibody-dependent cell-mediated cytotoxicity (ADCC).

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

In certain embodiments, non-conservative substitutions will entail exchanging a member of one of these classes for another class.

In certain embodiments, a type of substitutional variant involves substituting one or more hypervariable region residues of a parent antibody, e.g., a humanized or human antibody. Generally, the resulting variant(s) selected for further study will have modifications, e.g., improvements, in certain biological properties such as, but not limited to, increased affinity, reduced immunogenicity, relative to the parent antibody and/or will have substantially retained certain biological properties of the parent antibody. A non-limiting example of a substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g., binding affinity).

In certain embodiments, alterations (e.g., substitutions) can be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)). In certain embodiments of affinity maturation, diversity can be introduced into the variable genes chosen for maturation by any of a variety of methods (e.g., error-prone PCR, chain shuffling or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding can be specifically identified, e.g., using alanine scanning mutagenesis or modeling. CDR-H3 and CDR-L3 in particular are often targeted.

In certain embodiments, substitutions, insertions and/or deletions can occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for Antibody-directed enzyme prodrug therapy (ADEPT)) or a polypeptide which increases the serum half-life of the antibody.

b) Glcosylation variants

Antibodies of the present disclosure can, in certain embodiments, be altered to increase or decrease the extent to which the antibody is glycosylated. For example, but not by way of limitation, the addition or deletion of glycosylation sites of an antibody may be conveniently accomplished by altering the amino acid sequence such that one or more glycosylation sites is created or removed.

Where the antibodies of the present disclosure comprise an Fc region, the carbohydrate attached thereto, if present, can be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In certain embodiments, modifications of the oligosaccharide in an antibody of the present disclosure can be made in order to create antibody variants with certain improved properties.

In certain embodiments, antibody variants are provided having a carbohydrate structure that lacks fucose attached (directly or indirectly) to an Fc region. For example, the amount of fucose in such antibody can be from about 1% to about 80%, from about 1% to about 65%, from about 5% to about 65% or from about 20% to about 40% and values in between.

In certain embodiments, the amount of fucose can be determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 can also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004).

Defucosylated antibodies can be produced in any cell line that are deficient in protein fucosylation. Non-limiting examples of cell lines include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Non-limiting examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants can have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications can be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the present disclosure provides antibody variants that possess some but not all effector functions. Such limited effector function can make the antibody variants desirable candidates for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J Exp. Med. 166:1351-1361 (1987)). Alternatively, non-radioactive assays methods can be employed (see, for example, ACTI™ non-radioactive cytotoxicity assay for flow cytometry (Cell Technology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively or additionally, ADCC activity of the molecule of interest may be assessed in vivo, e.g., in an animal model such as that disclosed in Clynes et al. Proc. Nat'l Acad. Sci. USA 95:652-656 (1998). C1 q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)). In certain embodiments, alterations can be made in the Fc region that result in altered (i.e., either improved or diminished) C1 q binding and/or Complement Dependent Cytotoxicity (CDC), e.g., as described in U.S. Pat. No. 6,194,551, WO 99/51642, and Idusogie et al. J Immunol. 164: 4178-4184 (2000).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. See, e.g., U.S. Pat. No. 6,737,056; WO 2004/056312, and Shields et al., J. Biol. Chem. 9(2): 6591-6604 (2001).

In certain embodiments, antibody variants of the present disclosure comprise an Fc region with one or more amino acid substitutions that improve ADCC, e.g., substitutions at positions 298, 333, and/or 334 of the Fc region (EU numbering of residues).

In certain embodiments, alteration made in the Fc region of an antibody, e.g., a bispecific antibody, disclosed herein, can produce a variant antibody with an increased half-life and improved binding to the neonatal Fc receptor (FcRn), which is responsible for the transfer of maternal IgGs to the fetus (Guyer et al., J Immunol. 117:587 (1976) and Kim et al., J Immunol. 24:249 (1994)), are described in US2005/0014934A1 (Hinton et al.). Those antibodies comprise an Fc region with one or more substitutions therein, which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies can be generated as described, e.g., in U.S. Pat. No. 7,521,541.

e) Antibody Derivatives

In certain embodiments, antibodies of the present disclosure can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). In certain embodiments, the radiation can be of any wavelength, and includes, but is not limited to, wavelengths that do not harm ordinary cells, but which heat the nonproteinaceous moiety to a temperature at which cells proximal to the antibody-nonproteinaceous moiety are killed.

B. Methods of Antibody Production

The antibodies disclosed herein can be produced using any available or known technique in the art. For example, but not by way of limitation, antibodies can be produced using recombinant methods and compositions, e.g., as described in U.S. Pat. No. 4,816,567. Detailed procedures to generate antibodies are described in the Examples below.

The presently disclosed subject matter further provides an isolated nucleic acid encoding an antibody disclosed herein. For example, the isolated nucleic acid can encode an amino acid sequence that includes the VL and/or an amino acid sequence comprising the VH of the antibody, e.g., the light and/or heavy chains of the antibody. In certain embodiments, the isolated nucleic acid can include a nucleotide sequence that encodes a heavy chain variable region amino acid sequence having the sequence set forth in SEQ ID NO: 54, and/or a nucleotide sequence that encodes a light chain variable region amino acid sequence having the sequence set forth in SEQ ID NO: 50. In certain embodiments, the isolated nucleic acid can include a nucleotide sequence that encodes a heavy chain variable region amino acid sequence having the sequence set forth in SEQ ID NO: 57, and/or a nucleotide sequence that encodes a light chain variable region amino acid sequence having the sequence set forth in SEQ ID NO: 53.

In certain embodiments, the nucleic acid can be present in one or more vectors, e.g., expression vectors. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the disclosed subject matter is intended to include such other forms of expression vectors, such as viral vectors (e.g., replication defective retroviruses, adenoviruses and adeno-associated viruses) that serve equivalent functions.

In certain embodiments, the nucleic acid encoding an antibody of the present disclosure and/or the one or more vectors including the nucleic acid can be introduced into a host cell. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, a host cell can include, e.g., has been transformed with: (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In certain embodiments, the host cell is eukaryotic, e.g., a Chinese Hamster Ovary (CHO) cell or lymphoid cell (e.g., Y0, NS0, Sp20 cell).

In certain embodiments, the methods of making a disclosed anti-FGF21 antibody can include culturing a host cell, in which a nucleic acid encoding the antibody has been introduced, under conditions suitable for expression of the antibody, and optionally recovering the antibody from the host cell and/or host cell culture medium. In certain embodiments, the antibody is recovered from the host cell through chromatography techniques.

For recombinant production of an antibody of the present disclosure, a nucleic acid encoding an antibody, e.g., as described above, can be isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies can be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006). Suitable host cells for the expression of glycosylated antibody can also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

In certain embodiments, plant cell cultures can be utilized as host cells. See, e.g., U.S. Pat. Nos. 5,959,177, 6,040,498, 6,420,548, 7,125,978, and 6,417,429 (describing PLANTIBODIES™ technology for producing antibodies in transgenic plants).

In certain embodiments, vertebrate cells can also be used as hosts. For example, but not by way of limitation, mammalian cell lines that are adapted to grow in suspension can be useful. Non-limiting examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In certain embodiments, techniques for making bispecific and/or multispecific antibodies include, but are not limited to, recombinant co-expression of two immunoglobulin heavy chain-light chain pairs having different specificities (see Milstein and Cuello, Nature 305: 537 (1983)), PCT Patent Application No. WO 93/08829, and Traunecker et al., EMBO J. 10: 3655 (1991)), and “knob-in-hole” engineering (see, e.g., U.S. Pat. No. 5,731,168). Bispecific antibodies can also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bispecific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J Immunol., 152:5368 (1994)); and preparing trispecific antibodies as described, e.g., in Tutt et al. J Immunol. 147: 60 (1991).

Bispecific and multispecific molecules of the present disclosure can also be made using chemical techniques (see, e.g., Kranz (1981) Proc. Natl. Acad. Sci. USA 78:5807), “polydoma” techniques (see, e.g., U.S. Pat. No. 4,474,893) or recombinant DNA techniques. Bispecific and multispecific molecules of the presently disclosed subject matter can also be prepared by conjugating the constituent binding specificities, e.g., a first epitope and a second epitope binding specificities, using methods known in the art and as described herein. For example, but not by way of limitation, each binding specificity of the bispecific and multispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), N-succinimidyl-3-(2-pyridyldithio)propionate (SPDP), and sulfosuccinimidyl 4-(N-maleimidomethyl) cyclohaxane-1-carboxylate (sulfo-SMCC) (see, e.g., Karpovsky (1984) J. Exp. Med. 160:1686; Liu (1985) Proc. Nat. Acad. Sci. USA 82:8648). Other methods include those described by Paulus (Behring Ins. Mitt. (1985) No. 78, 118-132; Brennan (1985) 5 Science 229:81-83), Glennie (1987) J Immunol. 139: 2367-2375). When the binding specificities are antibodies (e.g., two humanized antibodies), they can be conjugated via sulfhydryl bonding of the C-terminus hinge regions of the two heavy chains. In certain embodiments, the hinge region can be modified to contain an odd number of sulfhydryl residues, e.g., one, prior to conjugation.

In certain embodiments, both binding specificities of a bispecific antibody can be encoded in the same vector and expressed and assembled in the same host cell. This method is particularly useful where the bispecific and multispecific molecule is a MAb×MAb, MAb×Fab, Fab×F(ab′)₂ or ligand x Fab fusion protein. In certain embodiments, a bispecific antibody of the present disclosure can be a single chain molecule, such as a single chain bispecific antibody, a single chain bispecific molecule comprising one single chain antibody and a binding determinant or a single chain bispecific molecule comprising two binding determinants. Bispecific and multispecific molecules can also be single chain molecules or can comprise at least two single chain molecules. Methods for preparing bi- and multispecific molecules are described, for example, in U.S. Pat. Nos. 5,260,203; 5,455,030; 4,881,175; 5,132,405; 5,091,513; 5,476,786; 5,013,653; 5,258,498; and 5,482,858. Engineered antibodies with three or more functional antigen binding sites (e.g., epitope binding sites) including “Octopus antibodies,” are also included herein (see, e.g., US 2006/0025576A1).

In certain embodiments, an animal system can be used to produce an antibody of the present disclosure. One animal system for preparing hybridomas is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known (see, e.g., Harlow and Lane (1988), Antibodies, A Laboratory Manual, Cold Spring Harbor Laboratory Press, Cold Spring Harbor New York).

C. Binding Competition Assays

The anti-FGF21 antibodies of the present disclosure provided herein can be identified, screened for or characterized for their physical/chemical properties and/or biological activities by various assays known in the art and provided herein.

1. Binding Assays and Other Assays

An antibody of the present disclosure can be tested for its antigen binding activity by known methods, such enzyme-linked immunosorbent assay (ELISA), a radioimmunoassay (RIA) or a Western Blot Assay. Each of these assays generally detects the presence of protein-antibody complexes of particular interest by employing a labeled reagent (e.g., an antibody) specific for the complex of interest. For example, the FGF21-antibody complexes can be detected using, e.g., an enzyme-linked antibody or antibody fragment which recognizes and specifically binds to the antibody-FGF21 complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a Geiger counter or a scintillation counter or by autoradiography.

In certain embodiments, competition assays can be used to identify an antibody that competes with an anti-FGF21 antibody of the present disclosure, e.g., mAb4 or mAb15, for binding to FGF21. In certain embodiments, such a competing antibody binds to the same epitope (e.g., a linear or a conformational epitope) that is bound by mAb4 or mAb15. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In a non-limiting example of a competition assay, immobilized FGF21 can be incubated in a solution comprising a first labeled antibody that binds to FGF21 (e.g., mAb4 or mAb15) and a second unlabeled antibody that is being tested for its ability to compete with the first antibody for binding to FGF21. The second antibody may be present in a hybridoma supernatant. As a control, immobilized FGF21 is incubated in a solution comprising the first labeled antibody but not the second unlabeled antibody. After incubation under conditions permissive for binding of the first antibody to FGF21, excess unbound antibody is removed, and the amount of label associated with immobilized FGF21 is measured. If the amount of label associated with immobilized FGF21 is substantially reduced in the test sample relative to the control sample, then that indicates that the second antibody is competing with the first antibody for binding to FGF21. See Harlow and Lane (1988) Antibodies: A Laboratory Manual ch. 14 (Cold Spring Harbor Laboratory, Cold Spring Harbor, N.Y.).

D. Immunoconjugates

The presently disclosed subject matter further provides immunoconjugates comprising an antibody conjugated to one or more cytotoxic agents, such as chemotherapeutic agents or drugs, growth inhibitory agents, toxins (e.g., protein toxins, enzymatically active toxins of bacterial, fungal, plant or animal origin, or fragments thereof) or radioactive isotopes. For example, an antibody or antigen-binding portion of the disclosed subject matter can be functionally linked (e.g., by chemical coupling, genetic fusion, noncovalent association or otherwise) to one or more other binding molecules, such as another antibody, antibody fragment, peptide or binding mimetic.

In certain embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In certain embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In certain embodiments, an immunoconjugate comprises an antibody as described herein conjugated to a radioactive atom to form a radioconjugate. A variety of radioactive isotopes are available for the production of radioconjugates. Non-limiting examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it can include a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker can be a “cleavable linker” facilitating release of a cytotoxic drug in the cell. For example, an acid-labile linker, peptidase-sensitive linker, photolabile linker, dimethyl linker or disulfide-containing linker (Char et al., Cancer Res. 52:127-131 (1992); U.S. Pat. No. 5,208,020) can be used.

The immunuoconjugates disclosed herein expressly contemplate, but are not limited to, such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### IV. Kits

The presently disclosed subject matter further provides kits containing materials useful for performing the immunoassays disclosed herein. In certain embodiments, the kit includes a container containing an anti-FGF21 antibody disclosed herein. Non-limiting examples of suitable containers include bottles, test tubes, vials and microtiter plates. The containers can be formed from a variety of materials such as glass or plastic. In certain embodiments, the kit further includes a package insert that provides instructions for using the anti-FGF21 antibody in the disclosed immunoassay methods.

In certain embodiments, the kit can include one or more containers containing one or more anti-FGF21 antibodies. Non-limiting examples of anti-FGF21 antibodies are disclosed in Tables 8-13 and 16-19 and FIGS. 41A and B. For example, but not by way of limitation, the kit can include at least one container that includes an anti-FGF21 capture antibody and at least one container that includes an anti-FGF21 detector antibody.

In certain embodiments, a kit for detecting total FGF21 protein in a sample includes a first container containing a capture antibody that binds to an epitope present within amino acid residues 5-172 of FGF21, a second container containing a detector antibody that binds to an epitope present within amino acid residues 5-172 of FGF21 and a third container containing a detection agent.

In certain embodiments, a kit for detecting active FGF21 protein in a sample includes a first container containing a capture antibody that binds to an epitope present within amino acid residues 5-172 of FGF21, a second container containing a detector antibody that binds to an epitope present within amino acid residues 173-182 of FGF21 and a third container containing a detection agent.

In certain embodiments, a kit for determining the ratio of active FGF21 protein to total FGF21 protein in a sample includes a first container containing a first capture antibody that binds to an epitope present within amino acid residues 5-172 of FGF21, a second container containing a first detector antibody that binds to an epitope present within amino acid residues 5-172 of FGF21, a third container containing a second capture antibody that binds to an epitope present within amino acid residues 5-172 of FGF21, a fourth container containing a second detector antibody that binds to an epitope present within amino acid residues 173-182 of FGF21 and a fifth container containing a detection agent. In certain embodiments, the first and second capture antibodies are the same antibody and can be provided in a single container. Alternatively, the first and second capture antibodies are different antibodies, and can be provided in separate containers.

In certain embodiments, the capture antibody and/or the detector antibody can be provided in a kit of the present disclosure at a concentration of about 0.1 μg/ml to about 5.0 μg/ml. In certain embodiments, the detector antibody can be labeled, e.g., with biotin.

In certain embodiments, the detection agent provided in a kit of the present disclosure can be avidin, streptavidin-HRP or streptavidin-β-D-galactopyranose (SBG). In certain embodiments, a kit of the present disclosure can further include tetramethylbenzidine, hydrogen peroxide and/or resorufin β-D-galactopyranoside. In certain embodiments, if the kit includes streptavidin-HRP, then the kit can further include tetramethylbenzidine and hydrogen peroxide. In certain embodiments, if the kit includes SBG, then the kit can further include resorufin β-D-galactopyranoside. In certain embodiments, SBG can be provided in a kit at a concentration from about 100 pM to about 400 pM.

In certain embodiments, the capture antibody can be provided attached to solid support surface, such as, for example but not limited to, a plate or a bead, e.g., a paramagnetic bead. Alternatively or additionally, the kit can further include a solid support surface that can be coupled to the capture antibody. In certain embodiments, the solid support can be paramagnetic beads and can be provided at a concentration from about 0.1×10′ beads/ml to about 10.0×10′ beads/ml.

Alternatively or additionally, the kit can include other materials desirable from a commercial and user standpoint, including other buffers, diluents and filters. In certain embodiments, the kit can include materials for collecting and/or processing blood samples.

### V. Exemplary Embodiments

A. In certain non-limiting embodiments, the presently disclosed subject matter provides for an immunoassay method for determining the amount of total FGF21 protein in a sample comprising:

(a) contacting a capture antibody that binds to an epitope present within amino acid residues 5-172 of FGF21 with the sample to generate a sample-capture antibody combination material;

(b) contacting the sample-capture antibody combination material with a detector antibody that binds to an epitope present within amino acid residues 5-172 of FGF21;

(c) detecting the detector antibody bound to the sample-capture antibody combination material; and

(d) calculating an amount of total FGF21 protein present in the sample based on the level of the detector antibody bound.

A1. The foregoing immunoassay method of A, wherein the capture antibody and the detector antibody bind to different epitopes within amino acid residues 5-172 of FGF21.

B. In certain non-limiting embodiments, the presently disclosed subject matter provides for an immunoassay method for determining the amount of active FGF21 protein in a sample comprising:

(a) contacting a capture antibody that binds to an epitope present within amino acid residues 5-172 of FGF21 with the sample to generate a sample-capture antibody combination material;

(b) contacting the sample-capture antibody combination material with a detector antibody that binds to an epitope present within amino acid residues 173-182 of FGF21;

(c) detecting the detector antibody bound to the sample-capture antibody combination material; and

(d) calculating an amount of active FGF21 protein present in the sample based on the level of the detector antibody bound.

C. In certain non-limiting embodiments, the presently disclosed subject matter provides for an immunoassay method for determining the ratio of active FGF21 protein to total FGF21 protein in a sample comprising:

(a) (i) contacting a first capture antibody that binds to an epitope present within amino acid residues 5-172 of FGF21 with the sample to generate a first sample-capture antibody combination material; (ii) contacting the first sample-capture antibody combination material with a first detector antibody that binds to an epitope present within amino acid residues 5-172 of FGF21; (iii) detecting the first detector antibody bound to the sample-capture antibody combination material; and (iv) calculating an amount of total FGF21 protein present in the sample based on the level of the first detector antibody bound;

(b) (i) contacting a second capture antibody that binds to an epitope present within amino acid residues 5-172 of FGF21 with the sample to generate a second sample-capture antibody combination material; (ii) contacting the second sample-capture antibody combination material with a second detector antibody that binds to an epitope present within amino acid residues 173-182 of FGF21; (iii) detecting the second detector antibody bound to the sample-capture antibody combination material; and (iv) calculating an amount of active FGF21 protein present in the sample based on the level of the second detector antibody bound; and

(c) comparing the amount of total FGF21 protein as determined by step (a) with the amount of active FGF21 protein as determined by step (b) to determine the ratio of active FGF21 protein to total FGF21 protein in the sample.

C1. The foregoing immunoassay method of C, wherein the first capture antibody and second capture antibody are the same antibody.

C2. The foregoing immunoassay method of C, wherein the first capture antibody and the first detector antibody bind to different epitopes within amino acid residues 5-172 of FGF21.

C3. The foregoing immunoassay method of any one of A-C2, wherein the immunoassay is an enzyme-linked immunosorbent assay (ELISA).

C4. The foregoing immunoassay method of any one of A-C3, wherein one or more of the capture antibody, first capture antibody and second capture antibody is immobilized to a paramagnetic bead.

C5. The foregoing immunoassay method of any one of A-C4, wherein one or more of the detector antibody, first detector antibody and second detector antibody is conjugated to biotin.

C6. The foregoing immunoassay method of any one of A-C5, wherein one or more of the capture antibody, first capture antibody and second capture antibody binds to FGF21 with a K_{d} from about 10⁻¹⁰ M to 10⁻¹³ M.

C7. The foregoing immunoassay method of any one of A and C-C6, wherein one or more of the detector antibody and first detector antibody binds to FGF21 with a K_{d} from about 10⁻¹⁰ M to 10⁻¹³ M.

C8. The foregoing immunoassay method of any one of A-C7, wherein the sample is a blood sample.

C9. The foregoing immunoassay method of any one of A-C7, wherein the sample is a plasma sample.

C10. The foregoing immunoassay method of anyone of A-C9, wherein the method detects the amount of total or active FGF21 protein in the sample at an in-well sensitivity from about 2 pg/ml to about 20 pg/ml.

C11. The foregoing immunoassay method of anyone of A-C9, wherein the immunoassay method is performed using a single molecule detection instrument.

C12. The foregoing immunoassay method of C11, wherein the single molecule detection instrument is the Quanterix Simoa HD-1 Analyzer™.

C13. The foregoing immunoassay method of C11 and C12, wherein the method detects the amount of total or active FGF21 protein in the sample at an in-well sensitivity from about 0.2 pg/ml to about 0.5 pg/ml.

C14. The foregoing immunoassay method of anyone of A-C13, wherein one or more of the capture antibody, first capture antibody and second capture antibody comprises:

(a) a heavy chain variable region CDR1 comprising an amino acid sequence selected from the group consisting of SEQ ID NOs: 26 and 27, and conservative substitutions thereof;

(b) a heavy chain variable region CDR2 domain comprising an amino acid sequence selected from the group consisting of SEQ ID NOs: 30 and 31, and conservative substitutions thereof;

(c) a heavy chain variable region CDR3 domain comprising an amino acid sequence selected from the group consisting of SEQ ID NOs: 34 and 35, and conservative substitutions thereof;

(d) alight chain variable region CDR1 domain comprising an amino acid sequence selected from the group consisting of SEQ ID NOs: 38 and 39, and conservative substitutions thereof;

(e) a light chain variable region CDR2 domain comprising an amino acid sequence selected from the group consisting of SEQ ID NOs: 42 and 43, and conservative substitutions thereof; and

(f) a light chain variable region CDR3 domain comprising an amino acid sequence selected from the group consisting of SEQ ID NOs: 46 and 47, and conservative substitutions thereof.

C15. The foregoing immunoassay of anyone of A-C13, wherein one or more of the capture antibody, first capture antibody and second capture antibody comprises:

(a) a heavy chain variable region comprising an amino acid sequence selected from the group consisting of SEQ ID NOs: 54, 55, 74 and 75, and conservative substitutions thereof; and

(b) alight chain variable region comprising an amino acid sequence selected from the group consisting of SEQ ID NOs: 50, 51, 70 and 71, and conservative substitutions thereof.

C16. The foregoing immunoassay of anyone of A-C13, wherein one or more of the capture antibody, first capture antibody and second capture antibody comprises:

(a) a heavy chain comprising an amino acid sequence selected from the group consisting of SEQ ID NOs: 22, 23, 66 and 67, and conservative substitutions thereof; and

(b) a light chain comprising an amino acid sequence selected from the group consisting of SEQ ID NOs: 18, 19, 62 and 63, and conservative substitutions thereof.

C17. The foregoing immunoassay method of anyone of A and C-C13, wherein one or more of the detector antibody and first detector antibody comprises:

(a) a heavy chain variable region CDR1 comprising an amino acid sequence selected from the group consisting of SEQ ID NOs: 28 and 29, and conservative substitutions thereof;

(b) a heavy chain variable region CDR2 domain comprising an amino acid sequence selected from the group consisting of SEQ ID NOs: 32 and 33, and conservative substitutions thereof;

(c) a heavy chain variable region CDR3 domain comprising an amino acid sequence selected from the group consisting of SEQ ID NOs: 36 and 37, and conservative substitutions thereof;

(d) alight chain variable region CDR1 domain comprising an amino acid sequence selected from the group consisting of SEQ ID NOs: 40 and 41, and conservative substitutions thereof;

(e) a light chain variable region CDR2 domain comprising an amino acid sequence selected from the group consisting of SEQ ID NOs: 44 and 45, and conservative substitutions thereof; and

(f) a light chain variable region CDR3 domain comprising an amino acid sequence selected from the group consisting of SEQ ID NOs: 48 and 49, and conservative substitutions thereof.

C18. The foregoing immunoassay of anyone of A and C-C13, wherein one or more of the detector antibody and first detector antibody comprises:

(a) a heavy chain variable region comprising an amino acid sequence selected from the group consisting of SEQ ID NOs: 56, 57, 72 and 73, and conservative substitutions thereof; and

(b) alight chain variable region comprising an amino acid sequence selected from the group consisting of SEQ ID NOs: 52, 53, 68 and 69, and conservative substitutions thereof.

C19. The foregoing immunoassay of anyone of A and C-C13, wherein one or more of the detector antibody and first detector antibody comprises:

(a) a heavy chain comprising an amino acid sequence selected from the group consisting of SEQ ID NOs: 24, 25, 64 and 65, and conservative substitutions thereof; and

(b) a light chain comprising an amino acid sequence selected from the group consisting of SEQ ID NOs: 20, 21, 60 and 61, and conservative substitutions thereof.

C20. The foregoing immunoassay method of C14, wherein one or more of the capture antibody, first capture antibody and second capture antibody comprises:

(a) a heavy chain variable region CDR1 comprising the amino acid sequence of SEQ ID NO: 26, and conservative substitutions thereof;

(b) a heavy chain variable region CDR2 domain comprising the amino acid sequence of SEQ ID NO: 30, and conservative substitutions thereof;

(c) a heavy chain variable region CDR3 domain comprising the amino acid sequence of SEQ ID NO: 34, and conservative substitutions thereof;

(d) a light chain variable region CDR1 domain comprising the amino acid sequence of SEQ ID NO: 38, and conservative substitutions thereof;

(e) a light chain variable region CDR2 domain comprising the amino acid sequence of SEQ ID NO: 42, and conservative substitutions thereof; and

(f) a light chain variable region CDR3 domain comprising the amino acid sequence of SEQ ID NO: 46, and conservative substitutions thereof.

C21. The foregoing immunoassay of C20, wherein one or more of the capture antibody, first capture antibody and second capture antibody comprises:

(a) a heavy chain variable region comprising the amino acid sequence of SEQ ID NO: 54, and conservative substitutions thereof; and

(b) a light chain variable region comprising the amino acid sequence of SEQ ID NO: 50, and conservative substitutions thereof.

C22. The foregoing immunoassay of C21, wherein one or more of the capture antibody, first capture antibody and second capture antibody comprises:

(a) a heavy chain comprising the amino acid sequence of SEQ ID NO: 22, and conservative substitutions thereof; and

(b) a light chain comprising the amino acid sequence of SEQ ID NO: 18, and conservative substitutions thereof.

C23. The foregoing immunoassay method of C17, wherein one or more of the detector antibody and first detector antibody comprises:

(a) a heavy chain variable region CDR1 comprising the amino acid sequence of SEQ ID NO: 29, and conservative substitutions thereof;

(b) a heavy chain variable region CDR2 domain comprising the amino acid sequence of SEQ ID NO: 33, and conservative substitutions thereof;

(c) a heavy chain variable region CDR3 domain comprising the amino acid sequence of SEQ ID NO: 37, and conservative substitutions thereof;

(d) a light chain variable region CDR1 domain comprising the amino acid sequence of SEQ ID NO: 41, and conservative substitutions thereof;

(e) a light chain variable region CDR2 domain comprising the amino acid sequence of SEQ ID NO: 45, and conservative substitutions thereof; and

(f) a light chain variable region CDR3 domain comprising the amino acid sequence of SEQ ID NO: 49, and conservative substitutions thereof.

C24. The foregoing immunoassay of C23, wherein one or more of the detector antibody and first detector antibody comprises:

(a) a heavy chain variable region comprising the amino acid sequence of SEQ ID NO: 57, and conservative substitutions thereof; and

(b) a light chain variable region comprising the amino acid sequence of SEQ ID NO: 53, and conservative substitutions thereof.

C25. The foregoing immunoassay of C24, wherein one or more of the detector antibody and first detector antibody comprises:

(a) a heavy chain comprising the amino acid sequence of SEQ ID NO: 25, and conservative substitutions thereof; and

(b) a light chain comprising the amino acid sequence of SEQ ID NO: 21, and conservative substitutions thereof.

C26. The foregoing immunoassay method of anyone of A-C13, wherein one or more of the capture antibody, first capture antibody and second capture antibody competitively binds with an antibody comprising:

(a) a heavy chain variable region CDR1 comprising an amino acid sequence selected from the group consisting of SEQ ID NOs: 26 and 27, and conservative substitutions thereof;

(b) a heavy chain variable region CDR2 domain comprising an amino acid sequence selected from the group consisting of SEQ ID NOs: 30 and 31, and conservative substitutions thereof;

(c) a heavy chain variable region CDR3 domain comprising an amino acid sequence selected from the group consisting of SEQ ID NOs: 34 and 35, and conservative substitutions thereof;

(d) alight chain variable region CDR1 domain comprising an amino acid sequence selected from the group consisting of SEQ ID NOs: 38 and 39, and conservative substitutions thereof;

(e) a light chain variable region CDR2 domain comprising an amino acid sequence selected from the group consisting of SEQ ID NOs: 42 and 43, and conservative substitutions thereof; and

(f) a light chain variable region CDR3 domain comprising an amino acid sequence selected from the group consisting of SEQ ID NOs: 46 and 47, and conservative substitutions thereof.

C27. The foregoing immunoassay method of anyone of A and C-C13, wherein one or more of the detector antibody and first detector antibody competitively binds with an antibody comprising:

(a) a heavy chain variable region CDR1 comprising an amino acid sequence selected from the group consisting of SEQ ID NOs: 28 and 29, and conservative substitutions thereof;

(b) a heavy chain variable region CDR2 domain comprising an amino acid sequence selected from the group consisting of SEQ ID NOs: 32 and 33, and conservative substitutions thereof;

(c) a heavy chain variable region CDR3 domain comprising an amino acid sequence selected from the group consisting of SEQ ID NOs: 36 and 37, and conservative substitutions thereof;

(d) alight chain variable region CDR1 domain comprising an amino acid sequence selected from the group consisting of SEQ ID NOs: 40 and 41, and conservative substitutions thereof;

(e) a light chain variable region CDR2 domain comprising an amino acid sequence selected from the group consisting of SEQ ID NOs: 44 and 45, and conservative substitutions thereof; and

(f) a light chain variable region CDR3 domain comprising an amino acid sequence selected from the group consisting of SEQ ID NOs: 48 and 49, and conservative substitutions thereof.

D. In certain non-limiting embodiments, the presently disclosed subject matter provides for a kit for detecting total FGF21 protein in a sample comprising:

(a) a capture antibody that binds to an epitope present within amino acid residues 5-172 of FGF21;

(b) a detector antibody that binds to an epitope present within amino acid residues 5-172 of FGF21; and

(c) a detection agent.

D1. The foregoing kit of D, wherein the capture antibody and the detector antibody bind to different epitopes within amino acid residues 5-172 of FGF21.

E. In certain non-limiting embodiments, the presently disclosed subject matter provides for a kit for detecting active FGF21 protein in a sample comprising:

(a) a capture antibody that binds to an epitope present within amino acid residues 5-172 of FGF21;

(b) a detector antibody that binds to an epitope present within amino acid residues 173-182 of FGF21; and

(c) a detection agent.

F. In certain non-limiting embodiments, the presently disclosed subject matter provides for a kit for determining the ratio of active FGF21 protein to total FGF21 protein in a sample comprising:

(a) (i) a first capture antibody that binds to an epitope present within amino acid residues 5-172 of FGF21 and (ii) a first detector antibody that binds to an epitope present within amino acid residues 5-172 of FGF21;

(b) (i) a second capture antibody that binds to an epitope present within amino acid residues 5-172 of FGF21 and (ii) a second detector antibody that binds to an epitope present within amino acid residues 173-182 of FGF21; and

(c) one or more detection agents.

F1. The foregoing kit of F, wherein the first capture antibody and second capture antibody are the same antibody.

F2. The foregoing kit of F, wherein the first capture antibody and the first detector antibody bind to different epitopes within amino acid residues 5-172 of FGF21.

F3. The foregoing kit of any one of D-F2, wherein one or more of the capture antibody, first capture antibody and second capture antibody is immobilized to a paramagnetic bead.

F4. The foregoing kit of any one of D-F3, wherein one or more of the detector antibody, first detector antibody and second detector antibody is conjugated to biotin.

F5. The foregoing kit of any one of D-F4, wherein the detection agent is selected from the group consisting of a streptavidin-β-D-galactopyranose conjugate, a streptavidin-horseradish peroxidase conjugate and a combination thereof.

F6. The foregoing kit of F5 further comprising resorufin β-D-galactopyranoside, tetramethylbenzidine, hydrogen peroxide or combinations thereof.

F7. The foregoing kit of any one of D-F6, wherein one or more of the capture antibody, first capture antibody and second capture antibody binds to FGF21 with a K_{d} from about 10⁻¹⁰ M to 10⁻¹³ M.

F8. The foregoing kit of any one of D and F-F7, wherein one or more of the detector antibody and first detector antibody binds to FGF21 with a K_{d} from about 10⁻¹⁰ M to 10⁻¹³ M.

F9. The foregoing kit of any one of D and F-F8, wherein the detector antibody or first detector antibody has a concentration from about 0.1 μg/ml to about 1 μg/ml.

F10. The foregoing kit of any one of E-F7, wherein one or more of the detector antibody or second detector antibody has a concentration from about 1 μg/ml to about 3 μg/ml.

F11. The foregoing kit of F5, wherein the streptavidin-β-D-galactopyranose conjugate has a concentration from about 100 pM to about 400 pM.

F12. The foregoing kit of anyone of D-F11, wherein one or more of the capture antibody, first capture antibody and second capture antibody comprises:

(a) a heavy chain variable region CDR1 comprising an amino acid sequence selected from the group consisting of SEQ ID NOs: 26 and 27, and conservative substitutions thereof;

(b) a heavy chain variable region CDR2 domain comprising an amino acid sequence selected from the group consisting of SEQ ID NOs: 30 and 31, and conservative substitutions thereof;

(c) a heavy chain variable region CDR3 domain comprising an amino acid sequence selected from the group consisting of SEQ ID NOs: 34 and 35, and conservative substitutions thereof;

(d) alight chain variable region CDR1 domain comprising an amino acid sequence selected from the group consisting of SEQ ID NOs: 38 and 39, and conservative substitutions thereof;

(e) a light chain variable region CDR2 domain comprising an amino acid sequence selected from the group consisting of SEQ ID NOs: 42 and 43, and conservative substitutions thereof; and

(f) a light chain variable region CDR3 domain comprising an amino acid sequence selected from the group consisting of SEQ ID NOs: 46 and 47, and conservative substitutions thereof.

F13. The foregoing kit of anyone of D-F11, wherein one or more of the capture antibody, first capture antibody and second capture antibody comprises:

(a) a heavy chain variable region comprising an amino acid sequence selected from the group consisting of SEQ ID NOs: 54, 55, 74 and 75, and conservative substitutions thereof; and

(b) alight chain variable region comprising an amino acid sequence selected from the group consisting of SEQ ID NOs: 50, 51, 70 and 71, and conservative substitutions thereof.

F14. The foregoing kit of anyone of D-F11, wherein one or more of the capture antibody, first capture antibody and second capture antibody comprises:

(a) a heavy chain comprising an amino acid sequence selected from the group consisting of SEQ ID NOs: 22, 23, 66 and 67, and conservative substitutions thereof; and

(b) a light chain comprising an amino acid sequence selected from the group consisting of SEQ ID NOs: 18, 19, 62 and 63, and conservative substitutions thereof.

F15. The foregoing kit of anyone of D and F-F11, wherein one or more of the detector antibody and first detector antibody comprises:

(a) a heavy chain variable region CDR1 comprising an amino acid sequence selected from the group consisting of SEQ ID NOs: 28 and 29, and conservative substitutions thereof;

(b) a heavy chain variable region CDR2 domain comprising an amino acid sequence selected from the group consisting of SEQ ID NOs: 32 and 33, and conservative substitutions thereof;

(c) a heavy chain variable region CDR3 domain comprising an amino acid sequence selected from the group consisting of SEQ ID NOs: 36 and 37, and conservative substitutions thereof;

(d) alight chain variable region CDR1 domain comprising an amino acid sequence selected from the group consisting of SEQ ID NOs: 40 and 41, and conservative substitutions thereof;

(e) a light chain variable region CDR2 domain comprising an amino acid sequence selected from the group consisting of SEQ ID NOs: 44 and 45, and conservative substitutions thereof; and

(f) a light chain variable region CDR3 domain comprising an amino acid sequence selected from the group consisting of SEQ ID NOs: 48 and 49, and conservative substitutions thereof.

F16. The foregoing kit of anyone of D and F-F11, wherein one or more of the detector antibody and first detector antibody comprises:

(a) a heavy chain variable region comprising an amino acid sequence selected from the group consisting of SEQ ID NOs: 56, 57, 72 and 73, and conservative substitutions thereof; and

(b) alight chain variable region comprising an amino acid sequence selected from the group consisting of SEQ ID NOs: 52, 53, 68 and 69, and conservative substitutions thereof.

F17. The foregoing kit of anyone of D and F-F11, wherein one or more of the detector antibody and first detector antibody comprises:

(a) a heavy chain comprising an amino acid sequence selected from the group consisting of SEQ ID NOs: 24, 25, 64 and 65, and conservative substitutions thereof; and

(b) a light chain comprising an amino acid sequence selected from the group consisting of SEQ ID NOs: 20, 21, 60 and 61, and conservative substitutions thereof.

F18. The foregoing kit of F12, wherein one or more of the capture antibody, first capture antibody and second capture antibody comprises:

(a) a heavy chain variable region CDR1 comprising the amino acid sequence of SEQ ID NO: 26, and conservative substitutions thereof;

(b) a heavy chain variable region CDR2 domain comprising the amino acid sequence of SEQ ID NO: 30, and conservative substitutions thereof;

(c) a heavy chain variable region CDR3 domain comprising the amino acid sequence of SEQ ID NO: 34, and conservative substitutions thereof;

(d) a light chain variable region CDR1 domain comprising the amino acid sequence of SEQ ID NO: 38, and conservative substitutions thereof;

(e) a light chain variable region CDR2 domain comprising the amino acid sequence of SEQ ID NO: 42, and conservative substitutions thereof; and

(f) a light chain variable region CDR3 domain comprising the amino acid sequence of SEQ ID NO: 46, and conservative substitutions thereof.

F19. The foregoing kit of F18, wherein one or more of the capture antibody, first capture antibody and second capture antibody comprises:

(a) a heavy chain variable region comprising the amino acid sequence of SEQ ID NO: 54, and conservative substitutions thereof; and

(b) a light chain variable region comprising the amino acid sequence of SEQ ID NO: 50, and conservative substitutions thereof.

F20. The foregoing kit of F19, wherein one or more of the capture antibody, first capture antibody and second capture antibody comprises:

(a) a heavy chain comprising the amino acid sequence of SEQ ID NO: 22, and conservative substitutions thereof; and

(b) a light chain comprising the amino acid sequence of SEQ ID NO: 18, and conservative substitutions thereof.

F21. The foregoing kit of F15, wherein one or more of the detector antibody and first detector antibody comprises:

(a) a heavy chain variable region CDR1 comprising the amino acid sequence of SEQ ID NO: 29, and conservative substitutions thereof;

(b) a heavy chain variable region CDR2 domain comprising the amino acid sequence of SEQ ID NO: 33, and conservative substitutions thereof;

(c) a heavy chain variable region CDR3 domain comprising the amino acid sequence of SEQ ID NO: 37, and conservative substitutions thereof;

(d) a light chain variable region CDR1 domain comprising the amino acid sequence of SEQ ID NO: 41, and conservative substitutions thereof;

(e) a light chain variable region CDR2 domain comprising the amino acid sequence of SEQ ID NO: 45, and conservative substitutions thereof; and

(f) a light chain variable region CDR3 domain comprising the amino acid sequence of SEQ ID NO: 49, and conservative substitutions thereof.

F22. The foregoing kit of F21, wherein one or more of the detector antibody and first detector antibody comprises:

(a) a heavy chain variable region comprising the amino acid sequence of SEQ ID NO: 57, and conservative substitutions thereof; and

(b) a light chain variable region comprising the amino acid sequence of SEQ ID NO: 53, and conservative substitutions thereof.

F23. The foregoing kit of F22, wherein one or more of the detector antibody and first detector antibody comprises:

(a) a heavy chain comprising the amino acid sequence of SEQ ID NO: 25, and conservative substitutions thereof; and

(b) a light chain comprising the amino acid sequence of SEQ ID NO: 21, and conservative substitutions thereof.

F24. The foregoing kit of anyone of D-F11, wherein one or more of the capture antibody, first capture antibody and second capture antibody competitively binds with an antibody comprising:

(a) a heavy chain variable region CDR1 comprising an amino acid sequence selected from the group consisting of SEQ ID NOs: 26 and 27, and conservative substitutions thereof;

(b) a heavy chain variable region CDR2 domain comprising an amino acid sequence selected from the group consisting of SEQ ID NOs: 30 and 31, and conservative substitutions thereof;

(c) a heavy chain variable region CDR3 domain comprising an amino acid sequence selected from the group consisting of SEQ ID NOs: 34 and 35, and conservative substitutions thereof;

(d) alight chain variable region CDR1 domain comprising an amino acid sequence selected from the group consisting of SEQ ID NOs: 38 and 39, and conservative substitutions thereof;

(e) a light chain variable region CDR2 domain comprising an amino acid sequence selected from the group consisting of SEQ ID NOs: 42 and 43, and conservative substitutions thereof; and

(f) a light chain variable region CDR3 domain comprising an amino acid sequence selected from the group consisting of SEQ ID NOs: 46 and 47, and conservative substitutions thereof.

F25. The foregoing kit of any one of D and F-F11, wherein one or more of the detector antibody and first detector antibody competitively binds with an antibody comprising:

(a) a heavy chain variable region CDR1 comprising an amino acid sequence selected from the group consisting of SEQ ID NOs: 28 and 29, and conservative substitutions thereof;

(b) a heavy chain variable region CDR2 domain comprising an amino acid sequence selected from the group consisting of SEQ ID NOs: 32 and 33, and conservative substitutions thereof;

(c) a heavy chain variable region CDR3 domain comprising an amino acid sequence selected from the group consisting of SEQ ID NOs: 36 and 37, and conservative substitutions thereof;

(d) alight chain variable region CDR1 domain comprising an amino acid sequence selected from the group consisting of SEQ ID NOs: 40 and 41, and conservative substitutions thereof;

(e) a light chain variable region CDR2 domain comprising an amino acid sequence selected from the group consisting of SEQ ID NOs: 44 and 45, and conservative substitutions thereof; and

(f) a light chain variable region CDR3 domain comprising an amino acid sequence selected from the group consisting of SEQ ID NOs: 48 and 49, and conservative substitutions thereof.

F26. The foregoing kit of any one of D-F25, wherein the sample is a blood sample.

F27. The foregoing kit of any one of D-F25, wherein the sample is a plasma sample.

F28. The foregoing kit of any one of D-F27, wherein the kit detects the amount of total or active FGF21 protein in the sample at an in-well sensitivity from about 0.2 pg/ml to about 0.5 pg/ml.

G. In certain non-limiting embodiments, the presently disclosed subject matter provides for an isolated anti-FGF21 antibody, or an antigen-binding portion thereof, comprising:

(a) a heavy chain variable region CDR1 comprising an amino acid sequence selected from the group consisting of SEQ ID NOs: 26-29, and conservative substitutions thereof;

(b) a heavy chain variable region CDR2 domain comprising an amino acid sequence selected from the group consisting of SEQ ID NOs: 30-33, and conservative substitutions thereof;

(c) a heavy chain variable region CDR3 domain comprising an amino acid sequence selected from the group consisting of SEQ ID NOs: 34-37, and conservative substitutions thereof;

(d) alight chain variable region CDR1 domain comprising an amino acid sequence selected from the group consisting of SEQ ID NOs: 38-41, and conservative substitutions thereof;

(e) a light chain variable region CDR2 domain comprising an amino acid sequence selected from the group consisting of SEQ ID NOs: 42-45, and conservative substitutions thereof; and

(f) a light chain variable region CDR3 domain comprising an amino acid sequence selected from the group consisting of SEQ ID NOs: 46-49, and conservative substitutions thereof.

G1. The foregoing isolated antibody of G, wherein the antibody, or antigen-binding portion thereof, comprises:

(a) a heavy chain variable region CDR1 comprising the amino acid sequence of SEQ ID NO: 26, and conservative substitutions thereof;

(b) a heavy chain variable region CDR2 domain comprising the amino acid sequence of SEQ ID NO: 30, and conservative substitutions thereof;

(c) a heavy chain variable region CDR3 domain comprising the amino acid sequence of SEQ ID NO: 34, and conservative substitutions thereof;

(d) a light chain variable region CDR1 domain comprising the amino acid sequence of SEQ ID NO: 38, and conservative substitutions thereof;

(e) a light chain variable region CDR2 domain comprising the amino acid sequence of SEQ ID NO: 42, and conservative substitutions thereof; and

(f) a light chain variable region CDR3 domain comprising the amino acid sequence of SEQ ID NO: 46, and conservative substitutions thereof.

G2. The foregoing isolated antibody of G, wherein the antibody, or antigen-binding portion thereof, comprises:

(a) a heavy chain variable region CDR1 comprising the amino acid sequence of SEQ ID NO: 27, and conservative substitutions thereof;

(b) a heavy chain variable region CDR2 domain comprising the amino acid sequence of SEQ ID NO: 31, and conservative substitutions thereof;

(c) a heavy chain variable region CDR3 domain comprising the amino acid sequence of SEQ ID NO: 35, and conservative substitutions thereof;

(d) a light chain variable region CDR1 domain comprising the amino acid sequence of SEQ ID NO: 39, and conservative substitutions thereof;

(e) a light chain variable region CDR2 domain comprising the amino acid sequence of SEQ ID NO: 43, and conservative substitutions thereof; and

(f) a light chain variable region CDR3 domain comprising the amino acid sequence of SEQ ID NO: 47, and conservative substitutions thereof.

G3. The foregoing isolated antibody of G, wherein the antibody, or antigen-binding portion thereof, comprises:

(a) a heavy chain variable region CDR1 comprising the amino acid sequence of SEQ ID NO: 28, and conservative substitutions thereof;

(b) a heavy chain variable region CDR2 domain comprising the amino acid sequence of SEQ ID NO: 32, and conservative substitutions thereof;

(c) a heavy chain variable region CDR3 domain comprising the amino acid sequence of SEQ ID NO: 36, and conservative substitutions thereof;

(d) a light chain variable region CDR1 domain comprising the amino acid sequence of SEQ ID NO: 40, and conservative substitutions thereof;

(e) a light chain variable region CDR2 domain comprising the amino acid sequence of SEQ ID NO: 44, and conservative substitutions thereof; and

(f) a light chain variable region CDR3 domain comprising the amino acid sequence of SEQ ID NO: 48, and conservative substitutions thereof.

G4. The foregoing isolated antibody of G, wherein the antibody, or antigen-binding portion thereof, comprises:

(a) a heavy chain variable region CDR1 comprising the amino acid sequence of SEQ ID NO: 29, and conservative substitutions thereof;

(b) a heavy chain variable region CDR2 domain comprising the amino acid sequence of SEQ ID NO: 33, and conservative substitutions thereof;

(c) a heavy chain variable region CDR3 domain comprising the amino acid sequence of SEQ ID NO: 37, and conservative substitutions thereof;

(d) a light chain variable region CDR1 domain comprising the amino acid sequence of SEQ ID NO: 41, and conservative substitutions thereof;

(e) a light chain variable region CDR2 domain comprising the amino acid sequence of SEQ ID NO: 45, and conservative substitutions thereof; and

(f) a light chain variable region CDR3 domain comprising the amino acid sequence of SEQ ID NO: 49, and conservative substitutions thereof.

G5. The foregoing isolated antibody of G1, wherein the antibody, or antigen-binding portion thereof, comprises:

(a) a heavy chain variable region comprising the amino acid sequence of SEQ ID NO: 54, and conservative substitutions thereof; and

(b) a light chain variable region comprising the amino acid sequence of SEQ ID NO: 50, and conservative substitutions thereof.

G6. The foregoing isolated antibody of G2, wherein the antibody, or antigen-binding portion thereof, comprises:

(a) a heavy chain variable region comprising the amino acid sequence of SEQ ID NO: 55, and conservative substitutions thereof; and

(b) a light chain variable region comprising the amino acid sequence of SEQ ID NO: 51, and conservative substitutions thereof.

G7. The foregoing isolated antibody of G3, wherein the antibody, or antigen-binding portion thereof, comprises:

(a) a heavy chain variable region comprising the amino acid sequence of SEQ ID NO: 56, and conservative substitutions thereof; and

(b) a light chain variable region comprising the amino acid sequence of SEQ ID NO: 52, and conservative substitutions thereof.

G8. The foregoing isolated antibody of G4, wherein the antibody, or antigen-binding portion thereof, comprises:

(a) a heavy chain variable region comprising the amino acid sequence of SEQ ID NO: 57, and conservative substitutions thereof; and

(b) a light chain variable region comprising the amino acid sequence of SEQ ID NO: 53, and conservative substitutions thereof.

G9. The foregoing isolated antibody of G1, wherein the antibody, or antigen-binding portion thereof, comprises:

(a) a heavy chain variable region comprising the amino acid sequence of SEQ ID NO: 75, and conservative substitutions thereof; and

(b) a light chain variable region comprising the amino acid sequence of SEQ ID NO: 71, and conservative substitutions thereof.

G10. The foregoing isolated antibody of G2, wherein the antibody, or antigen-binding portion thereof, comprises:

(a) a heavy chain variable region comprising the amino acid sequence of SEQ ID NO: 74, and conservative substitutions thereof; and

(b) a light chain variable region comprising the amino acid sequence of SEQ ID NO: 70, and conservative substitutions thereof.

G11. The foregoing isolated antibody of G3, wherein the antibody, or antigen-binding portion thereof, comprises:

(a) a heavy chain variable region comprising the amino acid sequence of SEQ ID NO: 73, and conservative substitutions thereof; and

(b) a light chain variable region comprising the amino acid sequence of SEQ ID NO: 69, and conservative substitutions thereof.

G12. The foregoing isolated antibody of G4, wherein the antibody, or antigen-binding portion thereof, comprises:

(a) a heavy chain variable region comprising the amino acid sequence of SEQ ID NO: 72, and conservative substitutions thereof; and

(b) a light chain variable region comprising the amino acid sequence of SEQ ID NO: 68, and conservative substitutions thereof.

G13. The foregoing isolated antibody of G5, wherein the antibody, or antigen-binding portion thereof, comprises:

(a) a heavy chain comprising the amino acid sequence of SEQ ID NO: 22, and conservative substitutions thereof; and

(b) a light chain comprising the amino acid sequence of SEQ ID NO: 18, and conservative substitutions thereof.

G14. The foregoing isolated antibody of G6, wherein the antibody, or antigen-binding portion thereof, comprises:

(a) a heavy chain comprising the amino acid sequence of SEQ ID NO: 23, and conservative substitutions thereof; and

(b) a light chain comprising the amino acid sequence of SEQ ID NO: 19, and conservative substitutions thereof.

G15. The foregoing isolated antibody of G7, wherein the antibody, or antigen-binding portion thereof, comprises:

(a) a heavy chain comprising the amino acid sequence of SEQ ID NO: 24, and conservative substitutions thereof; and

(b) a light chain comprising the amino acid sequence of SEQ ID NO: 20, and conservative substitutions thereof.

G16. The foregoing isolated antibody of G8, wherein the antibody, or antigen-binding portion thereof, comprises:

(a) a heavy chain comprising the amino acid sequence of SEQ ID NO: 25, and conservative substitutions thereof; and

(b) a light chain comprising the amino acid sequence of SEQ ID NO: 21, and conservative substitutions thereof.

G17. The foregoing isolated antibody of G9, wherein the antibody, or antigen-binding portion thereof, comprises:

(a) a heavy chain comprising the amino acid sequence of SEQ ID NO: 67, and conservative substitutions thereof; and

(b) a light chain comprising the amino acid sequence of SEQ ID NO: 63, and conservative substitutions thereof.

G18. The foregoing isolated antibody of G10, wherein the antibody, or antigen-binding portion thereof, comprises:

(a) a heavy chain comprising the amino acid sequence of SEQ ID NO: 66, and conservative substitutions thereof; and

(b) a light chain comprising the amino acid sequence of SEQ ID NO: 62, and conservative substitutions thereof.

G19. The foregoing isolated antibody of G11, wherein the antibody, or antigen-binding portion thereof, comprises:

(a) a heavy chain comprising the amino acid sequence of SEQ ID NO: 65, and conservative substitutions thereof; and

(b) a light chain comprising the amino acid sequence of SEQ ID NO: 61, and conservative substitutions thereof.

G20. The foregoing isolated antibody of G12, wherein the antibody, or antigen-binding portion thereof, comprises:

(a) a heavy chain comprising the amino acid sequence of SEQ ID NO: 64, and conservative substitutions thereof; and

(b) a light chain comprising the amino acid sequence of SEQ ID NO: 60, and conservative substitutions thereof.

H. In certain non-limiting embodiments, the presently disclosed subject matter provides for an isolated nucleic acid encoding the antibody, or antigen-binding portion thereof, of any one of G-G20.

I. In certain non-limiting embodiments, the presently disclosed subject matter provides for a host cell comprising the nucleic acid of H.

J. In certain non-limiting embodiments, the presently disclosed subject matter provides for a method of producing an antibody comprising culturing the host cell of I so that the antibody is produced.

J1. The foregoing method of J, further comprising recovering the antibody from the host cell.

K. In certain non-limiting embodiments, the presently disclosed subject matter provides for a composition comprising one or more antibodies, or antigen-binding portions thereof, of any one of G-G20.

### EXAMPLES

The following examples are merely illustrative of the presently disclosed subject matter and should not be considered as limitations in any way.

### Example 1: Anti-FGF21 Antibody Generation

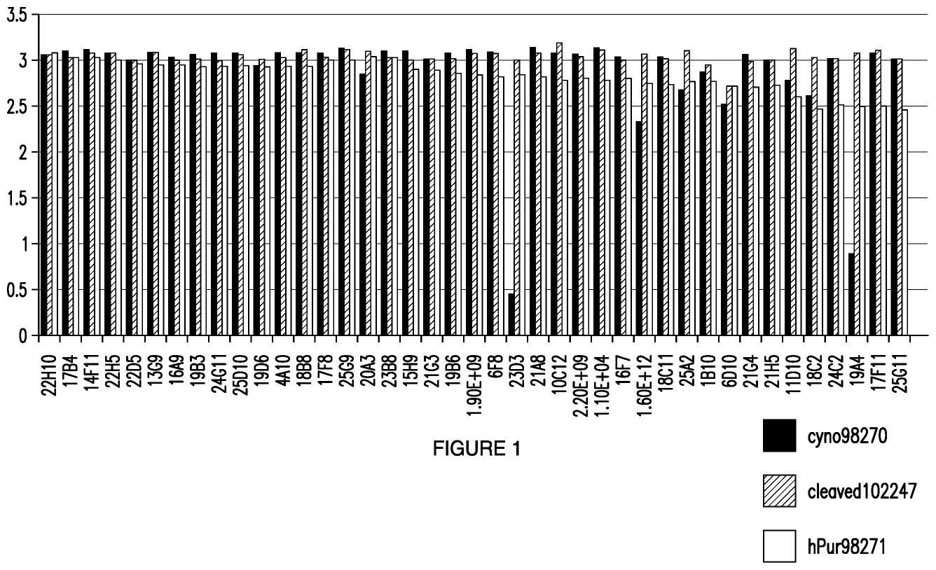

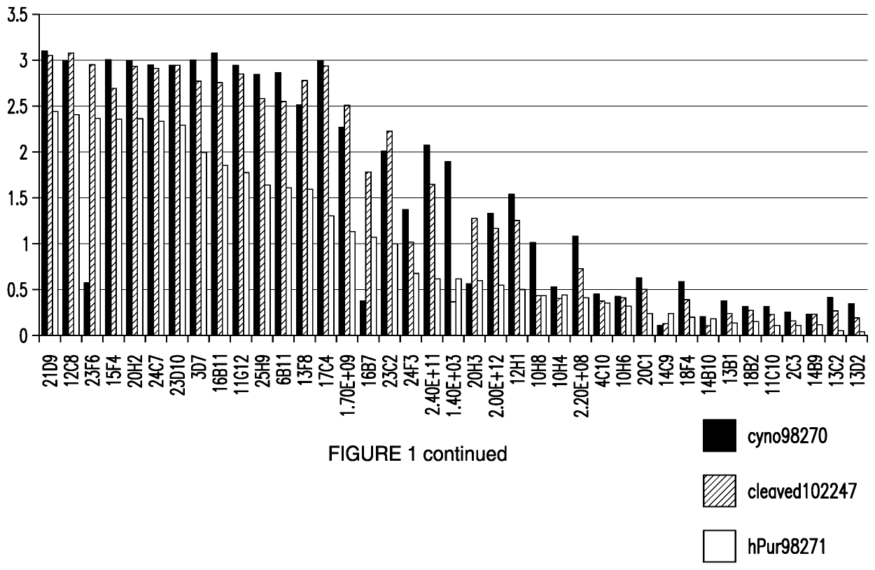

Monoclonal antibodies were generated by immunizing SJL and Balb/c mice with recombinant human FGF21. 80 hybridoma supernatants were screened by ELISA (FIG. 1). 20 hybridomas were selected based on binding to intact human FGF21 (PUR 98271), intact cynomolgus FGF21 (PUR 98270) and cleaved human FGF21 (PUR 102247) generated by digesting intact human FGF21 by human FAP.

### Example 2: Anti-FGF21 Antibody Characterization

IgG obtained from the selected 20 hybridomas identified in Example 1 were further characterized by ELISA. ELISA was performed as follows: 96 well MaxiSorp plate (439454, Nalge Nunc International; Rochester, N.Y.) was coated with 1 μg/mL of anti-FGF21 mAbs or anti-FGF21 sheep pAbs (Cat. No. RD184108100, Biovendor, Asheville, N.C.) in coating buffer (50 mM sodium carbonate, pH 9.6) overnight at 4° C. On the next day, after blocking with PBS containing 0.5% BSA and 10 ppm ProClin pH 7.4, and washing with the Washing buffer (PBS, 0.05% Tween 20, pH7.2), plate was incubated with 0.00000186-2000 pg/mL of intact human FGF21 (full-length, uncleaved FGF21; Cat. No. 2539-FG, R&D Systems) or the FAP-cleaved human FGF21 in assay buffer (25 mM HEPES, pH 7.2, 150 mM NaCl, 0.2 mM CaCl₂, 0.1% bovine serum albumin (BSA), 0.05% Tween 20) for 1-2 h at room temperature. After washing with the Washing buffer, the plate was incubated with 0.5 μg/ml of the secondary antibody (R&D Systems, biotinylated goat anti-FGF21 pAb BAF2539) for 1-2 hr at room temperature. After washing with Washing buffer, the plate was incubated with High Sensitivity Streptavidin-HRP (PIERCE Cat. No. 21130) diluted 1:1,000 in assay buffer. After washing with the Washing buffer, the binding of anti-FGF21 to recombinant FGF21 was assessed by adding substrate 3, 3′, 5, 5′ tetramethyl benzidine (TMBE 1000, Moss; Pasadena, Md.). The mean absorbance values from duplicate wells were plotted as a function of antibody concentration and the data were fitted to a three parameter equation to calculate the half maximal effective concentration (EC₅₀) values for each antibody using Prism 6 (GraphPad Software, Inc., La Jolla, Calif.) (Table 2).

TABLE 2
EC₅₀ values for each FGF21 antibody.

| | | Intact | Cleaved | EC₅₀ |
|---|---|---|---|---|
| | | FGF21 | FGF21 | ratio |
| Primary | | EC₅₀ | EC₅₀ | (Cleaved/ |
| Ab | Secondary Ab | (pg/ml) | (pg/ml) | Intact) |
| mAbl | Goat anti-FGF21 pAb | 126 | 228 | 1.8 |
| mAb2 | Goat anti-FGF21 pAb | 2108 | 3187 | 1.5 |
| mAb3 | Goat anti-FGF21 pAb | 292 | 450 | 1.5 |
| mAb4 | Goat anti-FGF21 pAb | 90 | 170 | 1.9 |
| mAb5 | Goat anti-FGF21 pAb | 5506 | 19331 | 3.5 |
| mAb6 | Goat anti-FGF21 pAb | 1993 | 4813 | 2.4 |
| mAb7 | Goat anti-FGF21 pAb | 8797 | 25403 | 2.9 |
| mAb8 | Goat anti-FGF21 pAb | 503 | 777 | 1.5 |
| mAb9 | Goat anti-FGF21 pAb | 855 | 1385 | 1.6 |
| mAb10 | Goat anti-FGF21 pAb | 136 | 249 | 1.8 |
| mAb11 | Goat anti-FGF21 pAb | 149 | 318 | 2.1 |
| mAb12 | Goat anti-FGF21 pAb | 5633 | 30386 | 5.4 |
| mAb13 | Goat anti-FGF21 pAb | 169 | 300 | 1.8 |
| Sheep | Goat anti-FGF21 pAb | 48 | 84 | 1.7 |
| pAb | | | | |

On the basis of differential detection of intact versus cleaved FGF21 and absolute EC₅₀ values, antibodies mAb5, mAb6, mAb7 and mAb12 were excluded from further analysis. Antibodies mAb1, mAb2, mAb3, mAb4, mAb8, mAb9, mAb10, mAb11, mAb13, mAb15 and mAb16 were biotinylated by using EZ-Link™ NHS-PEG Solid-Phase Biotinylation Kit (PIERCE Cat. No. 21450) and sandwich ELISA was conducted in a pairwise combinatorial manner using intact FGF21 (Tables 3 and 4). Biotinylated goat anti-FGF21 pAb BAF2539 (R&D Systems) was used as a positive control.

TABLE 3
Compatibility of anti-FGF21 mAbs in sandwich ELISA.

| | BIO-1 | BIO-10 | BIO-11 | BIO-4 | BIO-9 | BIO-13 | BIO-2 | BIO-3 | BIO-8 | BAF2539 |
|---|---|---|---|---|---|---|---|---|---|---|
| mAb1 | | | | XX | XX | | | | | XX |
| mAb10 | | | | XX | XX | X | | | | XX |
| mAb11 | | | | XX | XX | X | | | | XX |
| mAb4 | XX | XX | XX | | | | | | | XX |
| mAb9 | XX | XX | XX | | | | | | | XX |
| mAb13 | X | X | X | | | | | | | XX |
| mAb8 | XX | XX | XX | XX | | X | | X | | XX |
| mAb2 | | | | | | | | | | XX |
| mAb3 | | | | | | | | | | XX |
| Sheep | | | | | | | | | | XX |
| pAb | | | | | | | | | | |

XX: strong signal with OD > 1,
X: strong signal with OD < 1

TABLE 4
Compatibility of anti-FGF21 mAbs in sandwich ELISA.

| | BIO-4 | BIO-11 | BIO-15 | BIO-16 |
|---|---|---|---|---|
| mAb4 | | XX | XX | X |
| mAb8 | — | — | — | |
| mAb9 | | XX | XX | X |
| mAb11 | XX | | XX | |
| mAb15 | XX | X | | |
| mAb16 | — | | | |

XX: strong signal with OD>1.5, X: strong signal with 0.5<OD<1.5, −: OD<0.5 when 653 pg/mL FGF21 was used. The average value with intact FGF21 and FAP-cleaved FGF21 was used to generate the table.

On the basis of the results provided in Table 3, antibodies mAb2, 3 and 13 were excluded from further analysis. The results from Table 3 placed antibodies mAb1, 4, 8, 9, 10 and 11 into three epitope bins (Table 5).

TABLE 5
Epitope binning.

| Epitope bin | mAb |
|---|---|
| 1 | 1, 10, 11 |
| 2 | 4, 9 |
| 3 | 8 |

Antibodies mAb1, 4, 8, 9, 10 and 11 were then tested in ELISA using intact human FGF21 (Cat. No. 2539-FG, R&D Systems) in a combinatorial manner. The absorbance values were plotted as a function of antibody concentration and the data were fitted to a three parameter equation to calculate the half maximal effective concentration (EC₅₀) values for each antibody using Prism 6 (GraphPad Software, Inc., La Jolla, Calif.) (Table 6). As shown in Table 6, better potency was observed when antibodies mAb4 or 9 were used as the capture antibody and antibodies mAb10 or 11 were used as the detector antibody for intact human FGF21.

TABLE 6
EC₅₀ values with various anti-FGF21
mAb combinations in sandwich ELISA.

| Capture mAb | Detector mAb | EC₅₀ (pg/ml) |
|---|---|---|
| 4 | 10 | 108 |
| 4 | 11 | 133 |
| 9 | 11 | 156 |
| 9 | 10 | 161 |
| 11 | 4 | 161 |
| 10 | 4 | 172 |
| 8 | 10 + 4 | 182 |
| 10 | 9 | 191 |
| 4 | 1 | 193 |
| 11 | 9 | 195 |
| 8 | 11 + 4 | 198 |
| 8 | 1 + 4 | 202 |
| 1 | 4 | 222 |
| 8 | 4 | 228 |
| 8 | 10 | 237 |
| 9 | 1 | 249 |
| 8 | 11 | 303 |
| 8 | 1 | 308 |
| 1 | 9 | 323 |

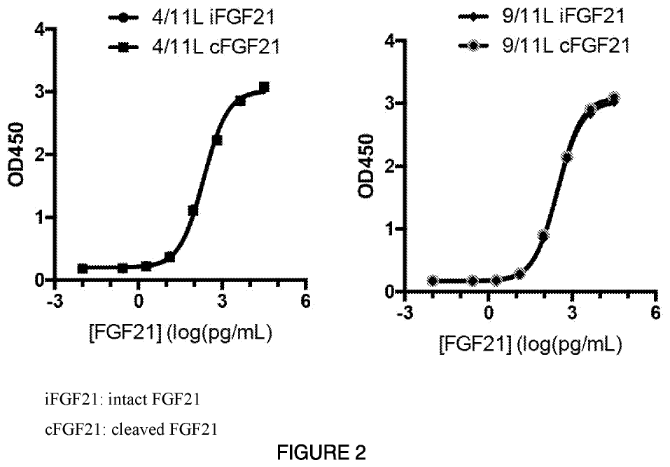

Antibodies mAb4, 8, 9, 10, 11, 15 and 16 were then tested in ELISA using intact human FGF21 (Cat. No. 2539-FG, R&D systems) or the FAP-cleaved human FGF21 in a combinatorial manner. The absorbance values were plotted as a function of antibody concentration and the data were fitted to a three parameter equation for each antibody using Prism 6 (GraphPad Software, Inc., La Jolla, Calif.). The most consistent result was observed when antibodies mAb4 or 9 were used as the capture antibody and antibody mAb11 or mAb15 was used as the detector antibody (FIG. 2 and Table 7). Therefore, mAb8 and 16 were removed from further analysis. FIG. 2 shows that the antibodies bind equally to intact and FAP-cleaved FGF21 (cFGF21), which is important to detect the concentration of total FGF21 (i.e., both intact and FAP-cleaved).

TABLE 7
EC₅₀ values with various anti-FGF21 mAb combinations in sandwich ELISA.

| Capture | Detection | EC₅₀ (pg/ml) with | EC₅₀ (pg/ml) with |
|---|---|---|---|
| mAb | mAb | intact FGF21 | FAP-cleaved FGF21 |
| 9 | 11 | 165.8 | 156.3 |
| 4 | 11 | 194.7 | 148 |
| 11 | 4 | 204.5 | 203.8 |
| 4 | 15 | 232.1 | 262.6 |
| 9 | 15 | 431.4 | 362.6 |
| 15 | 4 | 536.8 | 561.7 |
| 11 | 15 | 774.4 | 630.1 |
| 4 | 16 | 1246 | 7044 |
| 15 | 11 | 1388 | 1239 |
| 9 | 16 | 1451 | 6234 |
| 16 | 4 | 4893 | 15750 |
| 8 | 11 | 40272 | 106195 |
| 8 | 15 | 42549 | 78857 |
| 8 | 4 | 44411 | 121300000 |

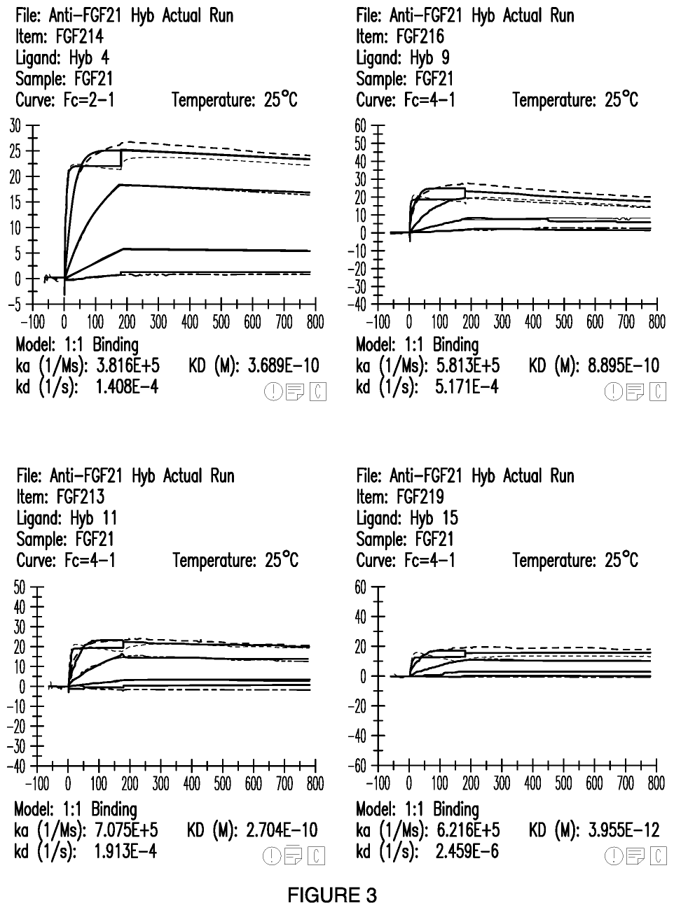

Antibodies mAb4, 9, 11 and 15 were further analyzed by BIACORE® surface plasmon resonance to determine the Kₐ. As shown in FIG. 3, mAb4 has a K_{d} of 3.689×10¹⁰, mAb9 has a K_{d} of 8.895×10¹⁰, mAb11 has a K_{d} of 2.704×10¹⁰ and mAb15 has a K_{d} of 3.955×10¹².

### Example 3: Epitope Analysis

Epitope mapping was conducted by expressing FGF19, FGF21 or FGF19-FGF21 chimeric proteins as FLAG-tagged proteins in transiently transfected HEK293 culture supernatant and testing the binding of antibodies mAb4, 9, 11 and 15 by ELISA. For ELISA, 96 well MaxiSorp plate (439454, Nalge Nunc International; Rochester, N.Y.) was coated with a mixture of 15 μl culture supernatant containing secreted protein and 135 μl of 1× coating buffer (50 mM sodium carbonate, pH 9.6) overnight at 4° C. Commercial antibodies R5 and R9, which bind to the C-terminus of FGF21, were used as positive controls.

Human FGF19:
(SEQ ID NO: 2)
RPLAFSDAGPHVHYGWGDPIRLRHLYTSGPHGLSSCFLRIRADGVVDCARGQSA
HSLLEIKAVALRTVAIKGVHSVRYLCMGADGKMQGLLQYSEEDCAFEEEIRPDG
YNVYRSEKHRLPVSLSSAKQRQLYKNRGFLPLSHFLPMLPMVPEEPEDLRGHLE
SDMFSSPLETDSMDPFGLVTGLEAVRSPSFEK
Human FGF21:
(SEQ ID NO: 1)
HPIPDSSPLLQFGGQVRQRYLYTDDAQQTEAHLEIREDGTVGGAADQSPESLLQL
KALKPGVIQILGVKTSRFLCQRPDGALYGSLHFDPEACSFRELLLEDGYNVYQSE
AHGLPLHLPGNKSPHRDPAPRGPARFLPLPGLPPALPEPPGILAPQPPDVGSSDPL
SMVGPSQGRSPSYAS
Human FGF21-19 chimera proteins (the FGF21 portion is
italicized and the FGF19 portion is underlined):
(SEQ ID NO: 3)
HPIPDSSPHVHYGWGDPIRLRHLYTSGPHGLSSCFLRIRADGVVDCARGQSAHSL
LEIKAVALRTVAIKGVHSVRYLCMGADGKMQGLLQYSEEDCAFEEEIRPDGYN
VYRSEKHRLPVSLSSAKQRQLYKNRGFLPLSHFLPMLPMVPEEPEDLRGHLESD
MFSSPLETDSMDPFGLVTGLEAVRSPSFEK
(SEQ ID NO: 4)
HPIPDSSPLLQFGGQVRQRYLYTDDAQQTEAHLEIRADGVVDCARGQSAHSLLEIK
AVALRTVAIKGVHSVRYLCMGADGKMQGLLQYSEEDCAFEEEIRPDGYNVYRS
EKHRLPVSLSSAKQRQLYKNRGFLPLSHFLPMLPMVPEEPEDLRGHLESDMFSSP
LETDSMDPFGLVTGLEAVRSPSFEK
(SEQ ID NO: 5)
HPIPDSSPLLQFGGQVRQRYLYTDDAQQTEAHLEIREDGVVDCARGQSAHSLLEIK
AVALRTVAIKGVHSVRYLCMGADGKMQGLLQYSEEDCAFEEEIRPDGYNVYRS
EKHRLPVSLSSAKQRQLYKNRGFLPLSHFLPMLPMVPEEPEDLRGHLESDMFSSP
LETDSMDPFGLVTGLEAVRSPSFEK
(SEQ ID NO: 6)
HPIPDSSPLLQFGGQVRQRYLYTDDAQQTEAHLEIREDGTVGGAADQSAHSLLEIKA
VALRTVAIKGVHSVRYLCMGADGKMQGLLQYSEEDCAFEEEIRPDGYNVYRSE
KHRLPVSLSSAKQRQLYKNRGFLPLSHFLPMLPMVPEEPEDLRGHLESDMFSSPL
ETDSMDPFGLVTGLEAVRSPSFEK
(SEQ ID NO: 7)
HPIPDSSPLLQFGGQVRQRYLYTDDAQQTEAHLEIREDGTVGGAADQSPESLLEIKA
VALRTVAIKGVHSVRYLCMGADGKMQGLLQYSEEDCAFEEEIRPDGYNVYRSE
KHRLPVSLSSAKQRQLYKNRGFLPLSHFLPMLPMVPEEPEDLRGHLESDMFSSPL
ETDSMDPFGLVTGLEAVRSPSFEK
(SEQ ID NO: 8)
HPIPDSSPLLQFGGQVRQRYLYTDDAQQTEAHLEIREDGTVGGAADQSPESLLQLKA
LKPGVIQILGVHSVRYLCMGADGKMQGLLQYSEEDCAFEEEIRPDGYNVYRSEK
HRLPVSLSSAKQRQLYKNRGFLPLSHFLPMLPMVPEEPEDLRGHLESDMFSSPLE
TDSMDPFGLVTGLEAVRSPSFEK
(SEQ ID NO: 9)
HPIPDSSPLLQFGGQVRQRYLYTDDAQQTEAHLEIREDGTVGGAADQSPESLLQLKA
LKPGVIQILGVKTSRFLCMGADGKMQGLLQYSEEDCAFEEEIRPDGYNVYRSEKH
RLPVSLSSAKQRQLYKNRGFLPLSHFLPMLPMVPEEPEDLRGHLESDMFSSPLET
DSMDPFGLVTGLEAVRSPSFEK
(SEQ ID NO: 10)
HPIPDSSPLLQFGGQVRQRYLYTDDAQQTEAHLEIREDGTVGGAADQSPESLLQLKA
LKPGVIQILGVKTSRFLCQRPDGALYGSLHFDPEACSFRELLLEDGYNVYQSEAHGLP
LHLPGNKSPHRDPAPRGPARFLPMLPMVPEEPEDLRGHLESDMFSSPLETDSMDP
FGLVTGLEAVRSPSFEK
(SEQ ID NO: 11)
RPLAFSDAGPLLQFGGQVRQRYLYTDDAQQTEAHLEIREDGTVGGAADQSPESLLQ
LKALKPGVIQILGVKTSRFLCQRPDGALYGSLHFDPEACSFRELLLEDGYNVYQSEAH
GLPLHLPGNKSPHRDPAPRGPARFLPLPGLPPALPEPPGILAPQPPDVGSSDPLSMV
GPSQGRSPSYAS
(SEQ ID NO: 12)
RPLAFSDAGPHVHYGWGDPIRLRHLYTSGPHGLSSCFLRIREDGTVGGAADQSPE
SLLQLKALKPGVIQILGVKTSRFLCQRPDGALYGSLHFDPEACSFRELLLEDGYNVYQ
SEAHGLPLHLPGNKSPHRDPAPRGPARFLPLPGLPPALPEPPGILAPQPPDVGSSDPL
SMVGPSQGRSPSYAS
(SEQ ID NO: 13)
RPLAFSDAGPHVHYGWGDPIRLRHLYTSGPHGLSSCFLRIRADGTVGGAADQSPE
SLLQLKALKPGVIQILGVKTSRFLCQRPDGALYGSLHFDPEACSFRELLLEDGYNVYQ
SEAHGLPLHLPGNKSPHRDPAPRGPARFLPLPGLPPALPEPPGILAPQPPDVGSSDPL
SMVGPSQGRSPSYAS
(SEQ ID NO: 14)
RPLAFSDAGPHVHYGWGDPIRLRHLYTSGPHGLSSCFLRIRADGVVDCARGQSP
ESLLQLKALKPGVIQILGVKTSRFLCQRPDGALYGSLHFDPEACSFRELLLEDGYNVY
QSEAHGLPLHLPGNKSPHRDPAPRGPARFLPLPGLPPALPEPPGILAPQPPDVGSSD
PLSMVGPSQGRSPSYAS
(SEQ ID NO: 15)
RPLAFSDAGPHVHYGWGDPIRLRHLYTSGPHGLSSCFLRIRADGVVDCARGQSA
HSLLQLKALKPGVIQILGVKTSRFLCQRPDGALYGSLHFDPEACSFRELLLEDGYNVY
QSEAHGLPLHLPGNKSPHRDPAPRGPARFLPLPGLPPALPEPPGILAPQPPDVGSSD
PLSMVGPSQGRSPSYAS
(SEQ ID NO: 16)
RPLAFSDAGPHVHYGWGDPIRLRHLYTSGPHGLSSCFLRIRADGVVDCARGQSA
HSLLEIKAVALRTVAIKGVHSVRYLCQRPDGALYGSLHFDPEACSFRELLLEDGYN
VYQSEAHGLPLHLPGNKSPHRDPAPRGPARFLPLPGLPPALPEPPGILAPQPPDVGSS
DPLSMVGPSQGRSPSYAS
(SEQ ID NO: 17)
RPLAFSDAGPHVHYGWGDPIRLRHLYTSGPHGLSSCFLRIRADGVVDCARGQSA
HSLLEIKAVALRTVAIKGVHSVRYLCMGADGKMQGLLQYSEEDCAFEEEIRPDG
YNVYRSEKHRLPVSLSSAKQRQLYKNRGFLPLSHFLPLPGLPPALPEPPGILAPQP
PDVGSSDPLSMVGPSQGRSPSYAS

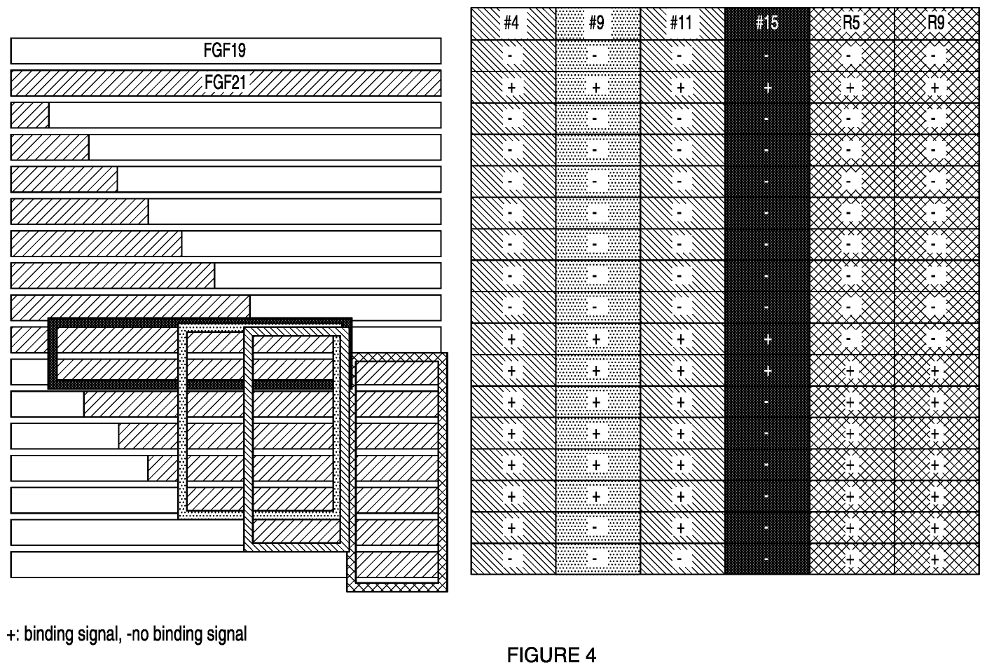

As shown in FIG. 4, antibodies mAb4, 9, 11 and 15 bind to the core FGF folds of human FGF21 and do not bind to the N-terminal or C-terminal flexible regions.

### Example 4: FGF21 ELISA Assay

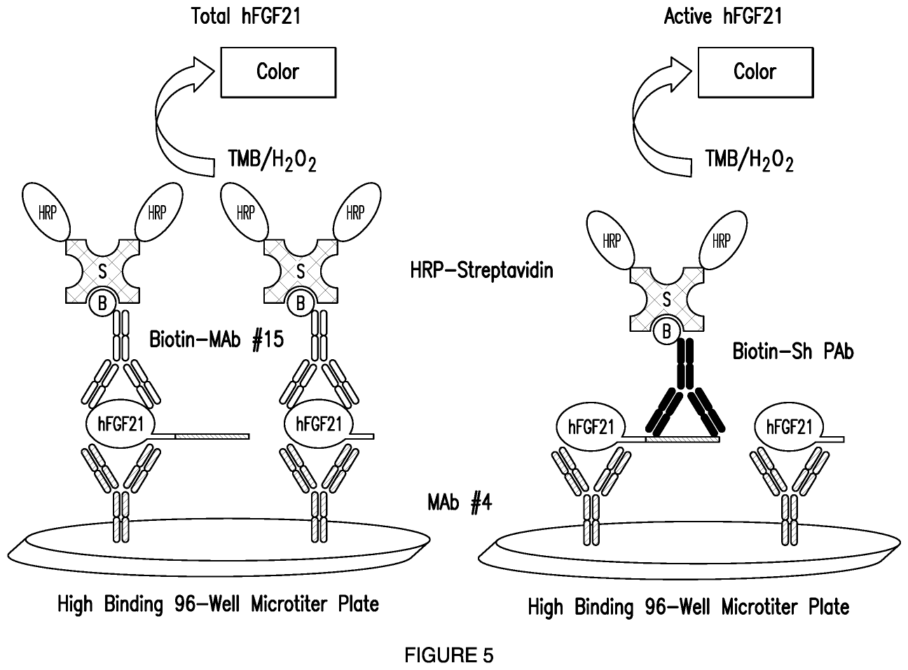

The utility of antibodies mAb4 and 11 as capture antibodies in the detection of intact FGF21 was tested in combination with a C-terminal specific anti-FGF21 pAb (Cat. No. 30661, Epitope Diagnostics, San Diego, Calif.; also referred to herein as “C-ter pAb”)) biotinylated by using EZ-Link™ NHS-PEG Solid-Phase Biotinylation Kit (PIERCE #21450). A schematic of the immunoassays to determine total FGF21 and active FGF21 levels is shown in FIG. 5.

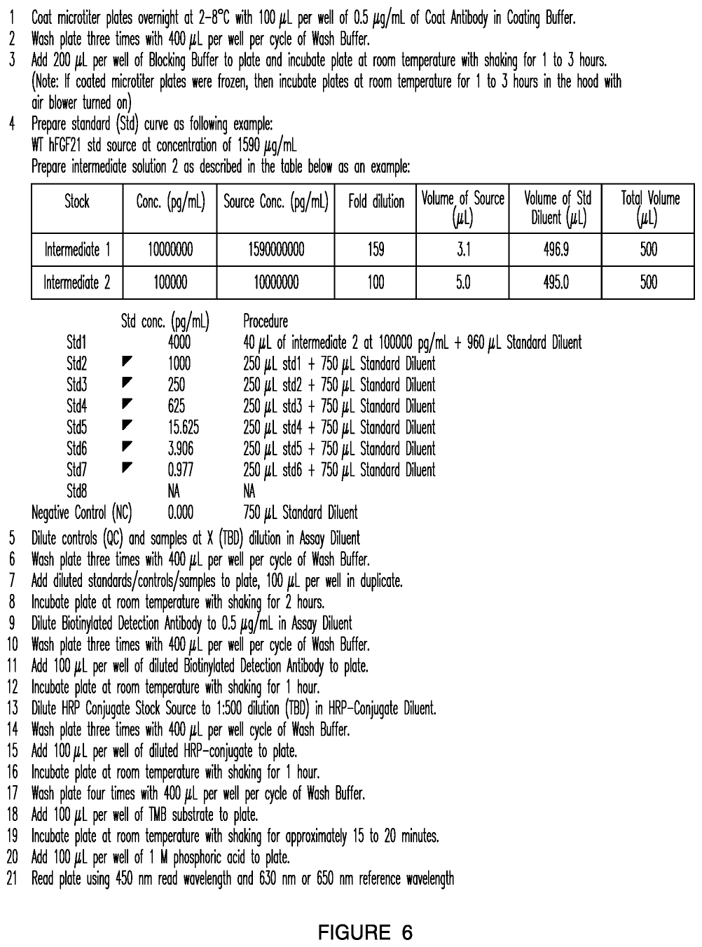

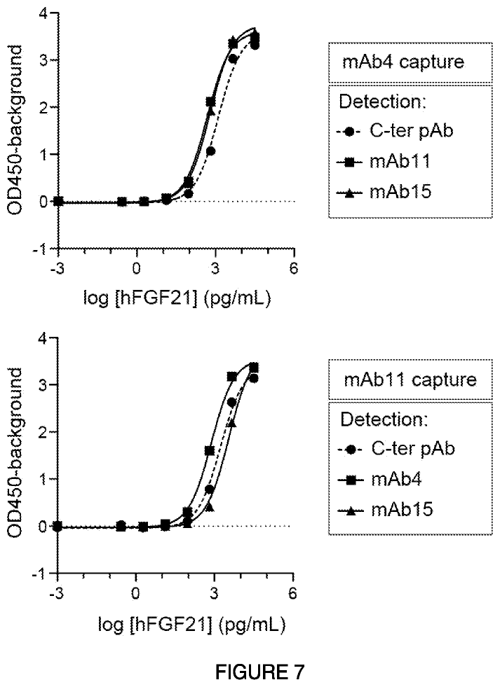

The ELISA assay was performed as follows: 96 well MaxiSorp plate (Cat. No. 439454, Nalge Nunc International; Rochester, N.Y.) was coated with 0.5 μg/mL of anti-FGF21 mAbs in coating buffer (50 mM sodium carbonate, pH 9.6) overnight at 4° C. On the next day, after blocking with PBS containing 0.5% BSA and 10 ppm Proclin pH 7.4, and washing with Washing buffer (PBS, 0.05% Tween 20, pH7.2), plate was incubated with 0.0004-32000 pg/mL of intact human FGF21 (2539-FG, R&D systems) in assay buffer (25 mM HEPES, pH 7.2, 150 mM NaCl, 0.2 mM CaCl₂, 0.1% bovine serum albumin [BSA], 0.05% Tween-20) for 1-2 h at room temperature. After washing with Washing buffer, the plate was incubated with 0.5 μg/ml of the secondary Ab (biotinylated anti-FGF21 C-terminal pAb 30661 or anti-FGF21 mAb11 or 15) in Magic buffer (1×PBS pH 7.4, 0.5% BSA, 0.05% Tween 20, 0.2% BgG, 0.25% CHAPS, 5 mM EDTA, 0.35M NaCl, 10 PPM Proclin) for 1-2 hr at room temperature. After washing with Washing buffer, plate was incubated with High Sensitivity Streptavidin-HRP (PIERCE #21130) diluted 1:1,000 in assay buffer. After washing with Washing buffer, the binding of anti-FGF21 to recombinant FGF21 was assessed by adding substrate 3, 3′, 5, 5′-tetramethyl benzidine (TMBE-1000, Moss; Pasadena, Md.). A more detailed protocol is provided in FIG. 6. The mean absorbance values from duplicate wells were plotted as a function of antibody concentration and the data were fitted to a three-parameter equation using Prism 6 (GraphPad Software, Inc., La Jolla, Calif.) (FIG. 7).

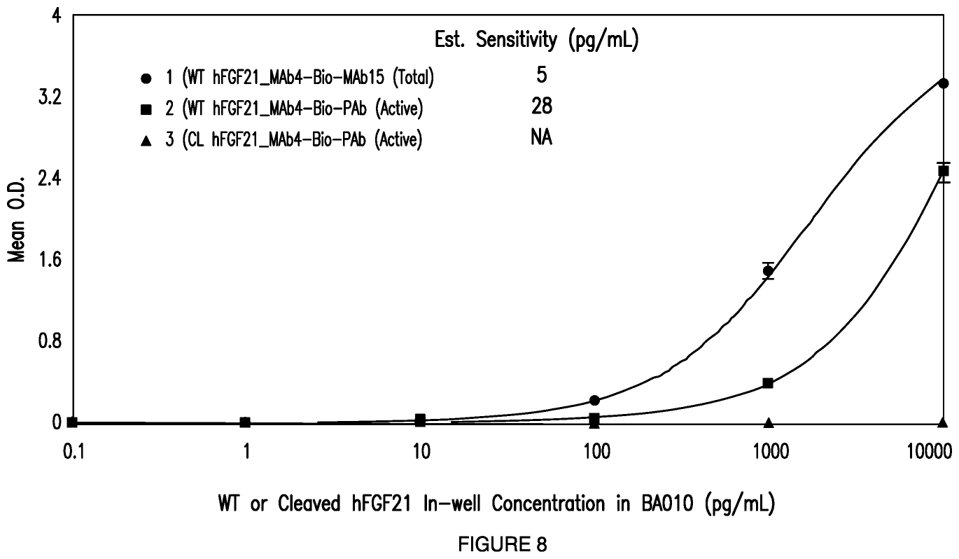

As shown in FIG. 8, the total FGF21 ELISA assay had an in-well sensitivity of 5 pg/ml and the active FGF21 ELISA assay had an in-well sensitivity of 28 pg/ml. The active FGF21 ELISA assay did not detect the cleaved form of FGF21 that is missing the last 10 C-terminal amino acids.

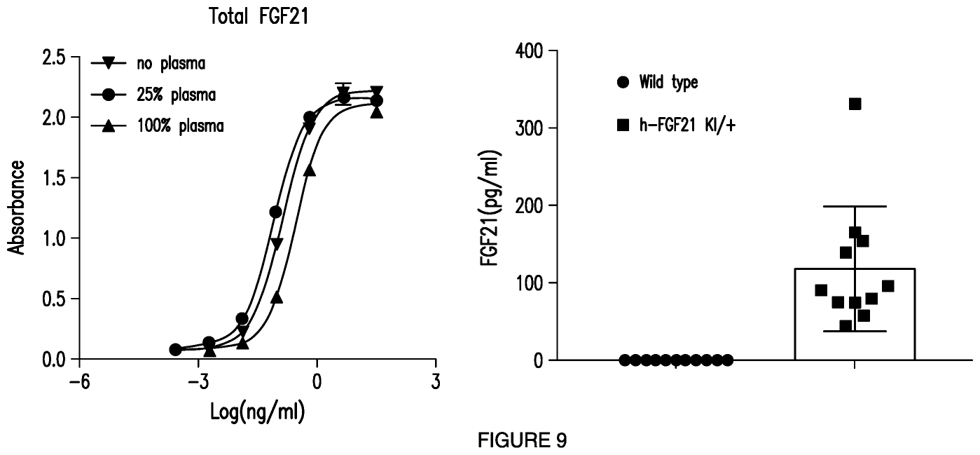

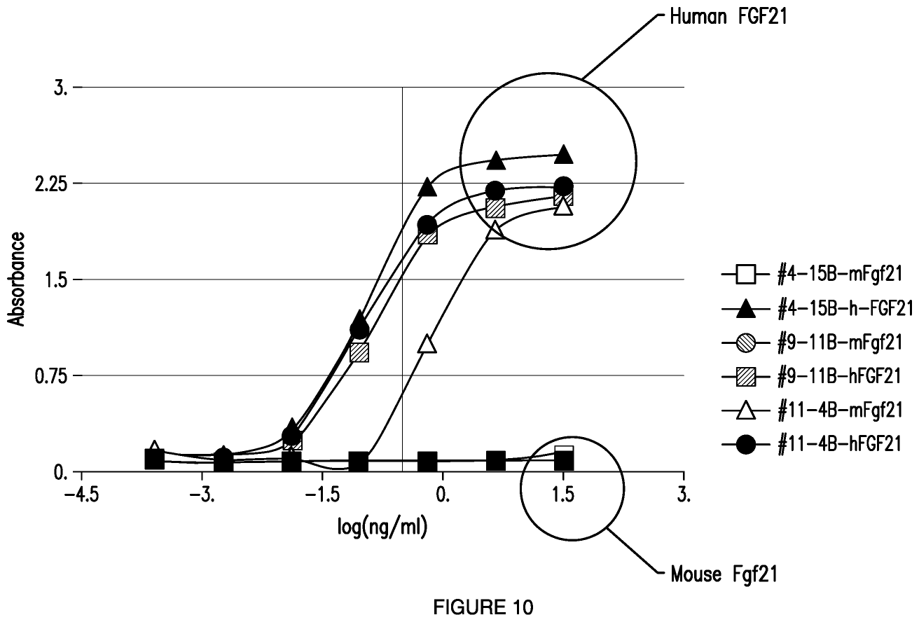

Further experiments were performed to determine the effect of serum has on the total FGF21 ELISA assay. FGF21 ELISA assays using mAb4 as the capture antibody and mAb15 at the detector antibody were performed. As shown in FIG. 9, there was minimal serum interference on the assay. The specificity of the assay for human FGF21 was also tested. As shown in FIG. 9, the assay for total FGF21 detected human FGF21 that was expressed in human-FGF21 knock-in mice as compared to control mice. FIG. 10 also shows that the assay using the disclosed antibodies was specific for human FGF21 and did not detect mouse FGF21.

On the basis of these data, 4 antibodies, mAb4, mAb9, mAb11 and mAb15, were chosen for cDNA cloning for recombinant expression. The amino acid sequences of these antibodies are provided in Tables 8-13 and FIGS. 41A and 41B. Recombinant mAbs were expressed in 100 mL CHO culture in the murine IgG2a background.

TABLE 8
Full-length light chain (LC) sequences for murine
anti-FGF21 monoclonal antibodies.

| Antibody | Full-length Light Chain Amino Acid Sequence |
|---|---|
| 4 | QIVLTQSPAIMSAPLGERVTMTCTASSSVSSSYLHWYQQKPGSSPKVWIYRT |
| | TNLASGVPTRFSGSGSGTSYSLTISSMEAEDAATYYCHQYHRSPPTWTFGGG |
| | TKLEIKRADAAPTVSIFPPSSEQLTSGGASVVCFLNNFYPKDINVKWKIDGSE |
| | RQNGVLNSWTDQDSKDSTYSMSSTLTLTKDEYERHNSYTCEATHKTSTSPI |
| | VKSFNRNEC (SEQ ID NO: 18) |
| 9 | DIQMTQSPASLSASVGETVIITCRASENIYSYLAWYQQKQGKSPQLLVYNIRT |
| | LAEGVPSRFSGSGSGTQFSLKINSLQPEDFGSYYCQHHYDSPWTFGGGTKLEI |
| | KRADAAPTVSIFPPSSEQLTSGGASVVCFLNNFYPKDINVKWKIDGSERQNG |
| | VLNSWTDQDSKDSTYSMSSTLTLTKDEYERHNSYTCEATHKTSTSPIVKSFN |
| | RNEC (SEQ ID NO: 19) |
| 11 | QIVLTQSPALMSASPGERVTMTCSAGSSVSYMYWYQQKPRSSPKPWIYLTS |
| | NLASGVPARFSGSGSGTSYSLTISSMEAEDAATYYCQQWSSNPRTFGGGTKL |
| | EIKRADAAPTVSIFPPSSEQLTSGGASVVCFLNNFYPKDINVKWKIDGSERQN |
| | GVLNSWTDQDSKDSTYSMSSTLTLTKDEYERHNSYTCEATHKTSTSPIVKSF |
| | NRNEC (SEQ ID NO: 20) |
| 15 | DVLMTQTPLSLPVSLGDQASISCRSSQIIVHNNGDTYLEWYLQKPGQSPKLLI |
| | YKISNRFSGVPDRFSGSGSGTDFTLKISRVEAEDLGVYYCFQGSHVPYTFGG |
| | GTKLEIKRADAAPTVSIFPPSSEQLTSGGASVVCFLNNFYPKDINVKWKIDGS |
| | ERQNGVLNSWTDQDSKDSTYSMSSTLTLTKDEYERHNSYTCEATHKTSTSPI |
| | VKSFNRNEC (SEQ ID NO: 21) |

TABLE 9
Full-length heavy chain (HC) sequences for murine
anti-FGF21 monoclonal antibodies.

| Antibody | Full-length Heavy Chain Amino Acid Sequence |
|---|---|
| 4 | EVKLVESGGDLVKPGGSLKLSCAASGFTFSSYGMSWVRQTPDKRLEWVATI |
| | STGGGYTYYPDSVKGRFTISRDNAKNTLYLQMSSLRSEDTAMYYCARHDL |
| | VDWYFDVWGTGTTVTVSSAKTTAPSVYPLAPVCGDTTGSSVTLGCLVKGY |
| | FPEPVTLTWNSGSLSSGVHTFPAVLQSDLYTLSSSVTVTSSTWPSQSITCNVA |
| | HPASSTKVDKKIEPRGPTIKPCPPCKCPAPNLLGGPSVFIFPPKIKDVLMISLSP |
| | IVTCVVVDVSEDDPDVQISWFVNNVEVHTAQTQTHREDYNSTLRVVSALPI |
| | QHQDWMSGKEFKCKVNNKDLPAPIERTISKPKGSVRAPQVYVLPPPEEEMT |
| | KKQVTLTCMVTDFMPEDIYVEWTNNGKTELNYKNTEPVLDSDGSYFMYSK |
| | LRVEKKNWVERNSYSCSVVHEGLHNHHTTKSFSRTPGK (SEQ ID NO: 22) |
| 9 | EVQLQQSGPELVKPGASVKIPCKASGYTFTDYYMGWVKQSHGKSLEWIGDI |
| | NPNNGVTINNQNFKGKATLTVDKSSSTAYMELRSLASEDTAVYYCTRGYG |
| | GALDYWGQGTSVTVSSAKTTAPSVYPLAPVCGDTTGSSVTLGCLVKGYFPE |
| | PVTLTWNSGSLSSGVHTFPAVLQSDLYTLSSSVTVTSSTWPSQSITCNVAHPA |
| | SSTKVDKKIEPRGPTIKPCPPCKCPAPNLLGGPSVFIFPPKIKDVLMISLSPIVT |
| | CVVVDVSEDDPDVQISWFVNNVEVHTAQTQTHREDYNSTLRVVSALPIQHQ |
| | DWMSGKEFKCKVNNKDLPAPIERTISKPKGSVRAPQVYVLPPPEEEMTKKQ |
| | VTLTCMVTDFMPEDIYVEWTNNGKTELNYKNTEPVLDSDGSYFMYSKLRV |
| | EKKNWVERNSYSCSVVHEGLHNHHTTKSFSRTPGK (SEQ ID NO: 23) |
| 11 | QVQLQQSGAELARPGASVKLSCKASGYTFTNYGISWVKQRTGQGLEWIGEI |
| | YPRSDNTYYNEKFKGKATLTADKSSSTAYMELRSLTSEDSAVYFCTRSDYG |
| | FFDYWGQGTTLTVSSAKTTAPSVYPLAPVCGDTTGSSVTLGCLVKGYFPEP |
| | VTLTWNSGSLSSGVHTFPAVLQSDLYTLSSSVTVTSSTWPSQSITCNVAHPAS |
| | STKVDKKIEPRGPTIKPCPPCKCPAPNLLGGPSVFIFPPKIKDVLMISLSPIVTC |
| | VVVDVSEDDPDVQISWFVNNVEVHTAQTQTHREDYNSTLRVVSALPIQHQD |
| | WMSGKEFKCKVNNKDLPAPIERTISKPKGSVRAPQVYVLPPPEEEMTKKQV |
| | TLTCMVTDFMPEDIYVEWTNNGKTELNYKNTEPVLDSDGSYFMYSKLRVE |
| | KKNWVERNSYSCSVVHEGLHNHHTTKSFSRTPGK (SEQ ID NO: 24) |
| 15 | QVQLIQSGPGLVQPSQSLSITCTVSGFSLTGYAIHWVRQSPGKGLEWLGMIW |
| | KSGNTDYNAAFMSRLSITKDNSKSQVFFKMNSLQADDTAIYYCARNGYDY |
| | EFVYWGQGTLVTVSAAKTTAPSVYPLAPVCGDTTGSSVTLGCLVKGYFPEP |
| | VTLTWNSGSLSSGVHTFPAVLQSDLYTLSSSVTVTSSTWPSQSITCNVAHPAS |
| | STKVDKKIEPRGPTIKPCPPCKCPAPNLLGGPSVFIFPPKIKDVLMISLSPIVTC |
| | VVVDVSEDDPDVQISWFVNNVEVHTAQTQTHREDYNSTLRVVSALPIQHQD |
| | WMSGKEFKCKVNNKDLPAPIERTISKPKGSVRAPQVYVLPPPEEEMTKKQV |
| | TLTCMVTDFMPEDIYVEWTNNGKTELNYKNTEPVLDSDGSYFMYSKLRVE |
| | KKNWVERNSYSCSVVHEGLHNHHTTKSFSRTPGK (SEQ ID NO: 25) |

TABLE 10
Light Chain Variable Region (VL) sequences for
murine anti-FGF21 monoclonal antibodies.

| Antibody | Light Chain Variable Region Amino Acid Sequence |
|---|---|
| 4 | QIVLTQSPAIMSAPLGERVTMTCTASSSVSSSYLHWYQQKPGSSPKVWIYRT |
| | TNLASGVPTRFSGSGSGTSYSLTISSMEAEDAATYYCHQYHRSPPTWTFGGG |
| | TKLEIK (SEQ ID NO: 50) |
| 9 | DIQMTQSPASLSASVGETVIITCRASENIYSYLAWYQQKQGKSPQLLVYNIRT |
| | LAEGVPSRFSGSGSGTQFSLKINSLQPEDFGSYYCQHHYDSPWTFGGGTKLEI |
| | K (SEQ ID NO: 51) |
| 11 | QIVLTQSPALMSASPGERVTMTCSAGSSVSYMYWYQQKPRSSPKPWIYLTS |
| | NLASGVPARFSGSGSGTSYSLTISSMEAEDAATYYCQQWSSNPRTFGGGTKL |
| | EIK (SEQ ID NO: 52) |
| 15 | DVLMTQTPLSLPVSLGDQASISCRSSQIIVHNNGDTYLEWYLQKPGQSPKLLI |
| | YKISNRFSGVPDRFSGSGSGTDFTLKISRVEAEDLGVYYCFQGSHVPYTFGG |
| | GTKLEIK (SEQ ID NO: 53) |

TABLE 11
Heavy Chain Variable Region (VH) sequences for
murine anti-FGF21 monoclonal antibodies.

| Antibody | Heavy Chain Variable Region Amino Acid Sequence |
|---|---|
| 4 | EVKLVESGGDLVKPGGSLKLSCAASGFTFSSYGMSWVRQTPDKRLEWVATI |
| | STGGGYTYYPDSVKGRFTISRDNAKNTLYLQMSSLRSEDTAMYYCARHDL |
| | VDWYFDVWGTGTTVTVSS (SEQ ID NO: 54) |
| 9 | EVQL QQSGPELVKPGASVKIPCKASGYTFTDYYMGWVKQSHGKSLEWIGDI |
| | NPNNGVTINNQNFKGKATLTVDKSSSTAYMELRSLASEDTAVYYCTRGYG |
| | GALDYWGQGTSVTVSS (SEQ ID NO: 55) |
| 11 | QVQLQQSGAELARPGASVKLSCKASGYTFTNYGISWVKQRTGQGLEWIGEI |
| | YPRSDNTYYNEKFKGKATLTADKSSSTAYMELRSLTSEDSAVYFC TRSDYG |
| | FFDYWGQGTTLTVSS (SEQ ID NO: 56) |
| 15 | QVQLIQSGPGLVQPSQSLSITCTVSGFSLTGYAIHWVRQSPGKGLEWLGMIW |
| | KSGNTDYNAAFMSRLSITKDNSKSQVFFKMNSLQADDTAIYYCARNGYDY |
| | EFVYWGQGTLVTVSA (SEQ ID NO: 57) |

TABLE 12
Heavy Chain CDR sequences for murine anti-FGF21
monoclonal antibodies.

| Anti- | | | |
|---|---|---|---|
| body | CDR H1 | CDR H2 | CDR H3 |
| 4 | SYGMS (SEQ | TISTGGGYTYYPDSVKG | HDLVDWYFDV |
| | ID NO: 26) | (SEQ ID NO: 30) | (SEQ ID NO: 34) |
| 9 | DYYMG (SEQ | DINPNNGVTINNQNFKG | GYGGALDY (SEQ |
| | ID NO: 27) | (SEQ ID NO: 31) | ID NO: 35) |
| 11 | NYGIS (SEQ | EIYPRSDNTYYNEKFKG | SDYGFFDY (SEQ |
| | ID NO: 28) | (SEQ ID NO: 32) | ID NO: 36) |
| 15 | GYAIH (SEQ | MIWKSGNTDYNAAFMS | NGYDYEFVY (SEQ |
| | ID NO: 29) | (SEQ ID NO: 33) | ID NO: 37) |

TABLE 13
Light Chain CDR sequences for murine anti-FGF21
monoclonal antibodies.

| Anti- | | | |
|---|---|---|---|
| body | CDR L1 | CDR L2 | CDR L3 |
| 4 | TASSSVSSSYLH | RTTNLAS (SEQ | HQYHRSPPTWT |
| | (SEQ ID NO: 38) | ID NO: 42) | (SEQ ID NO: 46) |
| 9 | RASENIYSYLA | NIRTLAE (SEQ | QHHYDSPWT (SEQ |
| | (SEQ ID NO: 39) | ID NO: 43) | ID NO: 47) |
| 11 | SAGSSVSYMY | LTSNLAS (SEQ | QQWSSNPRT (SEQ |
| | (SEQ ID NO: 40) | ID NO: 44) | ID NO: 48) |
| 15 | RSSQIIVHNNGDTYLE | KISNRFS (SEQ | FQGSHVPYT |
| | (SEQ ID NO: 41) | ID NO: 45) | (SEQ ID NO: 49) |

### Example 5: Optimization of the FGF21 ELISA Assay

The FGF21 ELISA Assay described in Example 4 was further optimized to improve the sensitivity of the assay.

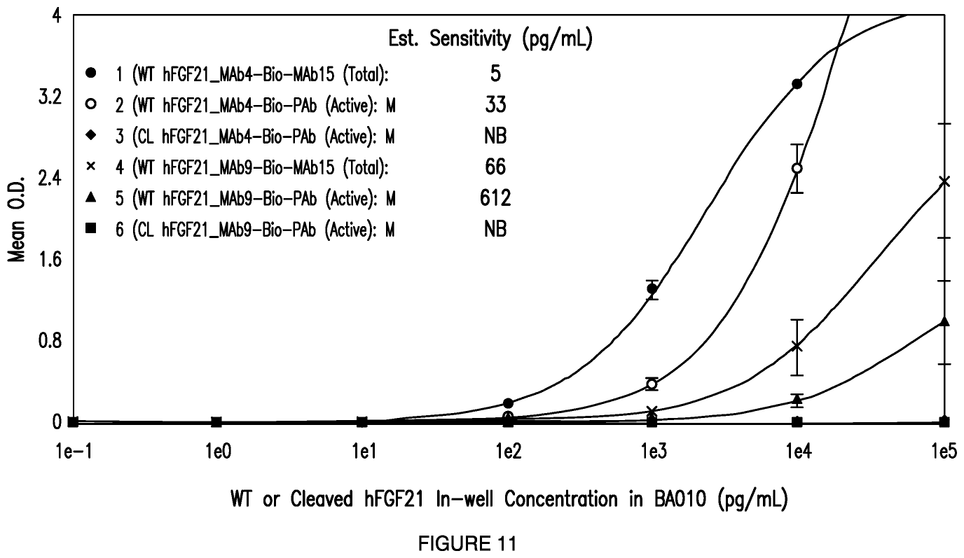

Different capture antibodies were compared to determine which capture antibody resulted in more superior detection. Antibodies mAb4 and mAb9 were both tested as the capture antibody. As shown in FIG. 11, better assay sensitivity was obtained using mAb4 as the capture antibody compared to mAb9.

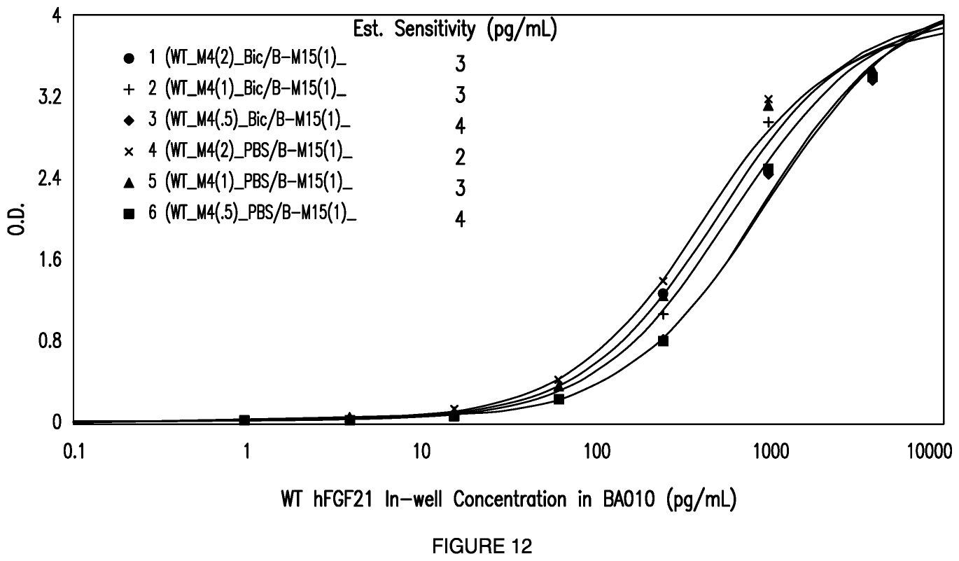

Different types of coating buffers and different concentrations of coating antibody at fix concentration of detecting antibody were analyzed for the total FGF21 assay and the active FGF21 assay. A bicarbonate coating buffer and a PBS coating buffer were analyzed at different coating antibody concentrations. As shown in FIG. 12, for the total FGF21 assay, similar in-well sensitivities were observed for sodium bicarbonate and PBS coating buffer, even at different concentrations of coating antibody. For example, coating 2 μg/ml of mAb4 in PBS had an in-well sensitivity of 2 pg/ml and coating 2 μg/ml of mAb4 had an in-well sensitivity of 3 pg/ml.

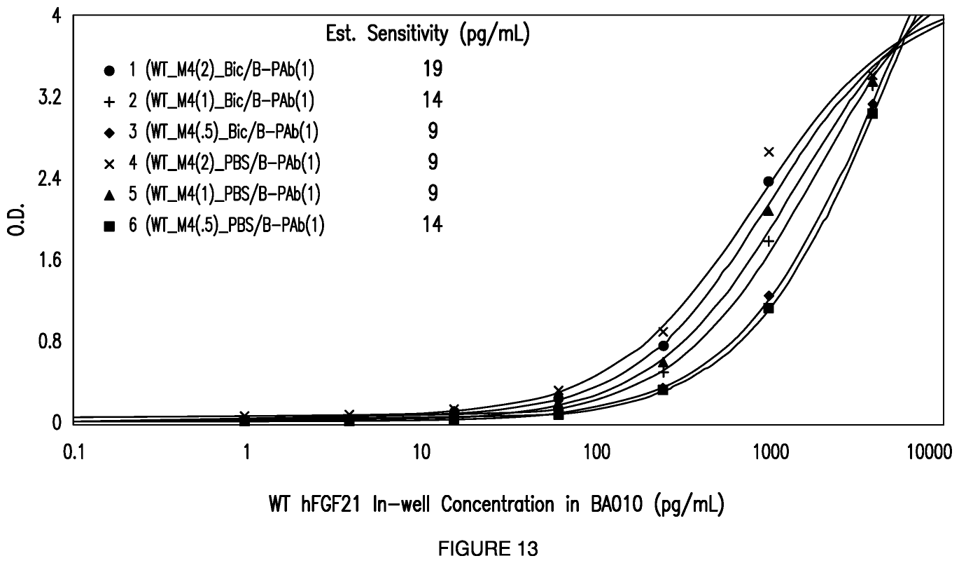

For the active FGF21 assay, similar in-well sensitivities were observed for sodium bicarbonate and PBS coating buffer (FIG. 13).

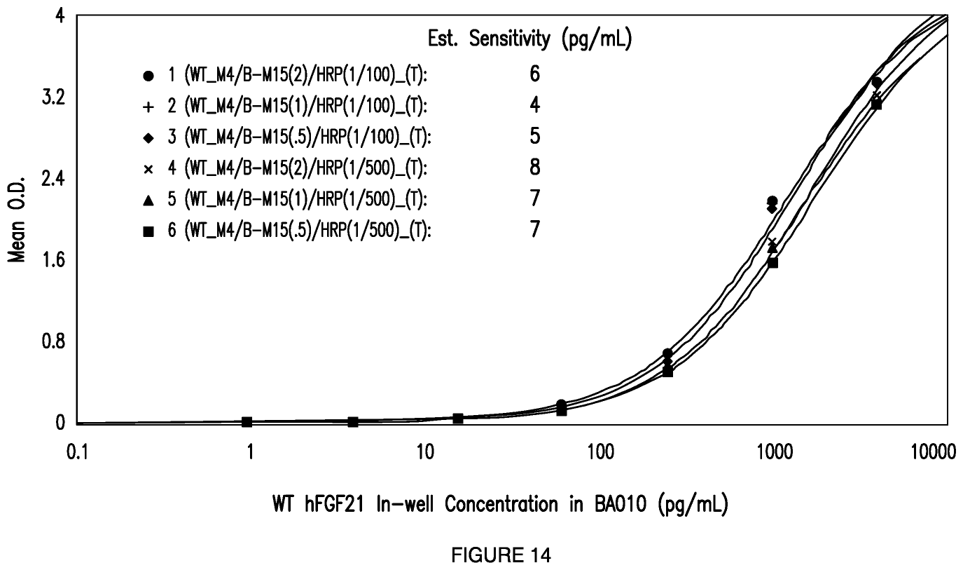

Additional experiments were performed to determine the effects the concentration of the detector antibody (mAb15) and the concentration of the horseradish peroxide (HRP) had on the sensitivity of the total FGF21 assay. Concentrations of 0.2, 1 and 2 μg/ml were tested for the detector antibody and dilution of 1/100 and 1/500 were tested for HRP. As shown in FIG. 14, higher concentrations of the detector antibody and HRP did not significantly improve the sensitivity of the assays.

### Example 6: FGF21 Detection Assays Using Quanterix Simoa

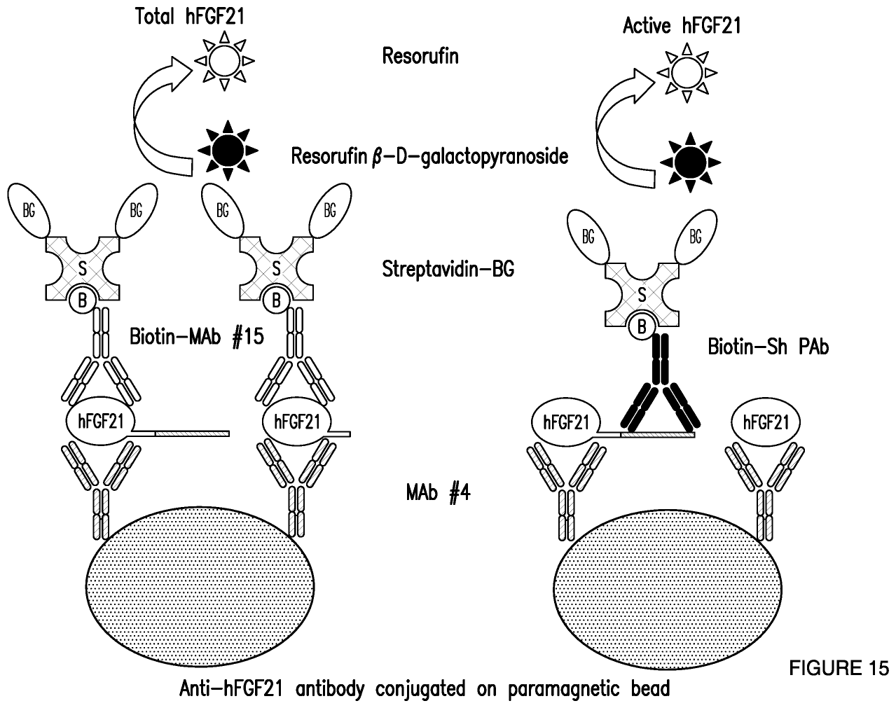

Based on optimizations from the ELISA format, as discussed in Example 5, an assay using the Quanterix Simoa HD-1 Analyzer™ was adapted to use mAb4 as the capture antibody, and either biotinylated mAb15 (to detect total FGF21) or biotinylated C-ter pAb (to detect active FGF21) as the detector antibodies. A schematic of the assay is shown in FIG. 15.

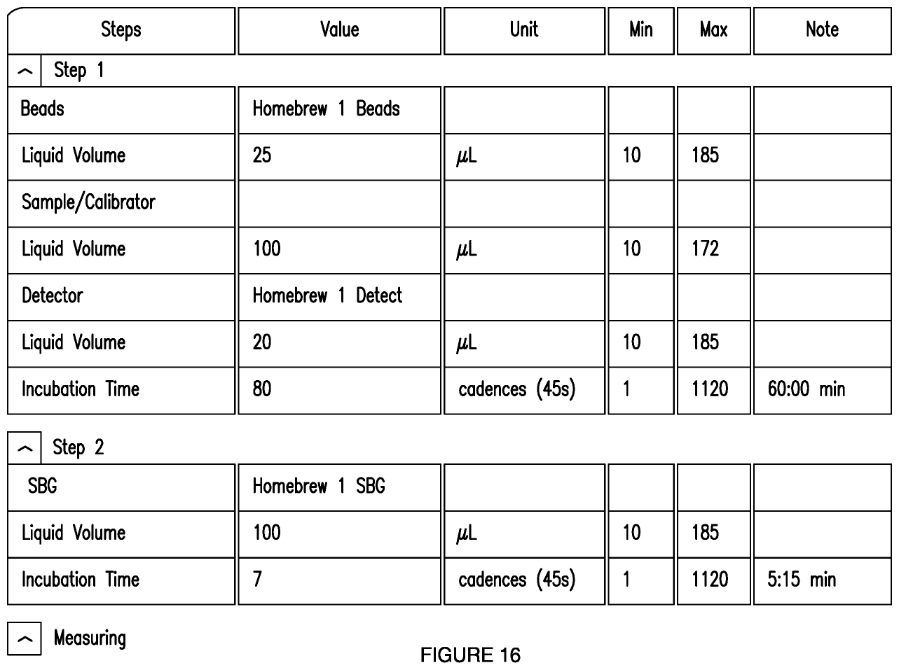

A summary of the immunoassay is provided. The Quanterix Simoa immunoassay starts with the capture and labeling of total FGF21 with an enzyme conjugate (streptavidin β-galactosidase (SBG)), using a 2-step assay protocol (FIG. 16). Total FGF21 captured with magnetic beads conjugated to mAb4 and biotinylated detection antibody (either mAb15-Biotin for total FGF21 or C-ter pAb-Biotin for active FGF21) are added together to form a captured analyte sandwich in the first step, then SBG is added for detection in the second step. Between each step, the beads are washed. During each wash cycle, the instrument uses a magnet to pellet beads before automated aspiration of the supernatant. After the final wash cycle, the capture beads are resuspended in resorufin β-D-galactopyranoside (RGP) substrate. The beads are then transferred to the entry port of a Simoa Disc in preparation for imaging and analyte quantification.

After capture and labeling of FGF21, the capture beads are loaded into an array containing 216,000 40-fL wells that have been sized to hold no more than one bead per well (4.25 μm width, 3.25 μm depth). The bead suspension is pulled through the entry channel and over the array. Beads are allowed to settle into the wells via gravity for approximately 90 seconds. An aliquot of oil is dispensed in the array entry channel and pulled over the array, trapping the beads and RGP substrate in the microwells as well as removing excess beads from the surface. If a FGF21 molecule has been captured and labeled, the SBG hydrolyzes RGP substrate into the fluorescent product resorufin. The fluorescent product builds up within the sealed microwells, enabling detection of single molecules.

The multiplex capture beads were prepared using a two-step EDAC coupling protocol (Simoa Homebrew 2.0 Multiplex Bead Coating Protocol USER-213-11). Beads are coupled with 0.5 mg/mL mAb4 and 0.25 mg/mL EDAC. The coupling reaction occurs between the antibody primary amino groups and the carboxyl groups on the beads.

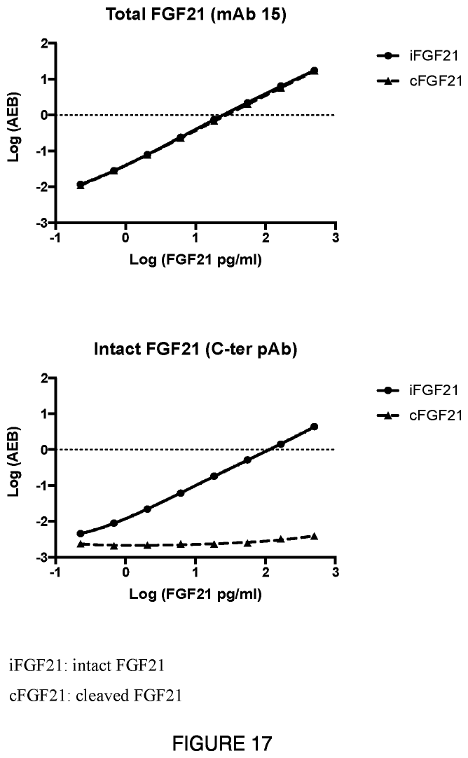

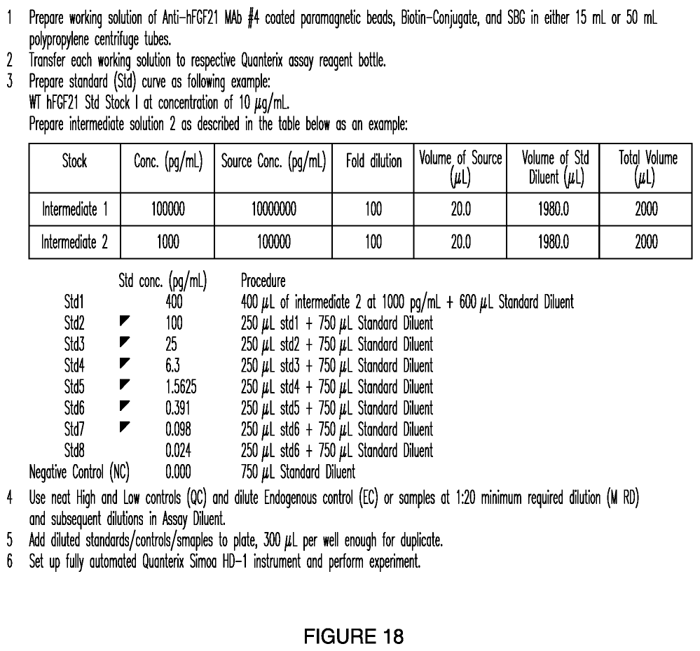

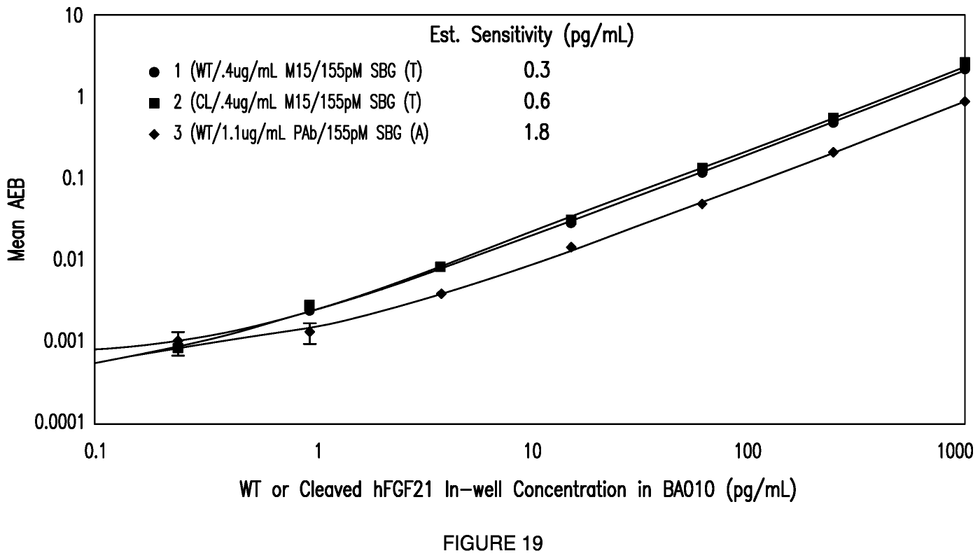

Quanterix Simoa assay was performed in 96-well Nunc™ 96-Well Polypropylene MicroWell™ Plates (V-bottom, Thermo Scientific Nunc 249944, Rochester, N.Y.). For the standard curve, recombinant intact human FGF21 (iFGF21) and cleaved human FGF21 (cFGF21) were serially diluted in Simoa buffer (PBS pH 7.4, 2% BSA (Fraction B, Protease-Free), 0.1% Tween, 5 mM EDTA) from 0.200-500 pg/mL (FIG. 17) or Magic Buffer (BA010) (FIGS. 19-25, 28-32 and 33-37). To determine the unknown concentration of FGF21 (e.g., in plasma or serum), the test samples were diluted at 1:5-1:20 in Simoa buffer or Magic Buffer. The assay plate, along with the required recommended reagents were loaded into the Simoa HD-1 Analyzer. In each well, for each reaction, 32 μL of capture beads conjugated to mAb 4, 32 μL of detector antibodies at 1 μg/mL (mAb15-Biotin or C-ter pAb-Biotin) and 110 μL of SBG were used. For each well, the assay was performed in duplicate. The manufacturer's default Homebrew Assay was selected as the program for the automated procedures. Additional information regarding the assay protocol is provided in FIG. 18.

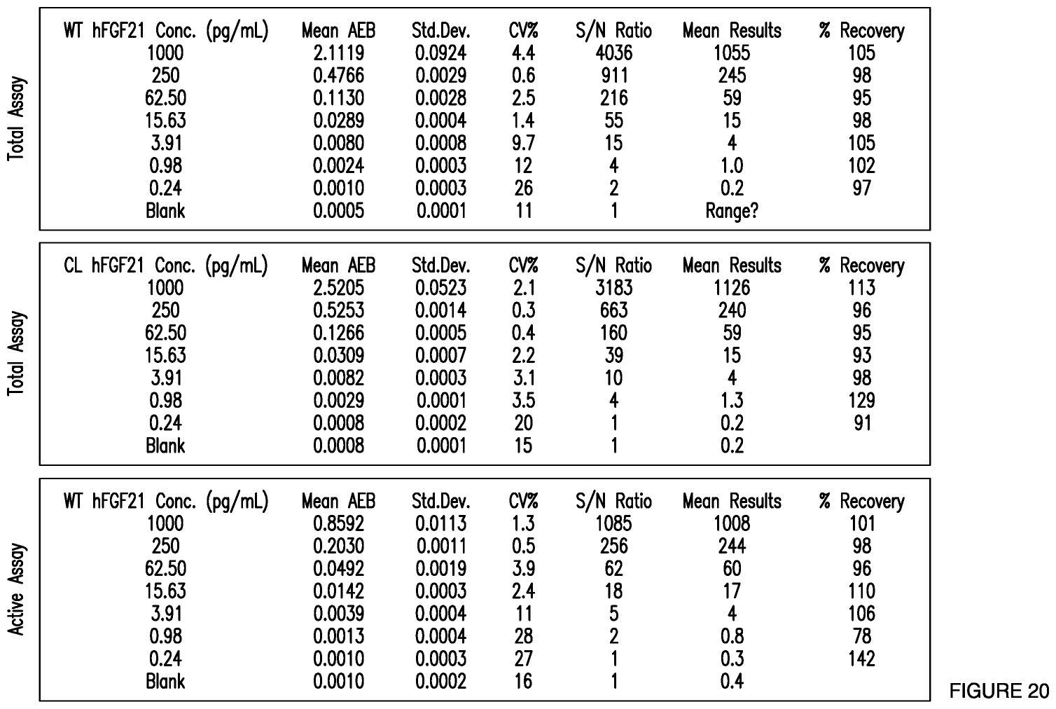

As shown in FIG. 19, the total (T) FGF21 Quanterix Simoa-based assay (QSA) detected intact (Wild Type (WT)) FGF21 with an in-well sensitivity (based on 2×mean AEB of blank wells) of 0.3 pg/ml and the cleaved (CL) form of FGF21, which does not have the last 10 C-terminal amino acids, with an in-well sensitivity of 0.6 pg/ml. The active (A) FGF21 QSA detected intact FGF21 with an in-well sensitivity of 1.8 pg/ml. A significant improvement in assay sensitivity was observed in both total FGF21 and active FGF21 QSAs as compared to traditional ELISA. FIG. 20 shows a representative of the standard curve performance for the total and active FGF21 assays. Good standard curve performance was observed.

### Example 7: Optimization of the FGF21 Detection Assays Using Quanterix Simoa

The FGF21 QSAs described in Example 6 was further optimized to improve the sensitivity of the assay.

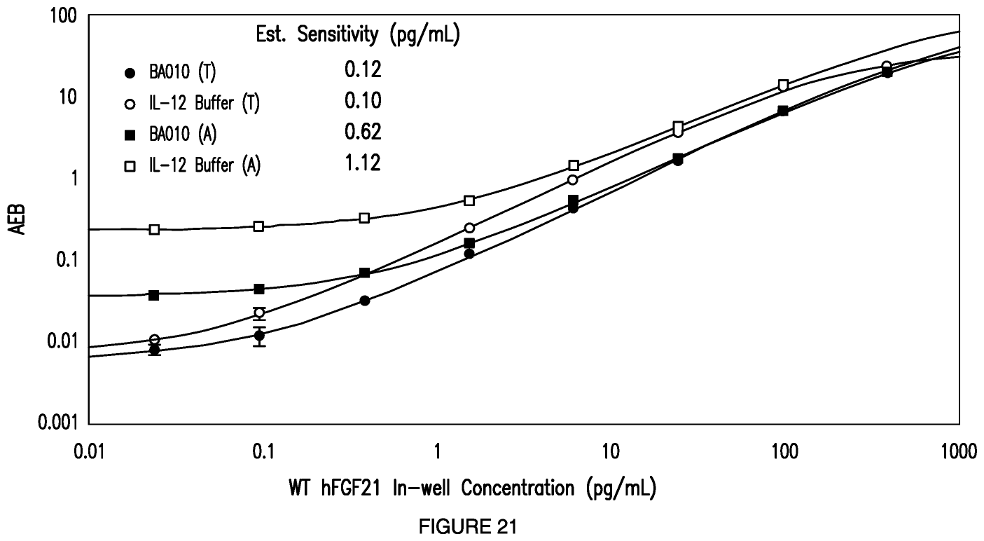

The effect of the type of assay diluent had on the sensitivity of the assays was analyzed. Two different diluents were tested, the BA010 diluent (PBS, 0.5% BSA, 0.25% CHAPS, 5 mM EDTA, 0.35M NaCl, 0.05% Tween-20, 0.05% Proclin 300, pH 7.4) and the IL-12 diluent (PBS, 1.5% BSA, 0.15% Tween-20, 0.05% Proclin 300, pH 7.4). The BA010 diluent worked well for both the total and active FGF21 assays, and resulted in lower background and improved sensitivity (FIG. 21).

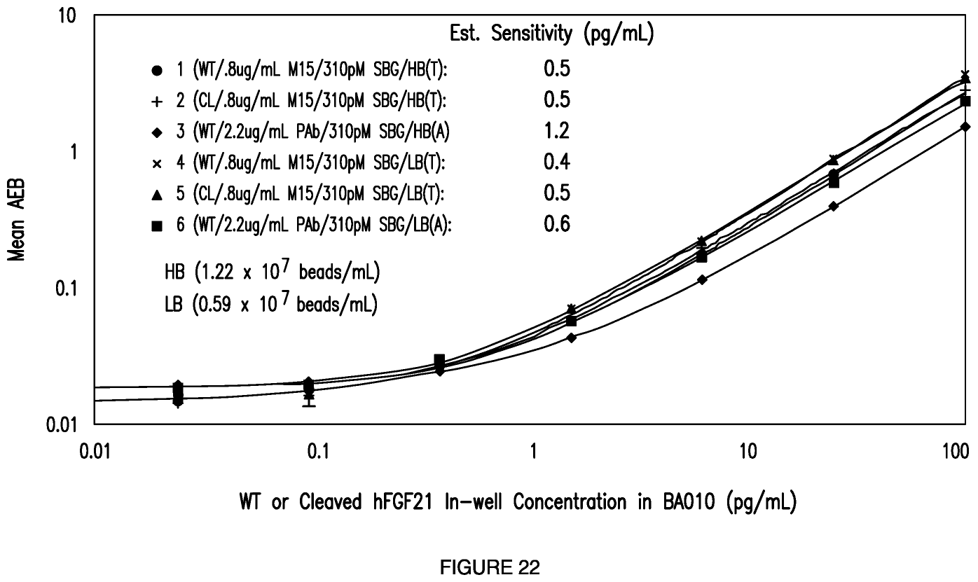

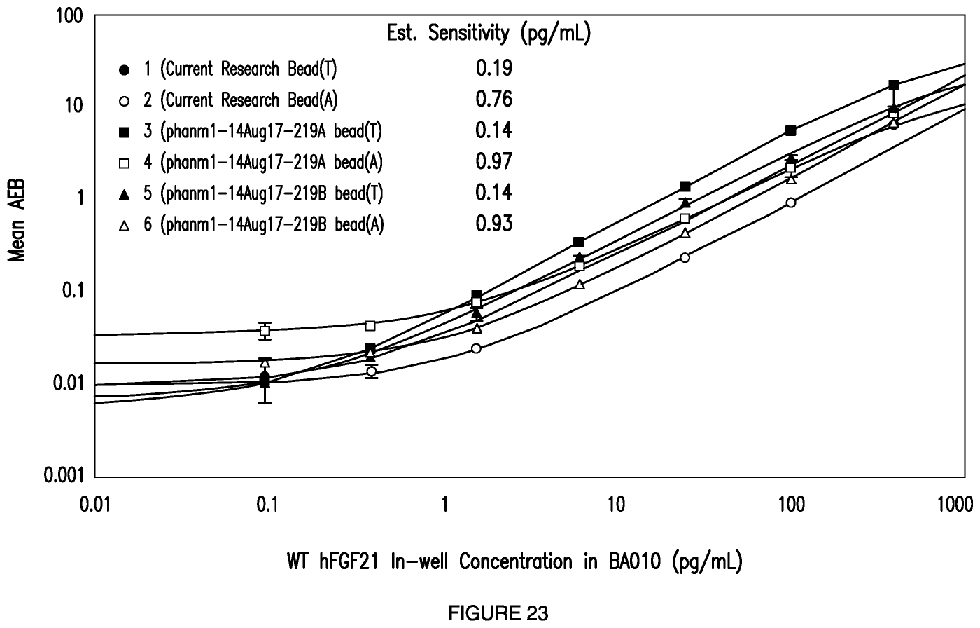

The effect of the concentration of the paramagnetic beads had on the sensitivity of the assay was also analyzed. Two different concentrations were tested, a “high” bead concentration of 1.22×10⁷ beads/ml and a “low” bead concentration of 0.59×10⁷ beads/ml. As shown in FIG. 22, similar assay sensitivity was observed between high bead concentrations and low bead concentrations for the total FGF21 assay. However, improved sensitivity was observed at the low bead concentration for the active FGF21 assay (FIG. 22). In particular, the active FGF21 assay had an in-well sensitivity of 1.2 pg/ml when the high bead concentration was used as compared to the in-well sensitivity of 0.6 pg/ml, which was observed with the low bead concentration. Three different paramagnetic bead lots were also analyzed. As shown in FIG. 23, similar binding curves and assay sensitivity were observed with the current and new lots of capture paramagnetic beads. Optimized assay parameters are shown in Table 14.

TABLE 14
Optimized Assay Parameters.

| Reagent | Concentration |
|---|---|
| Assay Diluent (BA010) | 1X |
| Bead | 0.59 × 10⁷ beads/ml |
| Detector Antibody (Total, Active) | 0.8 μg/mL, 2.2 μg/mL |
| SBG | 310 pM |

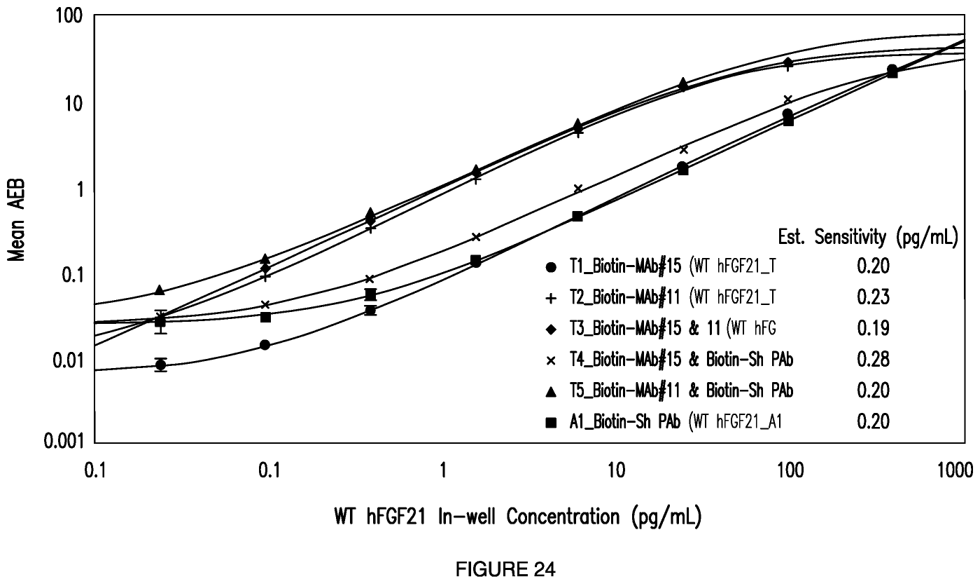

Different detector antibodies were tested for the total FGF21 assay. Antibodies mAb11, mAb15 and C-ter pAb were tested. Similar sensitivities were observed with the various detector antibodies in the total FGF21 assay (FIG. 24). However, the curve for mAb15 had the lowest background.

From the results shown in FIGS. 14, 19, and 22, the optimized concentrations of the detector antibodies and SBG for the total and active FGF21 assays were determined (Table 15). Assay sensitivity for both the total FGF21 and active FGF21 assay were improved when the concentrations of the detector antibody and SBG were increased. The sensitivity of the total FGF21 assay improved with a detector antibody concentration of 0.8 μg/mL and an SBG concentration of 310 pM, and the sensitivity of the activity FGF21 assay improved with a detector antibody concentration of 2.2 μg/mL and an SBG concentration of 310 pM.

TABLE 15
Optimization of Detector Antibody Concentration and SBG.
Total FGF21 Assay Sensitivity (pg/ml)

| Detector Antibody | SBG (pM) | |
|---|---|---|
| (μg/mL) | 310 | 155 |
| 0.8 | 0.1 | 0.4 |
| 0.4 | 0.7 | 0.3 |
| 2.2 | 0.5 | 1.1 |
| 1.1 | 2.2 | 1.4 |

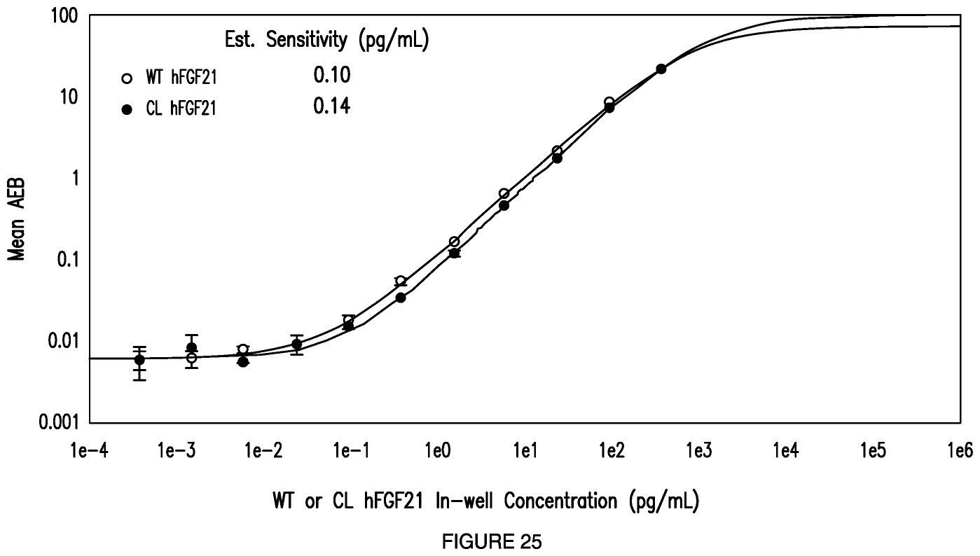

The total FGF21 assay was further analyzed to determine if a hook effect is observed. A hook effect is typically observed when a high amount of analyte is present in a sample and the observed value is falsely lower. The assays were performed as follows: for the total assay, capture using performed by mAb4 conjugated paramagnetic beads at a concentration of 0.59×10⁷ beads/ml and detection was performed using 0.8 μg/mL of biotinylated mAb15; for the active assay, capture was performed using mAb4 conjugated paramagnetic beads at a concentration of 0.59×10⁷ beads/ml and detection was performed using 2.2 μg/mL biotinylated Sheep anti-FGF21 C-term pAb. As shown in FIG. 25, no hook effect was observed with the total FGF21 assay. Further, the total FGF21 assay detected intact human FGF21 and FAP-cleaved human FGF21 (CL hFGF21) with similar sensitivities (FIG. 25).

### Example 8: Analysis of Plasma Samples Using FGF21 QSA

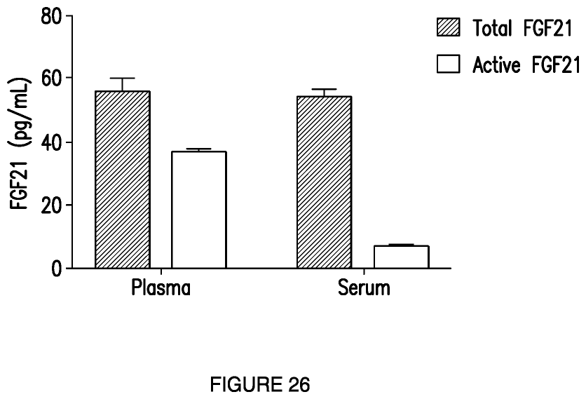

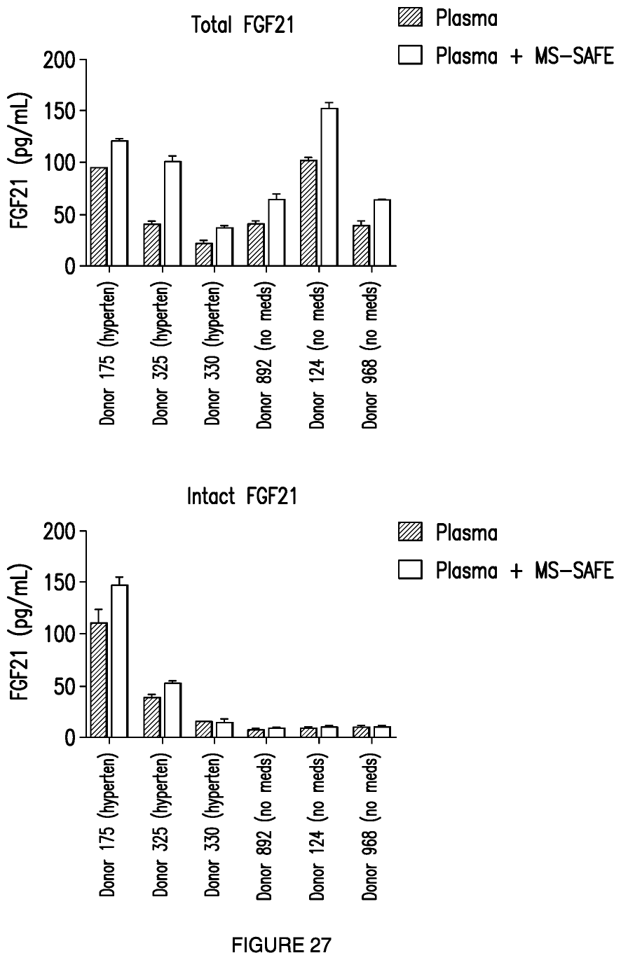

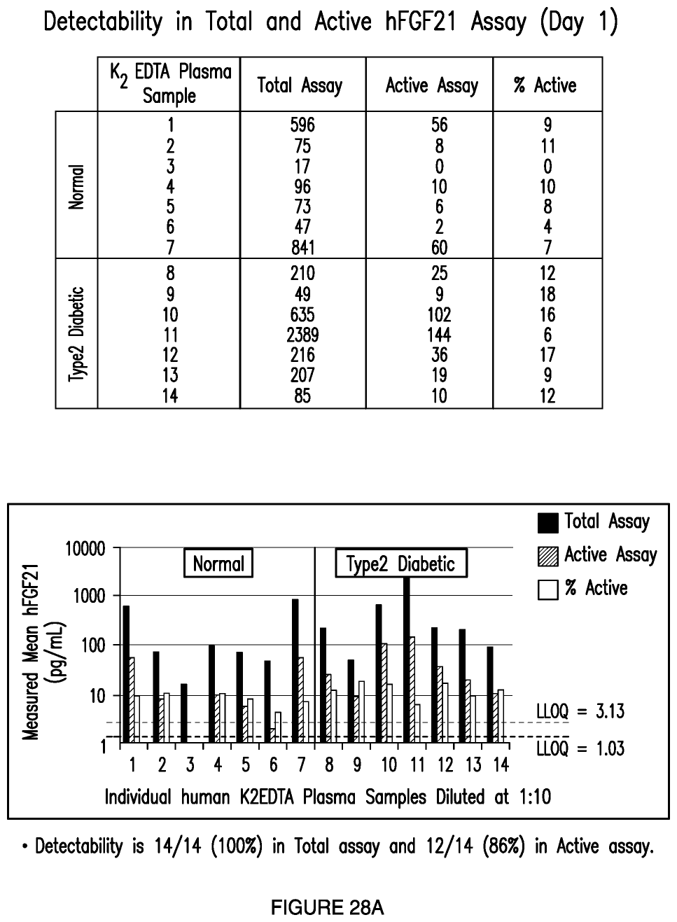

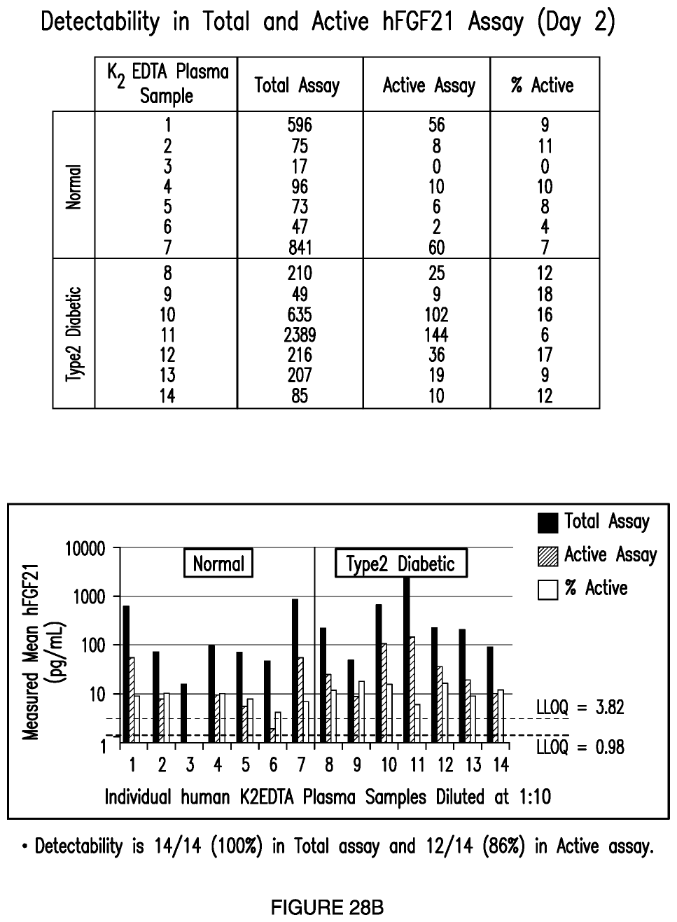

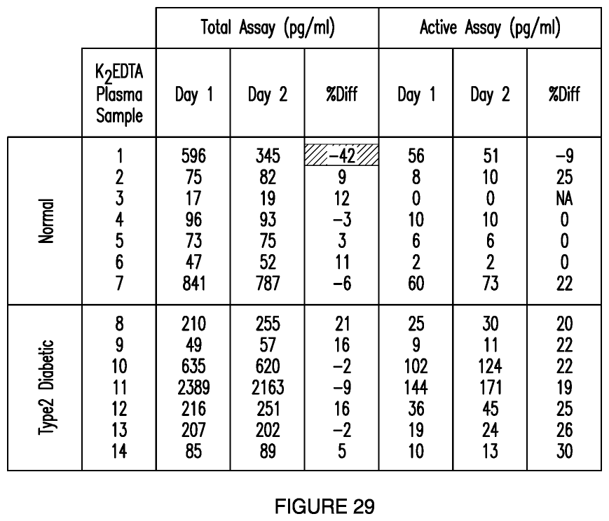

The total and active FGF21 QSAs were used to analyze samples obtained and freshly prepared from a healthy human donor. The assays were performed as described in Example 6. As shown in FIG. 26, the assay was able to detect low levels of active FGF21 in the serum sample of the healthy donor. Additional experiments were performed in donors that were hypertensive or were not on any medications and were compared with the use of MS-SAFE, a protease inhibitor cocktail (FIG. 27). Additional experiments were performed in type 2 diabetes patients. As shown in FIG. 28A-B, out of 14 samples, FGF21 was detected in all samples (100%) using the total FGF21 assay. For the active FGF21 assay, FGF21 protein was detected in 12/14 samples (86%) (FIG. 28A-B). The results obtained from the assays were reproducible (FIG. 29). Reproducibility was acceptable within 30% difference in both total FGF21 and active FGF21 assays.

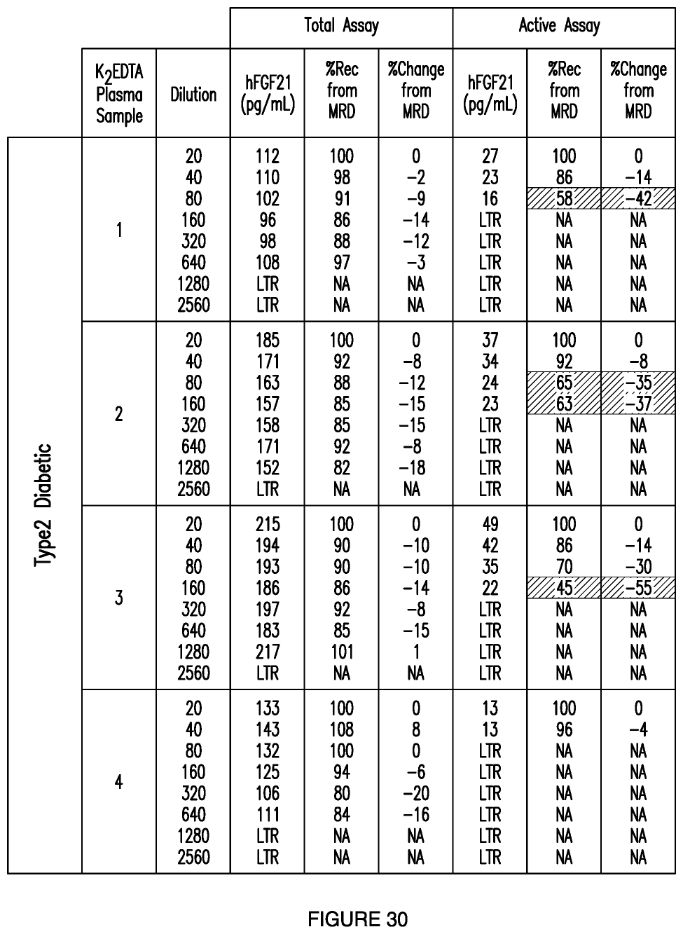

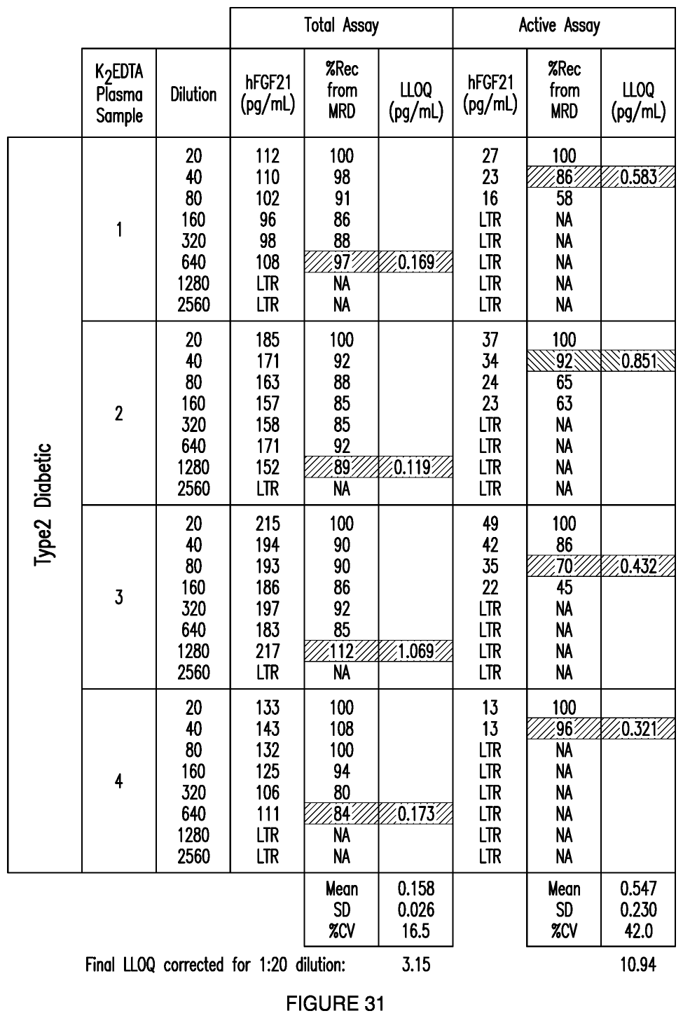

The linearity of dilution was analyzed for the total and active FGF21 assays. A linearity of dilution was acceptable within 30% change from minimum required dilution (MRD) (1:20 dilution) for the total FGF21 and at 1:40 dilution in the active FGF21 assay (FIG. 30). A trend of a higher concentration at the initial MRD was observed. The LLOQ was determined for the total and active FGF21 assays. Preliminary LLOQ was determined to be 3.15 pg/ml and 10.94 pg/ml for the total FGF21 and active FGF21 assays, respectively, based on acceptable recovery within 30% of the mean calculated concentration at the highest dilution factor (FIG. 31).

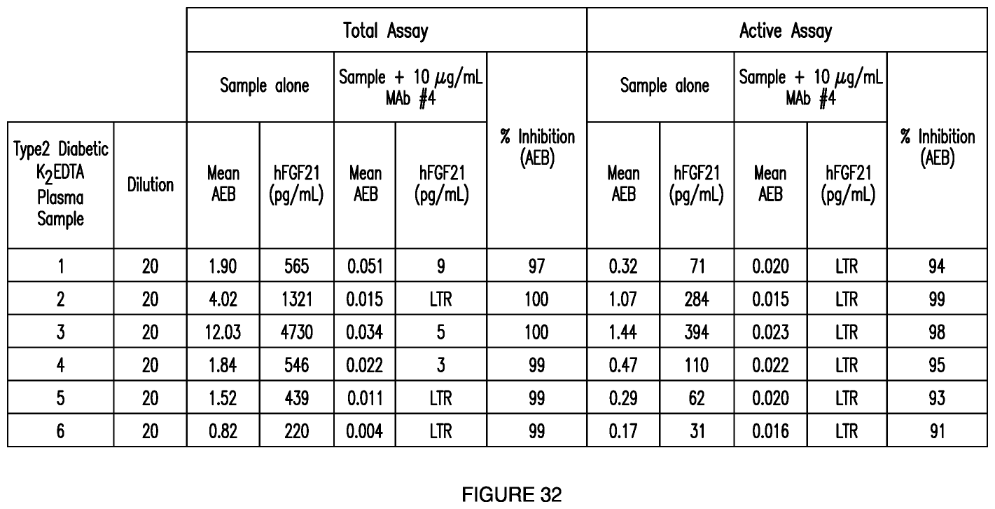

The specificity of the assays was analyzed further. As shown in FIG. 32, specificity was demonstrated by greater than 90% inhibition of AEB values of all six type 2 diabetic plasma sample in the presence of 10 μg/mL of mAb4 in the total FGF21 and active FGF21 assays.

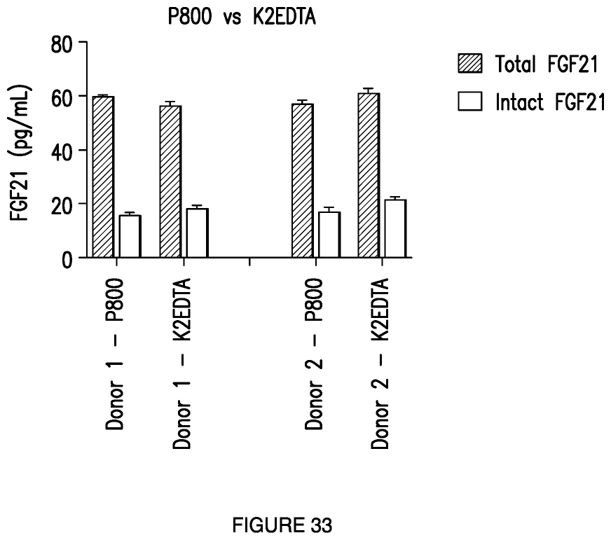

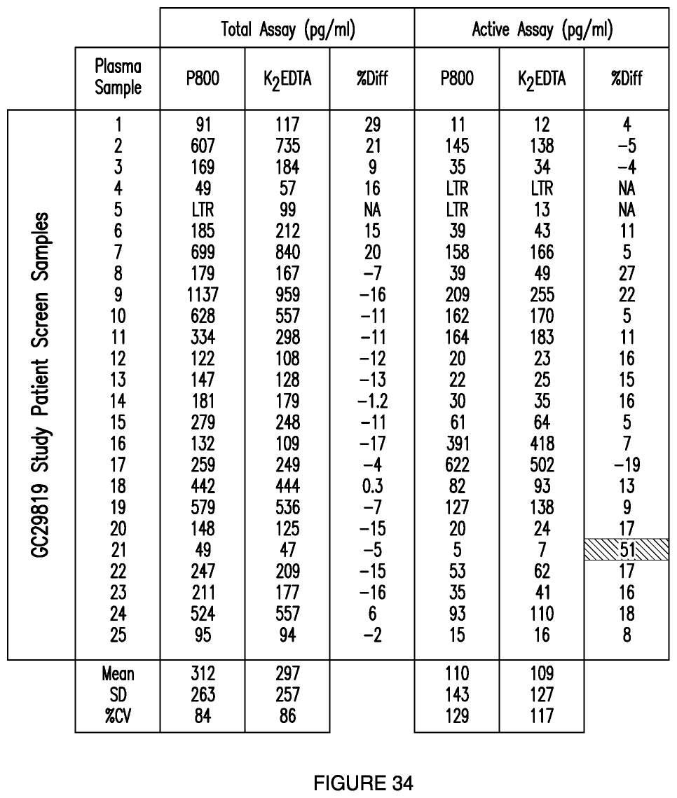

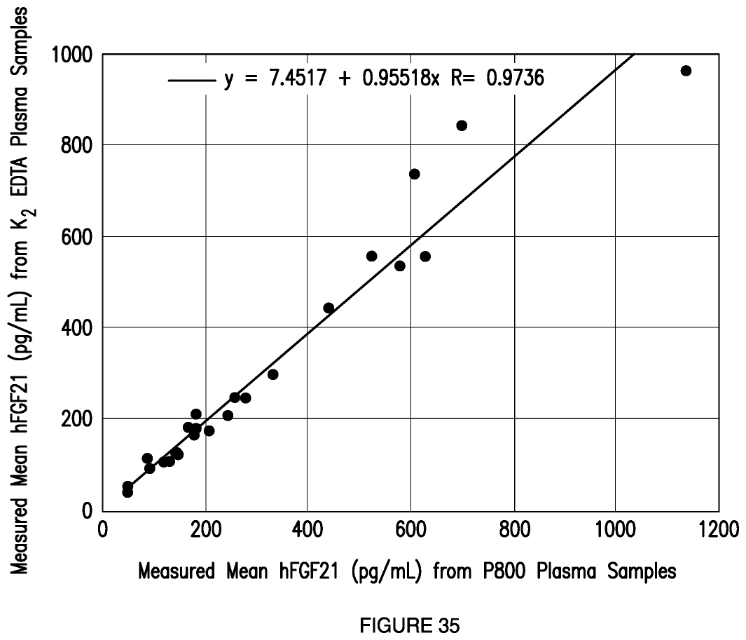

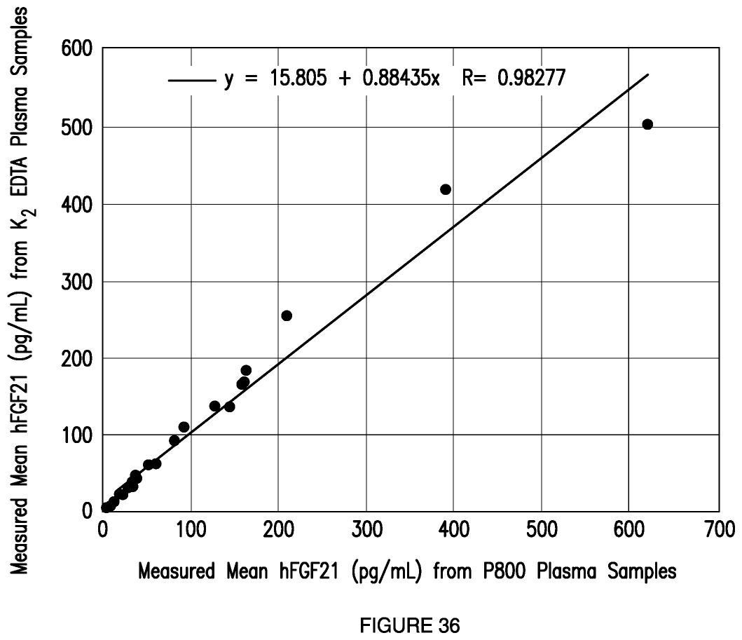

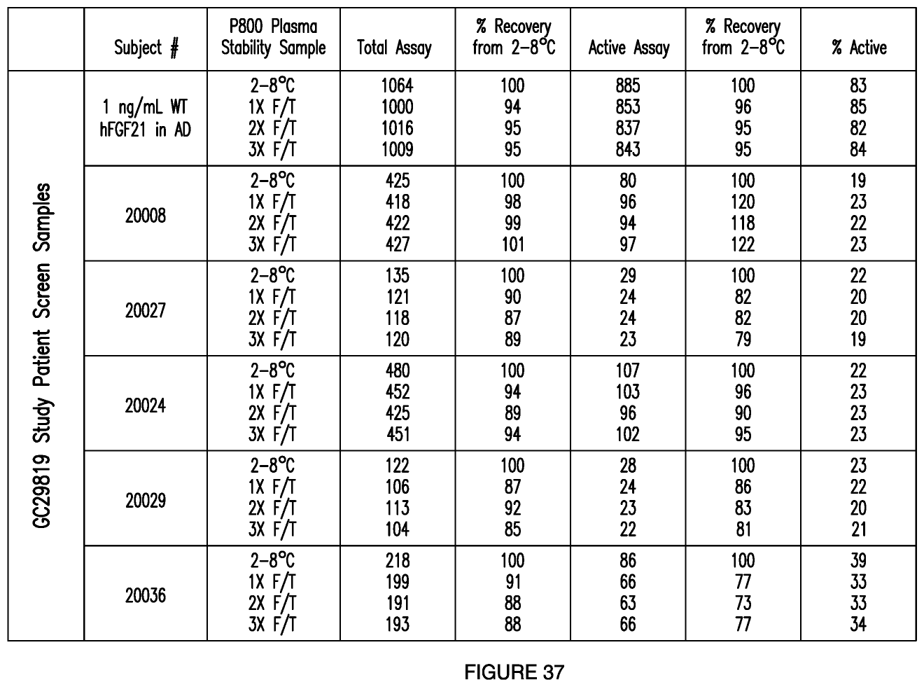

The use of the P800 blood collection system, which includes a combination of protease, esterase and DPP-IV inhibitors and includes the anticoagulant K₂-EDTA, was compared with the use of K₂-EDTA alone (FIG. 33). Comparable results within acceptable ±30% difference between P800 and K2EDTA screen plasma samples were observed in the total FGF21 and active FGF21 assays (FIG. 34). A good correlation between P800 and K₂-EDTA screen plasma samples were observed in the total FGF21 and active FGF21 assays (FIGS. 35-36). The stability of the plasma samples after being stored at 2-8° C. were analyzed. As shown in FIG. 37, sample stability within acceptable 30% recovery from 2-8° C. stability sample was observed in total FGF21 and active FGF21 assays.

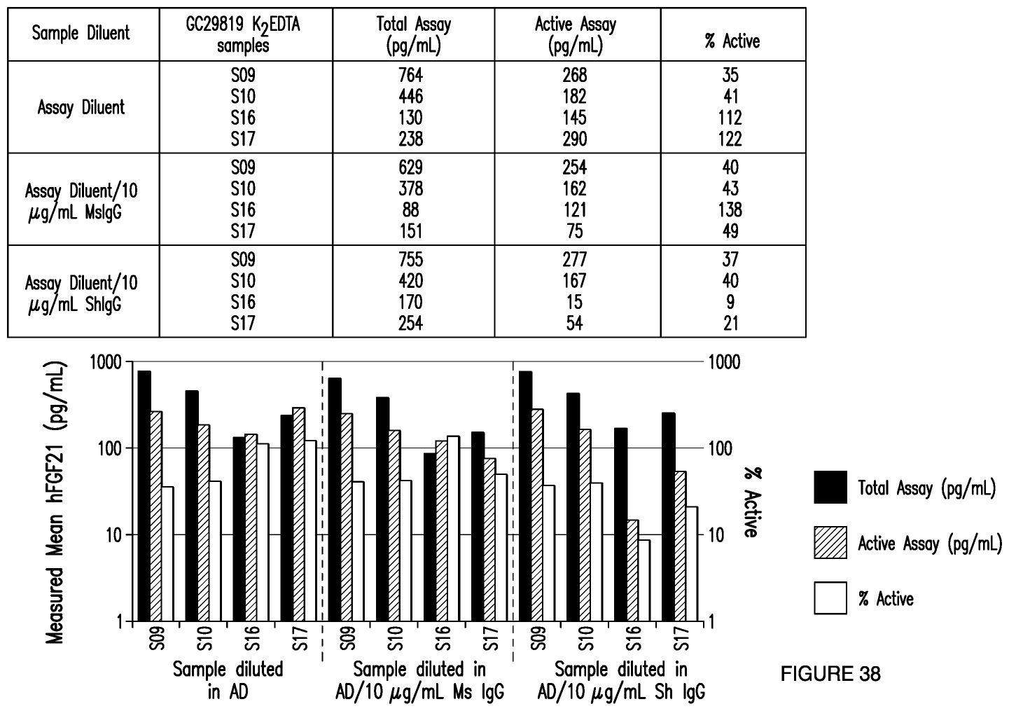

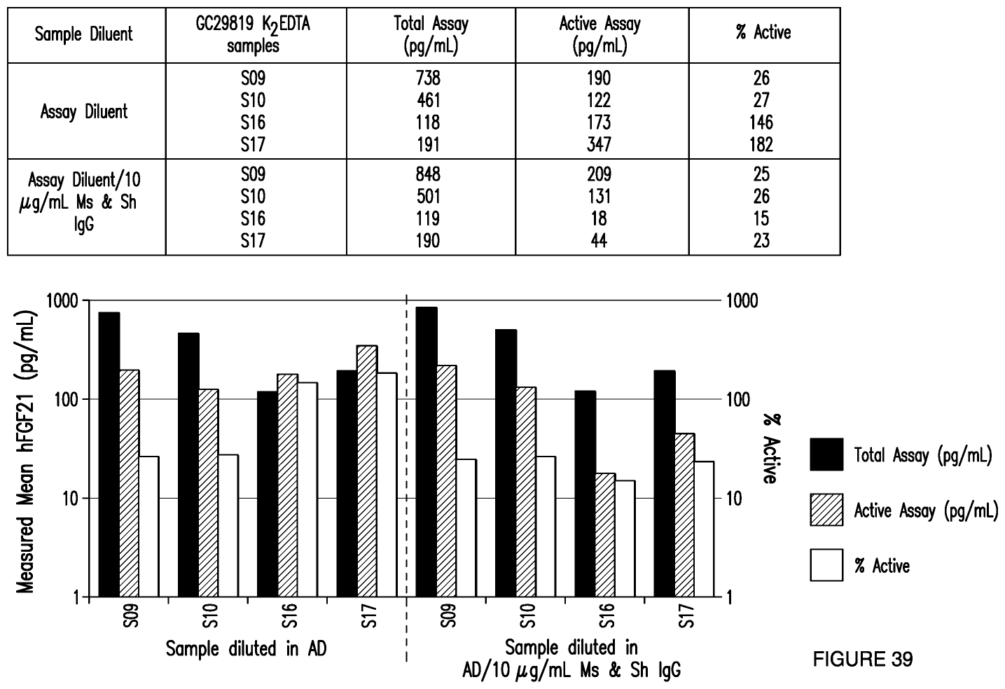

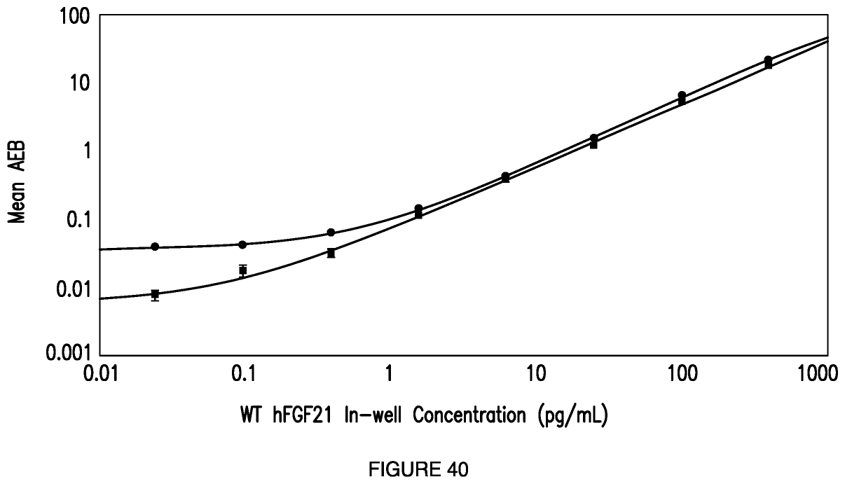

As shown in FIGS. 38 and 39, a higher than 100% active ratio was observed in K₂-EDTA screen plasma samples that were analyzed by the total and active FGF21 assays suggesting interference by heterophilic antibodies. In particular, a higher than 100% active ratio was observed from K₂-EDTA screen plasma samples 16 and 17 but not 9 and 10 from the GC29819 study when assay diluent was used alone. Samples 16 and 17, which had an active ratio higher than 100%, contained human anti-mouse antibodies (HAMA) and human anti-sheep antibodies (HASA), indicating that the presence of HAMA and HASA in patient plasma samples interfered with the accuracy of the total and active assays. As shown in FIGS. 38 and 39, HAMA affected both the total and active assay; whereas, HASA affected only the active assay. The addition of 10 μg/ml of mouse IgG to the diluent of the total assay and the addition of 10 μg/ml of sheep IgG in the diluent of the active assay effectively removed HAMA and HASA interference, respectively, and resolved the higher than 100% active ratio that was observed (FIGS. 38 and 39). As shown in FIG. 40, the presence of 10 μg/ml of anti-mouse IgG or anti-sheep IgG in the assay diluent did not affect the standard curve of the total and active assays, respectively.

### Example 9: Chimeric Anti-FGF21 Antibodies

Antibodies mAb4, mAb9, mAb11 and mAb15 were grafted onto human IgG1 frameworks with a K149C mutation to generate mouse/human chimeric anti-FGF21 antibodies that have the mouse VH and VL regions and the human constant region with the K149C mutation. Amino acid sequences for the chimeric antibodies are provided below in Tables 16-19 and FIGS. 41A and 41B.

TABLE 16
Chimeric Ab4 (FGF21.GN36.4.hIgG1; PRO418189)

Full-length Light Chain Amino Acid Sequence
QIVLTQSPAIMSAPLGERVTMTCTASSSVSSSYLHWYQQKPGSSPKVWIY
RTTNLASGVPTRFSGSGSGTSYSLTISSMEAEDAATYYCHQYHRSPPTWT
FGGGTKVEIKRTVAAPSVFIFPPSDEQLKSGTASVVCLLNNFYPREAKVQ
WCVDNALQSGNSQESVTEQDSKDSTYSLSSTLTLSKADYEKHKVYACEVT
HQGLSSPVTKSFNRGEC (SEQ ID NO: 63)
Full-length Heavy Chain Amino Acid Sequence
EVKLVESGGDLVKPGGSLKLSCAASGFTFSSYGMSWVRQTPDKRLEWVAT
ISTGGGYTYYPDSVKGRFTISRDNAKNTLYLQMSSLRSEDTAMYYCARHD
LVDWYFDVWGTGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
(SEQ ID NO: 67)
Light Chain Variable Region
QIVLTQSPAIMSAPLGERVTMTCTASSSVSSSYLHWYQQKPGSSPKVWIY
RTTNLASGVPTRFSGSGSGTSYSLTISSMEAEDAATYYCHQYHRSPPTWT
FGGGTKVEIK (SEQ ID NO: 71)
Heavy Chain Variable Region
EVKLVESGGDLVKPGGSLKLSCAASGFTFSSYGMSWVRQTPDKRLEWVAT
ISTGGGYTYYPDSVKGRFTISRDNAKNTLYLQMSSLRSEDTAMYYCARHD
LVDWYFDVWGTGTTVTVSS (SEQ ID NO: 75)

TABLE 17
Chimeric Ab9 (FGF21.GN36.9.hIgG1; PRO418190)

Full-length Light Chain Amino Acid Sequence
DIQMTQSPASLSASVGETVIITCRASENIYSYLAWYQQKQGKSPQLLVY
NIRTLAEGVPSRFSGSGSGTQFSLKINSLQPEDFGSYYCQHHYDSPWTF
GGGTKVEIKRTVAAPSVFIFPPSDEQLKSGTASVVCLLNNFYPREAKVQ
WCVDNALQSGNSQESVTEQDSKDSTYSLSSTLTLSKADYEKHKVYACEV
THQGLSSPVTKSFNRGEC (SEQ ID NO: 62)
Full-length Heavy Chain Amino Acid Sequence
EVQLQQSGPELVKPGASVKIPCKASGYTFTDYYMGWVKQSHGKSLEWIG
DINPNNGVTINNQNFKGKATLTVDKSSSTAYMELRSLASEDTAVYYCTR
GYGGALDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGK (SEQ ID NO: 66)
Light Chain Variable Region
DIQMTQSPASLSASVGETVIITCRASENIYSYLAWYQQKQGKSPQLLVY
NIRTLAEGVPSRFSGSGSGTQFSLKINSLQPEDFGSYYCQHHYDSPWTF
GGGTKVEIK (SEQ ID NO: 70)
Heavy Chain Variable Region
EVQLQQSGPELVKPGASVKIPCKASGYTFTDYYMGWVKQSHGKSLEWIG
DINPNNGVTINNQNFKGKATLTVDKSSSTAYMELRSLASEDTAVYYCTR
GYGGALDYWGQGTSVTVSS (SEQ ID NO: 74)

TABLE 18
Chimeric Ab11 (FGF21.GN36.11.hIgG1; PRO418191)

Full-length Light Chain Amino Acid Sequence
QIVLTQSPALMSASPGERVTMTCSAGSSVSYMYWYQQKPRSSPKPWIYLT
SNLASGVPARFSGSGSGTSYSLTISSMEAEDAATYYCQQWSSNPRTFGGG
TKVEIKRTVAAPSVFIFPPSDEQLKSGTASVVCLLNNFYPREAKVQWCVD
NALQSGNSQESVTEQDSKDSTYSLSSTLTLSKADYEKHKVYACEVTHQGL
SSPVTKSFNRGEC (SEQ ID NO: 61)
Full-length Heavy Chain Amino Acid Sequence
QVQLQQSGAELARPGASVKLSCKASGYTFTNYGISWVKQRTGQGLEWIGE
IYPRSDNTYYNEKFKGKATLTADKSSSTAYMELRSLTSEDSAVYFCTRSD
YGFFDYWGQGTTLTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
(SEQ ID NO: 65)
Light Chain Variable Region
ALMSASPGERVTMTCSAGSSVSYMYWYQQKPRSSPKPWIYLTSNLASGVP
ARFSGSGSGTSYSLTISSMEAEDAATYYCQQWSSNPRTFGGGTKVEIK
(SEQ ID NO: 69)
Heavy Chain Variable Region
QVQLQQSGAELARPGASVKLSCKASGYTFTNYGISWVKQRTGQGLEWIGE
IYPRSDNTYYNEKFKGKATLTADKSSSTAYMELRSLTSEDSAVYFCTRSD
YGFFDYWGQGTTLTVSS (SEQ ID NO: 73)

TABLE 19
Chimeric Ab15 (FGF21.GN36.15.hIgGl; PR0418192)

Full-length Light Chain Amino Acid Sequence
DVLMTQTPLSLPVSLGDQASISCRSSQIIVHNNGDTYLEWYLQKPGQSPK
LLIYKISNRFSGVPDRFSGSGSGTDFTLKISRVEAEDLGVYYCFQGSHVP
YTFGGGTKVEIKRTVAAPSVFIFPPSDEQLKSGTASVVCLLNNFYPREAK
VQWCVDNALQSGNSQESVTEQDSKDSTYSLSSTLTLSKADYEKHKVYACE
VTHQGLSSPVTKSFNRGEC (SEQ ID NO: 60)
Full-length Heavy Chain Amino Acid Sequence
QVQLIQSGPGLVQPSQSLSITCTVSGFSLTGYAIHWVRQSPGKGLEWLGM
IWKSGNTDYNAAFMSRLSITKDNSKSQVFFKMNSLQADDTAIYYCARNGY
DYEFVYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
(SEQ ID NO: 64)
Light Chain Variable Region
DVLMTQTPLSLPVSLGDQASISCRSSQIIVHNNGDTYLEWYLQKPGQSPK
LLIYKISNRFSGVPDRFSGSGSGTDFTLKISRVEAEDLGVYYCFQGSHVP
YTFGGGTKVEIK (SEQ ID NO: 68)
Heavy Chain Variable Region
QVQLIQSGPGLVQPSQSLSITCTVSGFSLTGYAIHWVRQSPGKGLEWLGM
IWKSGNTDYNAAFMSRLSITKDNSKSQVFFKMNSLQADDTAIYYCARNGY
DYEFVYWGQGTLVTVSS (SEQ ID NO: 72)

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

What is claimed is:

1. An immunoassay method for determining the amount of total FGF21 protein in a sample comprising:
(a) contacting a capture antibody that binds to an epitope present within amino acid residues 5-172 of FGF21 with the sample to generate a sample-capture antibody combination material;
(b) contacting the sample-capture antibody combination material with a detector antibody that binds to an epitope present within amino acid residues 5-172 of FGF21;
(c) detecting the detector antibody bound to the sample-capture antibody combination material; and
(d) calculating an amount of total FGF21 protein present in the sample based on the level of the detector antibody bound.

2. The immunoassay method of claim 1, wherein the capture antibody and the detector antibody bind to different epitopes within amino acid residues 5-172 of FGF21.

3. An immunoassay method for determining the amount of active FGF21 protein in a sample comprising:
(a) contacting a capture antibody that binds to an epitope present within amino acid residues 5-172 of FGF21 with the sample to generate a sample-capture antibody combination material;
(b) contacting the sample-capture antibody combination material with a detector antibody that binds to an epitope present within amino acid residues 173-182 of FGF21;
(c) detecting the detector antibody bound to the sample-capture antibody combination material; and
(d) calculating an amount of active FGF21 protein present in the sample based on the level of the detector antibody bound.

4. An immunoassay method for determining the ratio of active FGF21 protein to total FGF21 protein in a sample comprising:
(a) (i) contacting a first capture antibody that binds to an epitope present within amino acid residues 5-172 of FGF21 with the sample to generate a first sample-capture antibody combination material; (ii) contacting the first sample-capture antibody combination material with a first detector antibody that binds to an epitope present within amino acid residues 5-172 of FGF21; (iii) detecting the first detector antibody bound to the sample-capture antibody combination material; and (iv) calculating an amount of total FGF21 protein present in the sample based on the level of the first detector antibody bound;
(b) (i) contacting a second capture antibody that binds to an epitope present within amino acid residues 5-172 of FGF21 with the sample to generate a second sample-capture antibody combination material; (ii) contacting the second sample-capture antibody combination material with a second detector antibody that binds to an epitope present within amino acid residues 173-182 of FGF21; (iii) detecting the second detector antibody bound to the sample-capture antibody combination material; and (iv) calculating an amount of active FGF21 protein present in the sample based on the level of the second detector antibody bound; and
(c) comparing the amount of total FGF21 protein as determined by step (a) with the amount of active FGF21 protein as determined by step (b) to determine the ratio of active FGF21 protein to total FGF21 protein in the sample.

5. The immunoassay method of claim 4, wherein the first capture antibody and the first detector antibody bind to different epitopes within amino acid residues 5-172 of FGF21.

6. The immunoassay method of claim 4, wherein the first capture antibody and second capture antibody are the same antibody.

7. The immunoassay method of any one of claims 1-6, wherein the immunoassay is an enzyme-linked immunosorbent assay (ELISA).

8. The immunoassay method of any one of claims 1-7, wherein one or more of the capture antibody, first capture antibody and second capture antibody is immobilized to a paramagnetic bead.

9. The immunoassay method of any one of claims 1-8, wherein one or more of the detector antibody, first detector antibody and second detector antibody is conjugated to biotin.

10. The immunoassay method of any one of claims 1-9, wherein one or more of the capture antibody, first capture antibody and second capture antibody binds to FGF21 with a K_{d} from about 10⁻¹⁰ M to 10⁻¹³ M.

11. The immunoassay method of any one of claims 1 and 4-9, wherein one or more of the detector antibody and first detector antibody binds to FGF21 with a K_{d} from about 10⁻¹⁰ M to 10⁻¹³ M.

12. The immunoassay method of any one of claims 1-11, wherein the sample is a blood sample.

13. The immunoassay method of any one of claims 1-11, wherein the sample is a plasma sample.

14. The immunoassay method of any one of claims 1-13, wherein the method detects the amount of total or active FGF21 protein in the sample at an in-well sensitivity from about 2 pg/ml to about 20 pg/ml.

15. The immunoassay method of any one of claims 1-13, wherein the immunoassay method is performed using a single molecule detection instrument.

16. The immunoassay method of claim 15, wherein the single molecule detection instrument is the Quanterix Simoa HD-1 Analyzer™.

17. The immunoassay method of claim 15 or 16, wherein the method detects the amount of total or active FGF21 protein in the sample at an in-well sensitivity from about 0.2 pg/ml to about 0.5 pg/ml.

18. The immunoassay method of any one of claims 1-17, wherein one or more of the capture antibody, first capture antibody and second capture antibody comprises:
(a) a heavy chain variable region CDR1 comprising an amino acid sequence selected from the group consisting of SEQ ID NOs: 26 and 27, and conservative substitutions thereof;
(b) a heavy chain variable region CDR2 domain comprising an amino acid sequence selected from the group consisting of SEQ ID NOs: 30 and 31, and conservative substitutions thereof;
(c) a heavy chain variable region CDR3 domain comprising an amino acid sequence selected from the group consisting of SEQ ID NOs: 34 and 35, and conservative substitutions thereof;
(d) a light chain variable region CDR1 domain comprising an amino acid sequence selected from the group consisting of SEQ ID NOs: 38 and 39, and conservative substitutions thereof;
(e) a light chain variable region CDR2 domain comprising an amino acid sequence selected from the group consisting of SEQ ID NOs: 42 and 43, and conservative substitutions thereof; and
(f) a light chain variable region CDR3 domain comprising an amino acid sequence selected from the group consisting of SEQ ID NOs: 46 and 47, and conservative substitutions thereof.

19. The immunoassay of any one of claims 1-17, wherein one or more of the capture antibody, first capture antibody and second capture antibody comprises:
(a) a heavy chain variable region comprising an amino acid sequence selected from the group consisting of SEQ ID NOs: 54, 55, 74 and 75, and conservative substitutions thereof; and
(b) a light chain variable region comprising an amino acid sequence selected from the group consisting of SEQ ID NOs: 50, 51, 70 and 71, and conservative substitutions thereof.

20. The immunoassay of any one of claims 1-17, wherein one or more of the capture antibody, first capture antibody and second capture antibody comprises:
(a) a heavy chain comprising an amino acid sequence selected from the group consisting of SEQ ID NOs: 22, 23, 66 and 67, and conservative substitutions thereof; and
(b) a light chain comprising an amino acid sequence selected from the group consisting of SEQ ID NOs: 18, 19, 62 and 63, and conservative substitutions thereof.

21. The immunoassay method of any one of claims 1 and 4-17, wherein one or more of the detector antibody and first detector antibody comprises:
(a) a heavy chain variable region CDR1 comprising an amino acid sequence selected from the group consisting of SEQ ID NOs: 28 and 29, and conservative substitutions thereof;
(b) a heavy chain variable region CDR2 domain comprising an amino acid sequence selected from the group consisting of SEQ ID NOs: 32 and 33, and conservative substitutions thereof;
(c) a heavy chain variable region CDR3 domain comprising an amino acid sequence selected from the group consisting of SEQ ID NOs: 36 and 37, and conservative substitutions thereof;
(d) a light chain variable region CDR1 domain comprising an amino acid sequence selected from the group consisting of SEQ ID NOs: 40 and 41, and conservative substitutions thereof;
(e) a light chain variable region CDR2 domain comprising an amino acid sequence selected from the group consisting of SEQ ID NOs: 44 and 45, and conservative substitutions thereof; and
(f) a light chain variable region CDR3 domain comprising an amino acid sequence selected from the group consisting of SEQ ID NOs: 48 and 49, and conservative substitutions thereof.

22. The immunoassay of any one of claims 1 and 4-17, wherein one or more of the detector antibody and first detector antibody comprises:
(a) a heavy chain variable region comprising an amino acid sequence selected from the group consisting of SEQ ID NOs: 56, 57, 72 and 73, and conservative substitutions thereof; and
(b) a light chain variable region comprising an amino acid sequence selected from the group consisting of SEQ ID NOs: 52, 53, 68 and 69, and conservative substitutions thereof.

23. The immunoassay of any one of claims 1 and 4-17, wherein one or more of the detector antibody and first detector antibody comprises:
(a) a heavy chain comprising an amino acid sequence selected from the group consisting of SEQ ID NOs: 24, 25, 64 and 65, and conservative substitutions thereof; and
(b) a light chain comprising an amino acid sequence selected from the group consisting of SEQ ID NOs: 20, 21, 60 and 61, and conservative substitutions thereof.

24. The immunoassay method of claim 18, wherein one or more of the capture antibody, first capture antibody and second capture antibody comprises:
(a) a heavy chain variable region CDR1 comprising the amino acid sequence of SEQ ID NO: 26, and conservative substitutions thereof;
(b) a heavy chain variable region CDR2 domain comprising the amino acid sequence of SEQ ID NO: 30, and conservative substitutions thereof;
(c) a heavy chain variable region CDR3 domain comprising the amino acid sequence of SEQ ID NO: 34, and conservative substitutions thereof;
(d) a light chain variable region CDR1 domain comprising the amino acid sequence of SEQ ID NO: 38, and conservative substitutions thereof;
(e) a light chain variable region CDR2 domain comprising the amino acid sequence of SEQ ID NO: 42, and conservative substitutions thereof; and
(f) a light chain variable region CDR3 domain comprising the amino acid sequence of SEQ ID NO: 46, and conservative substitutions thereof.

25. The immunoassay of claim 24, wherein one or more of the capture antibody, first capture antibody and second capture antibody comprises:
(a) a heavy chain variable region comprising the amino acid sequence of SEQ ID NO: 54, and conservative substitutions thereof; and
(b) a light chain variable region comprising the amino acid sequence of SEQ ID NO: 50, and conservative substitutions thereof.

26. The immunoassay of claim 25, wherein one or more of the capture antibody, first capture antibody and second capture antibody comprises:
(a) a heavy chain comprising the amino acid sequence of SEQ ID NO: 22, and conservative substitutions thereof; and
(b) a light chain comprising the amino acid sequence of SEQ ID NO: 18, and conservative substitutions thereof.

27. The immunoassay method of claim 21, wherein one or more of the detector antibody and first detector antibody comprises:
(a) a heavy chain variable region CDR1 comprising the amino acid sequence of SEQ ID NO: 29, and conservative substitutions thereof;
(b) a heavy chain variable region CDR2 domain comprising the amino acid sequence of SEQ ID NO: 33, and conservative substitutions thereof;
(c) a heavy chain variable region CDR3 domain comprising the amino acid sequence of SEQ ID NO: 37, and conservative substitutions thereof;
(d) a light chain variable region CDR1 domain comprising the amino acid sequence of SEQ ID NO: 41, and conservative substitutions thereof;
(e) a light chain variable region CDR2 domain comprising the amino acid sequence of SEQ ID NO: 45, and conservative substitutions thereof; and
(f) a light chain variable region CDR3 domain comprising the amino acid sequence of SEQ ID NO: 49, and conservative substitutions thereof.

28. The immunoassay of claim 27, wherein one or more of the detector antibody and first detector antibody comprises:
(a) a heavy chain variable region comprising the amino acid sequence of SEQ ID NO: 57, and conservative substitutions thereof; and
(b) a light chain variable region comprising the amino acid sequence of SEQ ID NO: 53, and conservative substitutions thereof.

29. The immunoassay of claim 28, wherein one or more of the detector antibody and first detector antibody comprises:
(a) a heavy chain comprising the amino acid sequence of SEQ ID NO: 25, and conservative substitutions thereof; and
(b) a light chain comprising the amino acid sequence of SEQ ID NO: 21, and conservative substitutions thereof.

30. The immunoassay method of any one of claims 1-17, wherein one or more of the capture antibody, first capture antibody and second capture antibody competitively binds with an antibody comprising:
(a) a heavy chain variable region CDR1 comprising an amino acid sequence selected from the group consisting of SEQ ID NOs: 26 and 27, and conservative substitutions thereof;
(b) a heavy chain variable region CDR2 domain comprising an amino acid sequence selected from the group consisting of SEQ ID NOs: 30 and 31, and conservative substitutions thereof;
(c) a heavy chain variable region CDR3 domain comprising an amino acid sequence selected from the group consisting of SEQ ID NOs: 34 and 35, and conservative substitutions thereof;
(d) a light chain variable region CDR1 domain comprising an amino acid sequence selected from the group consisting of SEQ ID NOs: 38 and 39, and conservative substitutions thereof;
(e) a light chain variable region CDR2 domain comprising an amino acid sequence selected from the group consisting of SEQ ID NOs: 42 and 43, and conservative substitutions thereof; and
(f) a light chain variable region CDR3 domain comprising an amino acid sequence selected from the group consisting of SEQ ID NOs: 46 and 47, and conservative substitutions thereof.

31. The immunoassay method of any one of claims 1 and 4-17, wherein one or more of the detector antibody and first detector antibody competitively binds with an antibody comprising:
(a) a heavy chain variable region CDR1 comprising an amino acid sequence selected from the group consisting of SEQ ID NOs: 28 and 29, and conservative substitutions thereof;
(b) a heavy chain variable region CDR2 domain comprising an amino acid sequence selected from the group consisting of SEQ ID NOs: 32 and 33, and conservative substitutions thereof;
(c) a heavy chain variable region CDR3 domain comprising an amino acid sequence selected from the group consisting of SEQ ID NOs: 36 and 37, and conservative substitutions thereof;
(d) a light chain variable region CDR1 domain comprising an amino acid sequence selected from the group consisting of SEQ ID NOs: 40 and 41, and conservative substitutions thereof;
(e) a light chain variable region CDR2 domain comprising an amino acid sequence selected from the group consisting of SEQ ID NOs: 44 and 45, and conservative substitutions thereof; and
(f) a light chain variable region CDR3 domain comprising an amino acid sequence selected from the group consisting of SEQ ID NOs: 48 and 49, and conservative substitutions thereof.

32. A kit for detecting total FGF21 protein in a sample comprising:
(a) a capture antibody, or an antigen-binding portion thereof, that binds to an epitope present within amino acid residues 5-172 of FGF21;
(b) a detector antibody, or an antigen-binding portion thereof, that binds to an epitope present within amino acid residues 5-172 of FGF21; and
(c) a detection agent.

33. The kit of claim 32, wherein the capture antibody and the detector antibody bind to different epitopes within amino acid residues 5-172 of FGF21.

34. A kit for detecting active FGF21 protein in a sample comprising:
(a) a capture antibody, or an antigen-binding portion thereof, that binds to an epitope present within amino acid residues 5-172 of FGF21;
(b) a detector antibody, or an antigen-binding portion thereof, that binds to an epitope present within amino acid residues 173-182 of FGF21; and
(c) a detection agent.

35. A kit for determining the ratio of active FGF21 protein to total FGF21 protein in a sample comprising:
(a) (i) a first capture antibody or an antigen-binding portion thereof, that binds to an epitope present within amino acid residues 5-172 of FGF21 and (ii) a first detector antibody, or an antigen-binding portion thereof, that binds to an epitope present within amino acid residues 5-172 of FGF21;
(b) (i) a second capture antibody, or an antigen-binding portion thereof, that binds to an epitope present within amino acid residues 5-172 of FGF21 and (ii) a second detector antibody, or an antigen-binding portion thereof, that binds to an epitope present within amino acid residues 173-182 of FGF21; and
(c) one or more detection agents.

36. The kit of claim 35, wherein the first capture antibody and second capture antibody are the same antibody.

37. The kit of claim 35, wherein the first capture antibody and the first detector antibody bind to different epitopes within amino acid residues 5-172 of FGF21.

38. The kit of any one of claims 32-37, wherein one or more of the capture antibody, first capture antibody and second capture antibody is immobilized to a paramagnetic bead.

39. The kit of any one of claims 32-38, wherein one or more of the detector antibody, first detector antibody and second detector antibody is conjugated to biotin.

40. The kit of any one of claims 32-39, wherein the detection agent is selected from the group consisting of a streptavidin-β-D-galactopyranose conjugate, a streptavidin-horseradish peroxidase conjugate and a combination thereof.

41. The kit of claim 40 further comprising resorufin β-D-galactopyranoside, tetramethylbenzidine, hydrogen peroxide or combinations thereof.

42. The kit of any one of claims 32-41, wherein one or more of the capture antibody, first capture antibody and second capture antibody binds to FGF21 with a K_{d} from about 10⁻¹⁰ M to 10⁻¹³ M.

43. The kit of any one of claims 32 and 35-42, wherein one or more of the detector antibody and first detector antibody binds to FGF21 with a K_{d} from about 10⁻¹⁰ M to 10⁻¹³ M.

44. The kit of any one of claims 32 and 35-43, wherein the detector antibody or first detector antibody has a concentration from about 0.1 μg/ml to about 1 μg/ml.

45. The kit of any one of claims 33-42, wherein one or more of the detector antibody or second detector antibody has a concentration from about 1 μg/ml to about 3 μg/ml.

46. The kit of claim 40, wherein the streptavidin-β-D-galactopyranose conjugate has a concentration from about 100 pM to about 400 pM.

47. The kit of any one of claims 32-46, wherein one or more of the capture antibody, first capture antibody and second capture antibody comprises:
(a) a heavy chain variable region CDR1 comprising an amino acid sequence selected from the group consisting of SEQ ID NOs: 26 and 27, and conservative substitutions thereof;
(b) a heavy chain variable region CDR2 domain comprising an amino acid sequence selected from the group consisting of SEQ ID NOs: 30 and 31, and conservative substitutions thereof;
(c) a heavy chain variable region CDR3 domain comprising an amino acid sequence selected from the group consisting of SEQ ID NOs: 34 and 35, and conservative substitutions thereof;
(d) a light chain variable region CDR1 domain comprising an amino acid sequence selected from the group consisting of SEQ ID NOs: 38 and 39, and conservative substitutions thereof;
(e) a light chain variable region CDR2 domain comprising an amino acid sequence selected from the group consisting of SEQ ID NOs: 42 and 43, and conservative substitutions thereof; and
(f) a light chain variable region CDR3 domain comprising an amino acid sequence selected from the group consisting of SEQ ID NOs: 46 and 47, and conservative substitutions thereof.

48. The kit of any one of claims 32-46, wherein one or more of the capture antibody, first capture antibody and second capture antibody comprises:
(a) a heavy chain variable region comprising an amino acid sequence selected from the group consisting of SEQ ID NOs: 54, 55, 74 and 75, and conservative substitutions thereof; and
(b) a light chain variable region comprising an amino acid sequence selected from the group consisting of SEQ ID NOs: 50, 51, 70 and 71, and conservative substitutions thereof.

49. The kit of any one of claims 32-46, wherein one or more of the capture antibody, first capture antibody and second capture antibody comprises:
(a) a heavy chain comprising an amino acid sequence selected from the group consisting of SEQ ID NOs: 22, 23, 66 and 67, and conservative substitutions thereof; and
(b) a light chain comprising an amino acid sequence selected from the group consisting of SEQ ID NOs: 18, 19, 62 and 63, and conservative substitutions thereof.

50. The kit of any one of claims 32 and 35-46, wherein one or more of the detector antibody and first detector antibody comprises:
(a) a heavy chain variable region CDR1 comprising an amino acid sequence selected from the group consisting of SEQ ID NOs: 28 and 29, and conservative substitutions thereof;
(b) a heavy chain variable region CDR2 domain comprising an amino acid sequence selected from the group consisting of SEQ ID NOs: 32 and 33, and conservative substitutions thereof;
(c) a heavy chain variable region CDR3 domain comprising an amino acid sequence selected from the group consisting of SEQ ID NOs: 36 and 37, and conservative substitutions thereof;
(d) a light chain variable region CDR1 domain comprising an amino acid sequence selected from the group consisting of SEQ ID NOs: 40 and 41, and conservative substitutions thereof;
(e) a light chain variable region CDR2 domain comprising an amino acid sequence selected from the group consisting of SEQ ID NOs: 44 and 45, and conservative substitutions thereof; and
(f) a light chain variable region CDR3 domain comprising an amino acid sequence selected from the group consisting of SEQ ID NOs: 48 and 49, and conservative substitutions thereof.

51. The kit of any one of claims 32 and 35-46, wherein one or more of the detector antibody and first detector antibody comprises:
(a) a heavy chain variable region comprising an amino acid sequence selected from the group consisting of SEQ ID NOs: 56, 57, 72 and 73, and conservative substitutions thereof; and
(b) a light chain variable region comprising an amino acid sequence selected from the group consisting of SEQ ID NOs: 52, 53, 68 and 69, and conservative substitutions thereof.

52. The kit of any one of claims 32 and 35-46, wherein one or more of the detector antibody and first detector antibody comprises:
(a) a heavy chain comprising an amino acid sequence selected from the group consisting of SEQ ID NOs: 24, 25, 64 and 65, and conservative substitutions thereof; and
(b) a light chain comprising an amino acid sequence selected from the group consisting of SEQ ID NOs: 20, 21, 60 and 61, and conservative substitutions thereof.

53. The kit of claim 47, wherein one or more of the capture antibody, first capture antibody and second capture antibody comprises:
(a) a heavy chain variable region CDR1 comprising the amino acid sequence of SEQ ID NO: 26, and conservative substitutions thereof;
(b) a heavy chain variable region CDR2 domain comprising the amino acid sequence of SEQ ID NO: 30, and conservative substitutions thereof;
(c) a heavy chain variable region CDR3 domain comprising the amino acid sequence of SEQ ID NO: 34, and conservative substitutions thereof;
(d) a light chain variable region CDR1 domain comprising the amino acid sequence of SEQ ID NO: 38, and conservative substitutions thereof;
(e) a light chain variable region CDR2 domain comprising the amino acid sequence of SEQ ID NO: 42, and conservative substitutions thereof; and
(f) a light chain variable region CDR3 domain comprising the amino acid sequence of SEQ ID NO: 46, and conservative substitutions thereof.

54. The kit of claim 53, wherein one or more of the capture antibody, first capture antibody and second capture antibody comprises:
(a) a heavy chain variable region comprising the amino acid sequence of SEQ ID NO: 54, and conservative substitutions thereof; and
(b) a light chain variable region comprising the amino acid sequence of SEQ ID NO: 50, and conservative substitutions thereof.

55. The kit of claim 54, wherein one or more of the capture antibody, first capture antibody and second capture antibody comprises:
(a) a heavy chain comprising the amino acid sequence of SEQ ID NO: 22, and conservative substitutions thereof; and
(b) a light chain comprising the amino acid sequence of SEQ ID NO: 18, and conservative substitutions thereof.

56. The kit of claim 50, wherein one or more of the detector antibody and first detector antibody comprises:
(a) a heavy chain variable region CDR1 comprising the amino acid sequence of SEQ ID NO: 29, and conservative substitutions thereof;
(b) a heavy chain variable region CDR2 domain comprising the amino acid sequence of SEQ ID NO: 33, and conservative substitutions thereof;
(c) a heavy chain variable region CDR3 domain comprising the amino acid sequence of SEQ ID NO: 37, and conservative substitutions thereof;
(d) a light chain variable region CDR1 domain comprising the amino acid sequence of SEQ ID NO: 41, and conservative substitutions thereof;
(e) a light chain variable region CDR2 domain comprising the amino acid sequence of SEQ ID NO: 45, and conservative substitutions thereof; and
(f) a light chain variable region CDR3 domain comprising the amino acid sequence of SEQ ID NO: 49, and conservative substitutions thereof.

57. The kit of claim 56, wherein one or more of the detector antibody and first detector antibody comprises:
(a) a heavy chain variable region comprising the amino acid sequence of SEQ ID NO: 57, and conservative substitutions thereof; and
(b) a light chain variable region comprising the amino acid sequence of SEQ ID NO: 53, and conservative substitutions thereof.

58. The kit of claim 57, wherein one or more of the detector antibody and first detector antibody comprises:
(a) a heavy chain comprising the amino acid sequence of SEQ ID NO: 25, and conservative substitutions thereof; and
(b) a light chain comprising the amino acid sequence of SEQ ID NO: 21, and conservative substitutions thereof.

59. The kit of any one of claims 32-46, wherein one or more of the capture antibody, first capture antibody and second capture antibody competitively binds with an antibody comprising:
(a) a heavy chain variable region CDR1 comprising an amino acid sequence selected from the group consisting of SEQ ID NOs: 26 and 27, and conservative substitutions thereof;
(b) a heavy chain variable region CDR2 domain comprising an amino acid sequence selected from the group consisting of SEQ ID NOs: 30 and 31, and conservative substitutions thereof;
(c) a heavy chain variable region CDR3 domain comprising an amino acid sequence selected from the group consisting of SEQ ID NOs: 34 and 35, and conservative substitutions thereof;
(d) a light chain variable region CDR1 domain comprising an amino acid sequence selected from the group consisting of SEQ ID NOs: 38 and 39, and conservative substitutions thereof;
(e) a light chain variable region CDR2 domain comprising an amino acid sequence selected from the group consisting of SEQ ID NOs: 42 and 43, and conservative substitutions thereof; and
(f) a light chain variable region CDR3 domain comprising an amino acid sequence selected from the group consisting of SEQ ID NOs: 46 and 47, and conservative substitutions thereof.

60. The kit of any one of claims 32 and 35-46, wherein one or more of the detector antibody and first detector antibody competitively binds with an antibody comprising:
(a) a heavy chain variable region CDR1 comprising an amino acid sequence selected from the group consisting of SEQ ID NOs: 28 and 29, and conservative substitutions thereof;
(b) a heavy chain variable region CDR2 domain comprising an amino acid sequence selected from the group consisting of SEQ ID NOs: 32 and 33, and conservative substitutions thereof;
(c) a heavy chain variable region CDR3 domain comprising an amino acid sequence selected from the group consisting of SEQ ID NOs: 36 and 37, and conservative substitutions thereof;
(d) a light chain variable region CDR1 domain comprising an amino acid sequence selected from the group consisting of SEQ ID NOs: 40 and 41, and conservative substitutions thereof;
(e) a light chain variable region CDR2 domain comprising an amino acid sequence selected from the group consisting of SEQ ID NOs: 44 and 45, and conservative substitutions thereof; and
(f) a light chain variable region CDR3 domain comprising an amino acid sequence selected from the group consisting of SEQ ID NOs: 48 and 49, and conservative substitutions thereof.

61. The kit of any one of claims 32-60, wherein the sample is a blood sample.

62. The kit of any one of claims 32-60, wherein the sample is a plasma sample.

63. The kit of any one of claims 32-62, wherein the kit detects the amount of total or active FGF21 protein in the sample at an in-well sensitivity from about 0.2 pg/ml to about 0.5 pg/ml.

64. An isolated anti-FGF21 antibody, or an antigen-binding portion thereof, comprising:
(a) a heavy chain variable region CDR1 comprising an amino acid sequence selected from the group consisting of SEQ ID NOs: 26-29, and conservative substitutions thereof;
(b) a heavy chain variable region CDR2 domain comprising an amino acid sequence selected from the group consisting of SEQ ID NOs: 30-33, and conservative substitutions thereof;
(c) a heavy chain variable region CDR3 domain comprising an amino acid sequence selected from the group consisting of SEQ ID NOs: 34-37, and conservative substitutions thereof;
(d) a light chain variable region CDR1 domain comprising an amino acid sequence selected from the group consisting of SEQ ID NOs: 38-41, and conservative substitutions thereof;
(e) a light chain variable region CDR2 domain comprising an amino acid sequence selected from the group consisting of SEQ ID NOs: 42-45, and conservative substitutions thereof; and
(f) a light chain variable region CDR3 domain comprising an amino acid sequence selected from the group consisting of SEQ ID NOs: 46-49, and conservative substitutions thereof.

65. The isolated antibody of claim 64, wherein the antibody, or antigen-binding portion thereof, comprises:
(a) a heavy chain variable region CDR1 comprising the amino acid sequence of SEQ ID NO: 26, and conservative substitutions thereof;
(b) a heavy chain variable region CDR2 domain comprising the amino acid sequence of SEQ ID NO: 30, and conservative substitutions thereof;
(c) a heavy chain variable region CDR3 domain comprising the amino acid sequence of SEQ ID NO: 34, and conservative substitutions thereof;
(d) a light chain variable region CDR1 domain comprising the amino acid sequence of SEQ ID NO: 38, and conservative substitutions thereof;
(e) a light chain variable region CDR2 domain comprising the amino acid sequence of SEQ ID NO: 42, and conservative substitutions thereof; and
(f) a light chain variable region CDR3 domain comprising the amino acid sequence of SEQ ID NO: 46, and conservative substitutions thereof.

66. The isolated antibody of claim 64, wherein the antibody, or antigen-binding portion thereof, comprises:
(a) a heavy chain variable region CDR1 comprising the amino acid sequence of SEQ ID NO: 27, and conservative substitutions thereof;
(b) a heavy chain variable region CDR2 domain comprising the amino acid sequence of SEQ ID NO: 31, and conservative substitutions thereof;
(c) a heavy chain variable region CDR3 domain comprising the amino acid sequence of SEQ ID NO: 35, and conservative substitutions thereof;
(d) a light chain variable region CDR1 domain comprising the amino acid sequence of SEQ ID NO: 39, and conservative substitutions thereof;
(e) a light chain variable region CDR2 domain comprising the amino acid sequence of SEQ ID NO: 43, and conservative substitutions thereof; and
(f) a light chain variable region CDR3 domain comprising the amino acid sequence of SEQ ID NO: 47, and conservative substitutions thereof.

67. The isolated antibody of claim 64, wherein the antibody, or antigen-binding portion thereof, comprises:
(a) a heavy chain variable region CDR1 comprising the amino acid sequence of SEQ ID NO: 28, and conservative substitutions thereof;
(b) a heavy chain variable region CDR2 domain comprising the amino acid sequence of SEQ ID NO: 32, and conservative substitutions thereof;
(c) a heavy chain variable region CDR3 domain comprising the amino acid sequence of SEQ ID NO: 36, and conservative substitutions thereof;
(d) a light chain variable region CDR1 domain comprising the amino acid sequence of SEQ ID NO: 40, and conservative substitutions thereof;
(e) a light chain variable region CDR2 domain comprising the amino acid sequence of SEQ ID NO: 44, and conservative substitutions thereof; and
(f) a light chain variable region CDR3 domain comprising the amino acid sequence of SEQ ID NO: 48, and conservative substitutions thereof.

68. The isolated antibody of claim 64, wherein the antibody, or antigen-binding portion thereof, comprises:
(a) a heavy chain variable region CDR1 comprising the amino acid sequence of SEQ ID NO: 29, and conservative substitutions thereof;
(b) a heavy chain variable region CDR2 domain comprising the amino acid sequence of SEQ ID NO: 33, and conservative substitutions thereof;
(c) a heavy chain variable region CDR3 domain comprising the amino acid sequence of SEQ ID NO: 37, and conservative substitutions thereof;
(d) a light chain variable region CDR1 domain comprising the amino acid sequence of SEQ ID NO: 41, and conservative substitutions thereof;
(e) a light chain variable region CDR2 domain comprising the amino acid sequence of SEQ ID NO: 45, and conservative substitutions thereof; and
(f) a light chain variable region CDR3 domain comprising the amino acid sequence of SEQ ID NO: 49, and conservative substitutions thereof.

69. The isolated antibody of claim 65, wherein the antibody, or antigen-binding portion thereof, comprises:
(a) a heavy chain variable region comprising the amino acid sequence of SEQ ID NO: 54, and conservative substitutions thereof; and
(b) a light chain variable region comprising the amino acid sequence of SEQ ID NO: 50, and conservative substitutions thereof.

70. The isolated antibody of claim 66, wherein the antibody, or antigen-binding portion thereof, comprises:
(a) a heavy chain variable region comprising the amino acid sequence of SEQ ID NO: 55, and conservative substitutions thereof; and
(b) a light chain variable region comprising the amino acid sequence of SEQ ID NO: 51, and conservative substitutions thereof.

71. The isolated antibody of claim 67, wherein the antibody, or antigen-binding portion thereof, comprises:
(a) a heavy chain variable region comprising the amino acid sequence of SEQ ID NO: 56, and conservative substitutions thereof; and
(b) a light chain variable region comprising the amino acid sequence of SEQ ID NO: 52, and conservative substitutions thereof.

72. The isolated antibody of claim 68, wherein the antibody, or antigen-binding portion thereof, comprises:
(a) a heavy chain variable region comprising the amino acid sequence of SEQ ID NO: 57, and conservative substitutions thereof; and
(b) a light chain variable region comprising the amino acid sequence of SEQ ID NO: 53, and conservative substitutions thereof.

73. The isolated antibody of claim 65, wherein the antibody, or antigen-binding portion thereof, comprises:
(a) a heavy chain variable region comprising the amino acid sequence of SEQ ID NO: 75, and conservative substitutions thereof; and
(b) a light chain variable region comprising the amino acid sequence of SEQ ID NO: 71, and conservative substitutions thereof.

74. The isolated antibody of claim 66, wherein the antibody, or antigen-binding portion thereof, comprises:
(a) a heavy chain variable region comprising the amino acid sequence of SEQ ID NO: 74, and conservative substitutions thereof; and
(b) a light chain variable region comprising the amino acid sequence of SEQ ID NO: 70, and conservative substitutions thereof.

75. The isolated antibody of claim 67, wherein the antibody, or antigen-binding portion thereof, comprises:
(a) a heavy chain variable region comprising the amino acid sequence of SEQ ID NO: 73, and conservative substitutions thereof; and
(b) a light chain variable region comprising the amino acid sequence of SEQ ID NO: 69, and conservative substitutions thereof.

76. The isolated antibody of claim 68, wherein the antibody, or antigen-binding portion thereof, comprises:
(a) a heavy chain variable region comprising the amino acid sequence of SEQ ID NO: 72, and conservative substitutions thereof; and
(b) a light chain variable region comprising the amino acid sequence of SEQ ID NO: 68, and conservative substitutions thereof.

77. The isolated antibody of claim 69, wherein the antibody, or antigen-binding portion thereof, comprises:
(a) a heavy chain comprising the amino acid sequence of SEQ ID NO: 22, and conservative substitutions thereof; and
(b) a light chain comprising the amino acid sequence of SEQ ID NO: 18, and conservative substitutions thereof.

78. The isolated antibody of claim 70, wherein the antibody, or antigen-binding portion thereof, comprises:
(a) a heavy chain comprising the amino acid sequence of SEQ ID NO: 23, and conservative substitutions thereof; and
(b) a light chain comprising the amino acid sequence of SEQ ID NO: 19, and conservative substitutions thereof.

79. The isolated antibody of claim 71, wherein the antibody, or antigen-binding portion thereof, comprises:
(a) a heavy chain comprising the amino acid sequence of SEQ ID NO: 24, and conservative substitutions thereof; and
(b) a light chain comprising the amino acid sequence of SEQ ID NO: 20, and conservative substitutions thereof.

80. The isolated antibody of claim 72, wherein the antibody, or antigen-binding portion thereof, comprises:
(a) a heavy chain comprising the amino acid sequence of SEQ ID NO: 25, and conservative substitutions thereof; and
(b) a light chain comprising the amino acid sequence of SEQ ID NO: 21, and conservative substitutions thereof.

81. The isolated antibody of claim 73, wherein the antibody, or antigen-binding portion thereof, comprises:
(a) a heavy chain comprising the amino acid sequence of SEQ ID NO: 67, and conservative substitutions thereof; and
(b) a light chain comprising the amino acid sequence of SEQ ID NO: 63, and conservative substitutions thereof.

82. The isolated antibody of claim 74, wherein the antibody, or antigen-binding portion thereof, comprises:
(a) a heavy chain comprising the amino acid sequence of SEQ ID NO: 66, and conservative substitutions thereof; and
(b) a light chain comprising the amino acid sequence of SEQ ID NO: 62, and conservative substitutions thereof.

83. The isolated antibody of claim 75, wherein the antibody, or antigen-binding portion thereof, comprises:
(a) a heavy chain comprising the amino acid sequence of SEQ ID NO: 65, and conservative substitutions thereof; and
(b) a light chain comprising the amino acid sequence of SEQ ID NO: 61, and conservative substitutions thereof.

84. The isolated antibody of claim 76, wherein the antibody, or antigen-binding portion thereof, comprises:
(a) a heavy chain comprising the amino acid sequence of SEQ ID NO: 64, and conservative substitutions thereof; and
(b) a light chain comprising the amino acid sequence of SEQ ID NO: 60, and conservative substitutions thereof.

85. An isolated nucleic acid encoding the antibody, or antigen-binding portion thereof, of any one of claims 64-84.

86. A host cell comprising the nucleic acid of claim 85.

87. A method of producing an antibody comprising culturing the host cell of claim 86 so that the antibody is produced.

88. The method of claim 87, further comprising recovering the antibody from the host cell.

89. A composition comprising one or more antibodies, or antigen-binding portions thereof, of any one of claims 64-84.


SEQUENCE LISTING (77 sequences)

SEQ ID NO: 1 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 3 - protein, 192 aa, Homo sapiens
HPIPDSSPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 4 - protein, 189 aa, Artificial Sequence
  source: /note=“Description of Artificial Sequence Synthetic polypeptide”
HPIPDSSPLLQFGGQVRQRYLYTDDAQQTEAHLEIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 5 - protein, 189 aa, Artificial Sequence
  source: /note=“Description of Artificial Sequence Synthetic polypeptide”
HPIPDSSPLLQFGGQVRQRYLYTDDAQQTEAHLEIREDGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 6 - protein, 189 aa, Artificial Sequence
  source: /note=“Description of Artificial Sequence Synthetic polypeptide”
HPIPDSSPLLQFGGQVRQRYLYTDDAQQTEAHLEIREDGTVGGAADQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 7 - protein, 189 aa, Artificial Sequence
  source: /note=“Description of Artificial Sequence Synthetic polypeptide”
HPIPDSSPLLQFGGQVRQRYLYTDDAQQTEAHLEIREDGTVGGAADQSPESLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 8 - protein, 189 aa, Artificial Sequence
  source: /note=“Description of Artificial Sequence Synthetic polypeptide”
HPIPDSSPLLQFGGQVRQRYLYTDDAQQTEAHLEIREDGTVGGAADQSPESLLQLKALKPGVIQILGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 9 - protein, 189 aa, Artificial Sequence
  source: /note=“Description of Artificial Sequence Synthetic polypeptide”
HPIPDSSPLLQFGGQVRQRYLYTDDAQQTEAHLEIREDGTVGGAADQSPESLLQLKALKPGVIQILGVKTSRFLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 10 - protein, 187 aa, Artificial Sequence
  source: /note=“Description of Artificial Sequence Synthetic polypeptide”
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPMLPMVPEEPEDLRGHLESDMFSSPLETDSMDPFGLVTGLEAVRSPSFEK

SEQ ID NO: 11 - protein, 183 aa, Artificial Sequence
  source: /note=“Description of Artificial Sequence Synthetic polypeptide”
RPLAFSDAGPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 12 - protein, 186 aa, Artificial Sequence
  source: /note=“Description of Artificial Sequence Synthetic polypeptide”
RPLAFSDAGPHVHYGWGDPIRLRHLYTSGPHGLSSCFLRIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 13 - protein, 186 aa, Artificial Sequence
  source: /note=“Description of Artificial Sequence Synthetic polypeptide”
RPLAFSDAGPHVHYGWGDPIRLRHLYTSGPHGLSSCFLRIRADGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 14 - protein, 186 aa, Artificial Sequence
  source: /note=“Description of Artificial Sequence Synthetic polypeptide”
RPLAFSDAGPHVHYGWGDPIRLRHLYTSGPHGLSSCFLRIRADGVVDCARGQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 15 - protein, 186 aa, Artificial Sequence
  source: /note=“Description of Artificial Sequence Synthetic polypeptide”
RPLAFSDAGPHVHYGWGDPIRLRHLYTSGPHGLSSCFLRIRADGVVDCARGQSAHSLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 16 - protein, 186 aa, Artificial Sequence
  source: /note=“Description of Artificial Sequence Synthetic polypeptide”
RPLAFSDAGPHVHYGWGDPIRLRHLYTSGPHGLSSCFLRIRADGVVDCARGQSAHSLLEIKAVALRTVAIKGVHSVRYLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 17 - protein, 188 aa, Artificial Sequence
  source: /note=“Description of Artificial Sequence Synthetic polypeptide”
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPLPGLPPALPEPPGILAPQPPDVGSSDPLSMVGPSQGRSPSYAS

SEQ ID NO: 18 - protein, 217 aa, Artificial Sequence
  source: /note=“Description of Artificial Sequence Synthetic polypeptide”
QIVLTQSPAIMSAPLGERVTMTCTASSSVSSSYLHWYQQKPGSSPKVWIYRTTNLASGVPTRFSGSGSGTSYSLTISSMEAEDAATYYCHQYHRSPPTWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 214 aa, Artificial Sequence
  source: /note=“Description of Artificial Sequence Synthetic polypeptide”
DIQMTQSPASLSASVGETVIITCRASENIYSYLAWYQQKQGKSPQLLVYNIRTLAEGVPSRFSGSGSGTQFSLKINSLQPEDFGSYYCQHHYDSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 213 aa, Artificial Sequence
  source: /note=“Description of Artificial Sequence Synthetic polypeptide”
QIVLTQSPALMSASPGERVTMTCSAGSSVSYMYWYQQKPRSSPKPWIYLTSNLASGVPARFSGSGSGTSYSLTISSMEAEDAATYYCQQWSSNPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 219 aa, Artificial Sequence
  source: /note=“Description of Artificial Sequence Synthetic polypeptide”
DVLMTQTPLSLPVSLGDQASISCRSSQIIVHNNGDTYLEWYLQKPGQSPKLLIYKISNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 449 aa, Artificial Sequence
  source: /note=“Description of Artificial Sequence Synthetic polypeptide”
EVKLVESGGDLVKPGGSLKLSCAASGFTFSSYGMSWVRQTPDKRLEWVATISTGGGYTYYPDSVKGRFTISRDNAKNTLYLQMSSLRSEDTAMYYCARHDLVDWYFDVWGTGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 23 - protein, 447 aa, Artificial Sequence
  source: /note=“Description of Artificial Sequence Synthetic polypeptide”
EVQLQQSGPELVKPGASVKIPCKASGYTFTDYYMGWVKQSHGKSLEWIGDINPNNGVTINNQNFKGKATLTVDKSSSTAYMELRSLASEDTAVYYCTRGYGGALDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - protein, 447 aa, Artificial Sequence
  source: /note=“Description of Artificial Sequence Synthetic polypeptide”
QVQLQQSGAELARPGASVKLSCKASGYTFTNYGISWVKQRTGQGLEWIGEIYPRSDNTYYNEKFKGKATLTADKSSSTAYMELRSLTSEDSAVYFCTRSDYGFFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 25 - protein, 447 aa, Artificial Sequence
  source: /note=“Description of Artificial Sequence Synthetic polypeptide”
QVQLIQSGPGLVQPSQSLSITCTVSGFSLTGYAIHWVRQSPGKGLEWLGMIWKSGNTDYNAAFMSRLSITKDNSKSQVFFKMNSLQADDTAIYYCARNGYDYEFVYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - protein, 5 aa, Artificial Sequence
  source: /note=“Description of Artificial Sequence Synthetic peptide”
SYGMS

SEQ ID NO: 27 - protein, 5 aa, Artificial Sequence
  source: /note=“Description of Artificial Sequence Synthetic peptide”
DYYMG

SEQ ID NO: 28 - protein, 5 aa, Artificial Sequence
  source: /note=“Description of Artificial Sequence Synthetic peptide”
NYGIS

SEQ ID NO: 29 - protein, 5 aa, Artificial Sequence
  source: /note=“Description of Artificial Sequence Synthetic peptide”
GYAIH

SEQ ID NO: 30 - protein, 17 aa, Artificial Sequence
  source: /note=“Description of Artificial Sequence Synthetic peptide”
TISTGGGYTYYPDSVKG

SEQ ID NO: 31 - protein, 17 aa, Artificial Sequence
  source: /note=“Description of Artificial Sequence Synthetic peptide”
DINPNNGVTINNQNFKG

SEQ ID NO: 32 - protein, 17 aa, Artificial Sequence
  source: /note=“Description of Artificial Sequence Synthetic peptide”
EIYPRSDNTYYNEKFKG

SEQ ID NO: 33 - protein, 16 aa, Artificial Sequence
  source: /note=“Description of Artificial Sequence Synthetic peptide”
MIWKSGNTDYNAAFMS

SEQ ID NO: 34 - protein, 10 aa, Artificial Sequence
  source: /note=“Description of Artificial Sequence Synthetic peptide”
HDLVDWYFDV

SEQ ID NO: 35 - protein, 8 aa, Artificial Sequence
  source: /note=“Description of Artificial Sequence Synthetic peptide”
GYGGALDY

SEQ ID NO: 36 - protein, 8 aa, Artificial Sequence
  source: /note=“Description of Artificial Sequence Synthetic peptide”
SDYGFFDY

SEQ ID NO: 37 - protein, 9 aa, Artificial Sequence
  source: /note=“Description of Artificial Sequence Synthetic peptide”
NGYDYEFVY

SEQ ID NO: 38 - protein, 12 aa, Artificial Sequence
  source: /note=“Description of Artificial Sequence Synthetic peptide”
TASSSVSSSYLH

SEQ ID NO: 39 - protein, 11 aa, Artificial Sequence
  source: /note=“Description of Artificial Sequence Synthetic peptide”
RASENIYSYLA

SEQ ID NO: 40 - protein, 10 aa, Artificial Sequence
  source: /note=“Description of Artificial Sequence Synthetic peptide”
SAGSSVSYMY

SEQ ID NO: 41 - protein, 16 aa, Artificial Sequence
  source: /note=“Description of Artificial Sequence Synthetic peptide”
RSSQIIVHNNGDTYLE

SEQ ID NO: 42 - protein, 7 aa, Artificial Sequence
  source: /note=“Description of Artificial Sequence Synthetic peptide”
RTTNLAS

SEQ ID NO: 43 - protein, 7 aa, Artificial Sequence
  source: /note=“Description of Artificial Sequence Synthetic peptide”
NIRTLAE

SEQ ID NO: 44 - protein, 7 aa, Artificial Sequence
  source: /note=“Description of Artificial Sequence Synthetic peptide”
LTSNLAS

SEQ ID NO: 45 - protein, 7 aa, Artificial Sequence
  source: /note=“Description of Artificial Sequence Synthetic peptide”
KISNRFS

SEQ ID NO: 46 - protein, 11 aa, Artificial Sequence
  source: /note=“Description of Artificial Sequence Synthetic peptide”
HQYHRSPPTWT

SEQ ID NO: 47 - protein, 9 aa, Artificial Sequence
  source: /note=“Description of Artificial Sequence Synthetic peptide”
QHHYDSPWT

SEQ ID NO: 48 - protein, 9 aa, Artificial Sequence
  source: /note=“Description of Artificial Sequence Synthetic peptide”
QQWSSNPRT

SEQ ID NO: 49 - protein, 9 aa, Artificial Sequence
  source: /note=“Description of Artificial Sequence Synthetic peptide”
FQGSHVPYT

SEQ ID NO: 50 - protein, 110 aa, Artificial Sequence
  source: /note=“Description of Artificial Sequence Synthetic polypeptide”
QIVLTQSPAIMSAPLGERVTMTCTASSSVSSSYLHWYQQKPGSSPKVWIYRTTNLASGVPTRFSGSGSGTSYSLTISSMEAEDAATYYCHQYHRSPPTWTFGGGTKLEIK

SEQ ID NO: 51 - protein, 107 aa, Artificial Sequence
  source: /note=“Description of Artificial Sequence Synthetic polypeptide”
DIQMTQSPASLSASVGETVIITCRASENIYSYLAWYQQKQGKSPQLLVYNIRTLAEGVPSRFSGSGSGTQFSLKINSLQPEDFGSYYCQHHYDSPWTFGGGTKLEIK

SEQ ID NO: 52 - protein, 106 aa, Artificial Sequence
  source: /note=“Description of Artificial Sequence Synthetic polypeptide”
QIVLTQSPALMSASPGERVTMTCSAGSSVSYMYWYQQKPRSSPKPWIYLTSNLASGVPARFSGSGSGTSYSLTISSMEAEDAATYYCQQWSSNPRTFGGGTKLEIK

SEQ ID NO: 53 - protein, 112 aa, Artificial Sequence
  source: /note=“Description of Artificial Sequence Synthetic polypeptide”
DVLMTQTPLSLPVSLGDQASISCRSSQIIVHNNGDTYLEWYLQKPGQSPKLLIYKISNRFSGVPDRFSGSGSGTDFTLKISRVEAEDLGVYYCFQGSHVPYTFGGGTKLEIK

SEQ ID NO: 54 - protein, 119 aa, Artificial Sequence
  source: /note=“Description of Artificial Sequence Synthetic polypeptide”
EVKLVESGGDLVKPGGSLKLSCAASGFTFSSYGMSWVRQTPDKRLEWVATISTGGGYTYYPDSVKGRFTISRDNAKNTLYLQMSSLRSEDTAMYYCARHDLVDWYFDVWGTGTTVTVSS

SEQ ID NO: 55 - protein, 117 aa, Artificial Sequence
  source: /note=“Description of Artificial Sequence Synthetic polypeptide”
EVQLQQSGPELVKPGASVKIPCKASGYTFTDYYMGWVKQSHGKSLEWIGDINPNNGVTINNQNFKGKATLTVDKSSSTAYMELRSLASEDTAVYYCTRGYGGALDYWGQGTSVTVSS

SEQ ID NO: 56 - protein, 117 aa, Artificial Sequence
  source: /note=“Description of Artificial Sequence Synthetic polypeptide”
QVQLQQSGAELARPGASVKLSCKASGYTFTNYGISWVKQRTGQGLEWIGEIYPRSDNTYYNEKFKGKATLTADKSSSTAYMELRSLTSEDSAVYFCTRSDYGFFDYWGQGTTLTVSS

SEQ ID NO: 57 - protein, 117 aa, Artificial Sequence
  source: /note=“Description of Artificial Sequence Synthetic polypeptide”
QVQLIQSGPGLVQPSQSLSITCTVSGFSLTGYAIHWVRQSPGKGLEWLGMIWKSGNTDYNAAFMSRLSITKDNSKSQVFFKMNSLQADDTAIYYCARNGYDYEFVYWGQGTLVTVSA

SEQ ID NO: 58 - protein, 171 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGP

SEQ ID NO: 59 - protein, 177 aa, Homo sapiens
DSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 60 - protein, 219 aa, Artificial Sequence
  source: /note=“Description of Artificial Sequence Synthetic polypeptide”
DVLMTQTPLSLPVSLGDQASISCRSSQIIVHNNGDTYLEWYLQKPGQSPKLLIYKISNRFSGVPDRFSGSGSGTDFTLKISRVEAEDLGVYYCFQGSHVPYTFGGGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 61 - protein, 213 aa, Artificial Sequence
  source: /note=“Description of Artificial Sequence Synthetic polypeptide”
QIVLTQSPALMSASPGERVTMTCSAGSSVSYMYWYQQKPRSSPKPWIYLTSNLASGVPARFSGSGSGTSYSLTISSMEAEDAATYYCQQWSSNPRTFGGGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 62 - protein, 214 aa, Artificial Sequence
  source: /note=“Description of Artificial Sequence Synthetic polypeptide”
DIQMTQSPASLSASVGETVIITCRASENIYSYLAWYQQKQGKSPQLLVYNIRTLAEGVPSRFSGSGSGTQFSLKINSLQPEDFGSYYCQHHYDSPWTFGGGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 63 - protein, 217 aa, Artificial Sequence
  source: /note=“Description of Artificial Sequence Synthetic polypeptide”
QIVLTQSPAIMSAPLGERVTMTCTASSSVSSSYLHWYQQKPGSSPKVWIYRTTNLASGVPTRFSGSGSGTSYSLTISSMEAEDAATYYCHQYHRSPPTWTFGGGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 64 - protein, 447 aa, Artificial Sequence
  source: /note=“Description of Artificial Sequence Synthetic polypeptide”
QVQLIQSGPGLVQPSQSLSITCTVSGFSLTGYAIHWVRQSPGKGLEWLGMIWKSGNTDYNAAFMSRLSITKDNSKSQVFFKMNSLQADDTAIYYCARNGYDYEFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 447 aa, Artificial Sequence
  source: /note=“Description of Artificial Sequence Synthetic polypeptide”
QVQLQQSGAELARPGASVKLSCKASGYTFTNYGISWVKQRTGQGLEWIGEIYPRSDNTYYNEKFKGKATLTADKSSSTAYMELRSLTSEDSAVYFCTRSDYGFFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 447 aa, Artificial Sequence
  source: /note=“Description of Artificial Sequence Synthetic polypeptide”
EVQLQQSGPELVKPGASVKIPCKASGYTFTDYYMGWVKQSHGKSLEWIGDINPNNGVTINNQNFKGKATLTVDKSSSTAYMELRSLASEDTAVYYCTRGYGGALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 449 aa, Artificial Sequence
  source: /note=“Description of Artificial Sequence Synthetic polypeptide”
EVKLVESGGDLVKPGGSLKLSCAASGFTFSSYGMSWVRQTPDKRLEWVATISTGGGYTYYPDSVKGRFTISRDNAKNTLYLQMSSLRSEDTAMYYCARHDLVDWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 112 aa, Artificial Sequence
  source: /note=“Description of Artificial Sequence Synthetic polypeptide”
DVLMTQTPLSLPVSLGDQASISCRSSQIIVHNNGDTYLEWYLQKPGQSPKLLIYKISNRFSGVPDRFSGSGSGTDFTLKISRVEAEDLGVYYCFQGSHVPYTFGGGTKVEIK

SEQ ID NO: 69 - protein, 98 aa, Artificial Sequence
  source: /note=“Description of Artificial Sequence Synthetic polypeptide”
ALMSASPGERVTMTCSAGSSVSYMYWYQQKPRSSPKPWIYLTSNLASGVPARFSGSGSGTSYSLTISSMEAEDAATYYCQQWSSNPRTFGGGTKVEIK

SEQ ID NO: 70 - protein, 107 aa, Artificial Sequence
  source: /note=“Description of Artificial Sequence Synthetic polypeptide”
DIQMTQSPASLSASVGETVIITCRASENIYSYLAWYQQKQGKSPQLLVYNIRTLAEGVPSRFSGSGSGTQFSLKINSLQPEDFGSYYCQHHYDSPWTFGGGTKVEIK

SEQ ID NO: 71 - protein, 110 aa, Artificial Sequence
  source: /note=“Description of Artificial Sequence Synthetic polypeptide”
QIVLTQSPAIMSAPLGERVTMTCTASSSVSSSYLHWYQQKPGSSPKVWIYRTTNLASGVPTRFSGSGSGTSYSLTISSMEAEDAATYYCHQYHRSPPTWTFGGGTKVEIK

SEQ ID NO: 72 - protein, 117 aa, Artificial Sequence
  source: /note=“Description of Artificial Sequence Synthetic polypeptide”
QVQLIQSGPGLVQPSQSLSITCTVSGFSLTGYAIHWVRQSPGKGLEWLGMIWKSGNTDYNAAFMSRLSITKDNSKSQVFFKMNSLQADDTAIYYCARNGYDYEFVYWGQGTLVTVSS

SEQ ID NO: 73 - protein, 117 aa, Artificial Sequence; identical to SEQ ID NO: 56
  source: /note=“Description of Artificial Sequence Synthetic polypeptide”

SEQ ID NO: 74 - protein, 117 aa, Artificial Sequence; identical to SEQ ID NO: 55
  source: /note=“Description of Artificial Sequence Synthetic polypeptide”

SEQ ID NO: 75 - protein, 119 aa, Artificial Sequence; identical to SEQ ID NO: 54
  source: /note=“Description of Artificial Sequence Synthetic polypeptide”

SEQ ID NO: 76 - protein, 4 aa, Homo sapiens
HPIP

SEQ ID NO: 77 - protein, 10 aa, Homo sapiens
SQGRSPSYAS